(19) United States
(12) Patent Application Publication
(10) Pub. No.: US 2016/0000737 A1
(43) Pub. Date: Jan. 7, 2016

(54) TREATMENT OF PETS WITH SIRTUIN ACTIVATORS

(71) Applicant: NUSIRT SCIENCES, INC., Nashville, TN (US)

(72) Inventors: Michael ZEMEL, Knoxville, TN (US); Antie BRUCKBAUER, Xnoxville, TN (US)

(21) Appl. No.: 14/770,418

(22) PCT Filed: Feb. 14, 2014
(86) PCT No.: PCT/US14/16592

§ 371 Date: Aug. 25, 2015

Related U.S. Application Data
(60) Provisional application No. 61/800,266, filed on Mar. 15, 2013.

(51) Int. Cl. A61K 31/198; A61K 31/355; A61K 31/375; A61K 31/205; A61K 31/385; A61K 33/32; A61K 36/9068; A61K 36/28; A61K 31/726; A61K 31/7008; A61K 36/61; A61K 35/741; A61K 31/05; A23K 1/16; A23K 1/14; A23K 1/00; A23K 1/18; A61K 31/202

(52) U.S. Cl. CPC A61K 31/198; A61K 31/202; A61K 31/355; A61K 31/375; A61K 31/205; A61K 31/385; A61K 33/32; A61K 36/9068; A61K 36/28; A61K 31/726; A61K 31/7008; A61K 36/61; A61K 35/741; A61K 31/05; A23K 1/1634; A23K 1/1603; A23K 1/164; A23K 1/1646; A23K 1/14; A23K 1/009; A23K 1/1846; A23K 1/004; A61K 2035/115

(57) ABSTRACT

The present invention provides for systems, compositions, methods and kits for regulating energy metabolism in pets. The systems, compositions, and kits can comprise, and methods can make use of, pet foods, pet treats, pet supplements, and pet drinks. In one aspect, the invention provides for pet food, treat, supplement, and drink compositions that comprise a combination of (a) leucine, and (b) a sirtuin activator, or any precursors or metabolites of (a) or (b). These combinations may be effective for reducing weight or adipose volume in the pet. In another aspect, the invention provides for methods of regulating energy metabolism by the administration of one or more compositions comprising leucine, a leucine metabolite, and/or a sirtuin activator. The invention also provides for kits comprising compositions of leucine, a leucine metabolite, and/or a sirtuin activator packaged in an oral dose form with usage instructions.

### CROSS-REFERENCE TO RELATED APPLICATIONS

This application claims benefit from U.S. Patent Application No. 61/800,266 (Attorney Docket No. 42736-708.101), filed Mar. 15, 2013, the full disclosure of which is incorporated herein by reference.

### BACKGROUND OF THE INVENTION

Studies have shown that a significant portion of companion animal (e.g., dog and cat) populations are overweight or obese. Obesity in domesticated dogs and cats has been linked to the development of numerous diseases including renal failure, diabetes, and arthritis. Overweight dogs have an increased risk of developing transitional cell carcinoma of the bladder. Further, it is well established that obesity is a predisposing factor to idiopathic hepatic lipidosis in cats.

Generally, obesity is considered present when body weight of the companion pet is 15% or more greater than optimum, which is the point at which health problems begin increasing with increasing weight. Generally speaking, the incidence of obesity in domestic animals increases with age. Similar to humans, as a dog ages, body fat increases, and lean body mass decreases. However, obesity commonly goes unnoticed by the animal's owner and, thus, poses a life-threatening problem to domesticated animals.

Main meal pet foods are usually sold as complete and balanced foods that fulfill nutritional requirements for the pet. Average required daily caloric intakes for animals are based on their body weight. Average required daily nutrient intakes are generally based on caloric intake. The serving sizes of food generally vary according to an animal's weight or may be targeted for specific breeds, specific sizes of animals or ages of the animals. Complete and balanced serving sizes of pet foods mean that when fed to an animal's caloric requirements for the animal's weight, the animal receives all of its required daily nutritional and caloric requirements.

Current weight loss diets for companion animals (e.g., dogs) rely on severe calorie restrictions and caloric dilution for effectiveness. However, the metabolism of many animals cannot tolerate calorie restriction diets, and they can end up causing more harm than good. For example, caloric restriction can trigger metabolic changes in liver fat storage. As the animal stores more fat in its liver, it reduces the capacity for normal healthy cells to grow which can lead to hepatitis.

Additionally, many companion animal weight loss products require a prescription by a veterinarian. This can be disadvantageous because it makes pet weight management a costly and time-consuming effort for the owner. Accordingly, there remains a great need for effective compositions, methods, and kits for the safe regulation of energy metabolism and/or associated metabolic disorders in pets.

### SUMMARY OF THE INVENTION

The present invention generally relates to the field of regulation of energy metabolism in pets. In some embodiments, the present invention provides for compositions, methods, and kits for regulating energy metabolism using leucine and/or leucine metabolites in combination with a sirtuin activator.

The present invention addresses the need for improved compositions and supplements for regulating energy metabolism in pets. The regulation of energy metabolism can allow for decreases in weight or adipose tissue, increases in fat oxidation or insulin sensitivity, and/or the decrease of inflammation or oxidative stress in pets. These effects can be by way of an increase in or regulation of a pet's energy metabolism, including cellular metabolism and mitochondrial biogenesis.

The subject compositions can be administered to a pet orally or through other routes such as intravenous administration. Compositions for oral administration can include pet food, pet treats, and pet supplements. Alternatively, the subject compositions can be in a form that can be mixed with or supplemented into a pet food by a pet owner.

In some embodiments, a pet food composition comprises: about 0.05 to 5 wt % of leucine and/or about 0.005 to 1 wt % of one or more leucine metabolites; and about 0.0005 to 0.05 wt % of a sirtuin activator. In some embodiments, a pet food, treat or supplement composition comprises: at least about 50 mg of leucine and/or at least about 5 mg of one or more leucine metabolites; and at least about 1 mg of a sirtuin activator. In some embodiments, a pet food composition comprises about 0.05 to 5 wt % of leucine and/or about 0.005 to 1 wt % of one or more leucine metabolites; about 0.0005 to 0.05 wt % of a sirtuin activator; and an additional component selected from the group consisting of omega-3 fatty acid, eicosapentanoic acid, choline, manganese, methionine, cysteine, L-carnitine, lysine, alpha lipoic acid, dimethylaminoethanol, pyruvic acid, actyl L-carnitine, L-carnitine, conjugated linoleic acid, diacylglyceride, chondroitin, glucosamine, ginger (or extract thereof), chicory pulp, and myrtle. In some embodiments, a pet food, treat or supplement composition comprises: at least about 50 mg of leucine and/or at least about 5 mg of one or more leucine metabolites; at least about 1 mg of a sirtuin activator; and an additional component selected from the group consisting of omega-3 fatty acid, eicosapentanoic acid, choline, manganese, methionine, cysteine, L-carnitine, lysine, alpha lipoic acid, dimethylaminoethanol, pyruvic acid, actyl L-carnitine, L-carnitine, conjugated linoleic acid, diacylglyceride, chondroitin, glucosamine, ginger (or extract thereof), chicory pump, and myrtle.

In some embodiments, said omega-3 fatty acid comprises at least about 0.05%, at least about 3.5%, or between about 0.05 to 3.5% of the weight of the pet food composition. In some embodiments, said eicosapentanoic acid comprises at least about 0.4 wt % of the pet food composition. In some embodiments, said vitamin E comprises at least about 100 ppm of the pet food composition. In some embodiments, said vitamin C comprises at least about 50 ppm of the pet food composition. In some embodiments, said L-carnitine comprises at least about 50 ppm of the pet food composition. In some embodiments, said alpha-lipoic acid comprises at least about 25 ppm of the pet food composition. In some embodiments, said choline comprises at least about 1000 ppm of the pet food composition. In some embodiments, said manganese comprises at least about 50 ppm, or from about 50 ppm to about 150 ppm, or from about 100 ppm to about 150 ppm, or from about 100 ppm to about 110 ppm of the pet food composition. In some embodiments, said methionine comprises at least about at least about 0.4 to 1.5% methionine by weight of the pet food composition. In some embodiments, said lysine comprises at least about 0.4%, between about 0.4 to 2%, between about 0.9 to 2%, or between about 0.9 to 1.2% by weight of the pet food composition. In some embodiments, said amount of ginger or extract thereof is about 0.005 to 12% by weight of the pet food composition. In some embodiments, said chicory pulp comprises from 0.5 to 20% dry weight of the pet food composition. In some embodiments, said chondroitin comprises at least about 0.5 wt % of the pet food composition. In some embodiments, said glucosamine comprises at least about 0.3 wt % of the pet food composition. In some embodiments, said myrtle comprises at least about 1% or between about 1 to 10% by weight of the pet food composition.

In some embodiments, said omega-3 fatty acid is present in an amount of at least about 250 mg. In some embodiments, said eicosapentanoic acid is present in an amount of at least about 300 mg. In some embodiments, said vitamin E is present in an amount of at least about 200 mg. In some embodiments, said vitamin C is present in an amount of at least about 250 mg. In some embodiments, said L-carnitine is present in an amount of at least about 250 mg. In some embodiments, said alpha-lipoic acid is present in an amount of at least about 180 mg. In some embodiments, said choline is present in an amount of at least about 1000 mg. In some embodiments, said manganese is present in an amount of at least about 50 mg, or from about 50 mg to about 150 mg, or from about 100 mg to about 150 mg, or from about 100 mg to about 110 mg. In some embodiments, said methionine is present in an amount of at least about 7.5 mg. In some embodiments, said lysine is present in an amount of at least about 400 mg. In some embodiments, said amount of ginger or extract thereof is present at an amount of at least about 500 mg. In some embodiments, said chondroitin is present in an amount of at least about 500 mg. In some embodiments, said glucosamine is present in an amount of at least about 300 mg. In some embodiments, said myrtle is present in an amount of at least about 750 mg.

In some embodiments, a pet food composition comprises: about 0.05 to 5 wt. % of leucine and/or about 0.005 to 1 wt % of one or more leucine metabolites on a dry matter basis; about 0.0005 to 0.05 wt. % of a sirtuin activator on a dry matter basis; about 26 to 35 wt. % of crude protein on a dry matter basis; about 7.5 to 8.5 wt. % of crude fat on a dry matter basis; about 20 to 30 wt. % of total dietary fiber on a dry matter basis; and about 10 to 20 wt. % of crude fiber on a dry matter basis.

In some embodiments, a pet food composition comprises: N pieces wherein N pieces provide the complete and balanced daily nutritional requirements of an animal, and N equals 1 to less than 15 pieces, wherein each piece has a caloric content between 50 to 2500 kcal, and wherein N pieces comprise at least about 100 mg of leucine and/or 10 mg of one or more leucine metabolites and at least about 1 mg of a sirtuin activator.

In some embodiments, a diet for promoting comprehensive weight management in companion animals comprises: a first stage pet food composition for promoting weight loss and a second stage pet food composition for maintaining the weight loss, (a) said first stage pet food composition comprising, on a dry matter basis, about 35 to 70% by weight of a protein, about 4 to 10% by weight of a fat, about 2 to 25% by weight of a fiber, about 10 to 35% by weight of a carbohydrate, about 0.05 to 5% by weight of leucine and/or about 0.005 to 1% by weight of one or more leucine metabolites, about 0.0005 to 0.05% by weight of a sirtuin activator, and about 0.1 to 2% by weight of a functional ingredient, wherein said functional ingredient is selected from the group consisting of L-carnitine and conjugated linoleic acid; and (b) said second stage pet food composition comprising, on a dry matter basis, about 20 to 35% by weight of a protein, about 4 to 10% by weight of a fat, about 2 to 25% by weight of a fiber, about 25 to 70% by weight of a carbohydrate, and about 0.1 to 2% by weight of a functional ingredient, wherein said functional ingredient is selected from the group consisting of L-carnitine and conjugated linoleic acid, wherein the protein content of the second stage pet food composition is about 10 to 45% less than the protein content of the first stage pet food composition.

In some embodiments, a pet food composition comprises: a live probiotic microorganism; about 0.05 to 5 wt % of leucine and/or about 0.005 to 1 wt % of one or more leucine metabolites; and about 0.0005 to 0.05 wt % of A sirtuin activator. In some embodiments, a pet food composition comprises: a live probiotic microorganism; at least about 50 mg of leucine and/or at least about 5 mg of one or more leucine metabolites by weight of the pet food composition; and at least about 1 mg of a sirtuin activator by weight of the pet food composition. In some embodiments, the probiotic microorganism is selected from the group consisting of Bifidobacterium, Bacteroides, Clostridium, Fusobacterium, Melissococcus, Propionibacterium, Streptococcus, Enterococcus, Lactococcus, Staphylococcus, Peptostrepococcus, Bacillus, Pediococcus, Micrococcus, Leuconostoc, Weissella, Aerococcus, Oenococcus, or Lactobaccillus. In some embodiments, said composition comprises a starch source. In some embodiments, said starch source has a degree of gelatinization less than about 7.5 Joules/g of starch.

In some embodiments, a pet food compositions comprises: about 0.05 to 5 wt % of leucine and/or about 0.005 to 1 wt % of one or more leucine metabolites; about 0.0005 to 0.05 wt % of a sirtuin activator; and a pre-selected balance of metabolizable cations to metabolizable anions, wherein the DCAB is between about 50 to 300. In some embodiments, a method for treating a puppy susceptible to or suffering from diarrhea and/or loose stool comprises: feeding the puppy a food composition comprising (i) about 0.05 to 5 wt % of leucine and/or about 0.005 to 1 wt % of one or more leucine metabolites and (ii) about 0.0005 to 0.05 wt % of a sirtuin activator; and adjusting the balance of metabolizable cations to metabolizable anions consumed by the puppy by an amount sufficient to improve stool quality by increasing the balance of metabolizable cations to metabolizable anions consumed by the puppy to produce firmer stool.

In some embodiments, a pet food composition for reducing odor of stool of a companion animal comprises: stool odor-reducing effective amount of ginger or an extract thereof; about 0.05 to 5 wt % of leucine and/or about 0.005 to 1 wt % of one or more leucine metabolites; and about 0.0005 to 0.05 wt % of a sirtuin activator. In some embodiments, said amount of ginger or extract thereof is present at an amount of at least about 500 mg. In some embodiments, said amount of ginger or extract thereof is about 0.005 to 12% by weight. In some embodiments, the method comprises causing the animal to ingest a pet food composition comprises: a stool odor-reducing effective amount of ginger or an extract thereof; about 0.05 to 5 wt % of leucine and/or about 0.005 to 1 wt % of one or more leucine metabolites; and about 0.0005 to 0.05 wt % of a sirtuin activator. In some embodiments, said amount of ginger or extract thereof is about 0.005 to 12% by weight. In some embodiments, said amount of ginger or extract thereof is present at an amount of at least about 500 mg.

In some embodiments, a pet food composition comprises: chicory pulp in an amount which: i) maintains good feces quality or improves the feces quality of a pet and/or ii) maintains good gastrointestinal tract health and/or improves the gastrointestinal tract health of a pet; about 0.05 to 5 wt % of leucine and/or about 0.005 to 1 wt % of one or more leucine metabolites; and about 0.0005 to 0.05 wt % of a sirtuin activator. In some embodiments, said chicory pulp comprises from 0.5 to 20% dry weight of the pet food product. In some embodiments, said chicory pulp comprises from 2 to 10% dry weight of the pet food product. In some embodiments, said chicory pulp is present at an amount of at least about 350 mg.

In some embodiments, a pet food composition comprises an inner layer comprising kibble; an outer layer; about 0.05 to 5% of leucine and/or about 0.005 to 1% of one or more leucine metabolites by weight of the pet food composition; and about 0.0005 to 0.05% of a sirtuin activator by weight of the pet food composition. In some embodiments, the inner layer comprises about 0.05 to 5% of leucine and/or about 0.005 to 1% of one or more leucine metabolites by weight of the pet food composition; and about 0.0005 to 0.05% of a sirtuin activator by weight of the pet food composition. In some embodiments, the outer layer comprises about 0.05 to 5% of leucine and/or about 0.005 to 1% of one or more leucine metabolites by weight of the pet food composition; and about 0.0005 to 0.05% of a sirtuin activator by weight of the pet food composition. In some embodiments, a pet food or supplement composition comprises an inner layer comprising kibble; an outer layer; and at least about 50 mg of leucine and/or at least about 5 mg of one or more leucine metabolites by weight of the pet food composition; and at least about 1 mg of a sirtuin activator by weight of the pet food composition. In some embodiments, the inner layer comprises at least about 50 mg of leucine and/or at least about 5 mg of one or more leucine metabolites by weight of the pet food composition; and at least about 1 mg of a sirtuin activator by weight of the pet food composition. In some embodiments, the outer layer comprises at least about 50 mg of leucine and/or at least about 5 mg of one or more leucine metabolites by weight of the pet food composition; and at least about 1 mg of a sirtuin activator by weight of the pet food composition.

In some embodiments, a pet food composition comprises about 0.05 to 5 wt % of leucine and/or about 0.005 to 1 wt % of one or more leucine metabolites; about 0.0005 to 0.05 wt % of a sirtuin activator; and a palatability enhancer selected from the group consisting of butyric acid, 3-methylbutyric acid, tetrasodium pyrophosphate, 2-piperidione, 2,3 pentanedione, 2-ethyl-3,5-dimethylpyrazine, furfural, sulfurol, and indole. In some embodiments, a pet food or supplement composition comprising at least about 50 mg of leucine and/or at least about 5 mg of one or more leucine metabolites by weight of the pet food composition; at least about 1 mg of a sirtuin activator by weight of the pet food composition; and a palatability enhancer selected from the group consisting of butyric acid, 3-methylbutyric acid, tetrasodium pyrophosphate, 2-piperidione, 2,3 pentanedione, 2-ethyl-3,5-dimethylpyrazine, furfural, sulfurol, and indole.

In some embodiments, a pet food composition comprises: an outer layer joined to an inner layer, wherein the outer layer is harder than the inner layer; about 0.05 to 5 wt % of leucine and/or about 0.005 to 1 wt % of one or more leucine metabolites; and about 0.0005 to 0.05 wt % of a sirtuin activator.

In some embodiments, a pet food composition comprises: a structurant for providing a textured appearance and feel; about 0.05 to 5 wt % of leucine and/or about 0.005 to 1 wt % of one or more leucine metabolites; and about 0.0005 to 0.05 wt % of a sirtuin activator. In some embodiments, a pet food composition comprises a textured layer bonded to a base layer, wherein the textured layer comprises textured components bonded to the base layer; about 0.05 to 5 wt % of leucine and/or about 0.005 to 1 wt % of one or more leucine metabolites; and about 0.0005 to 0.05 wt % of a sirtuin activator.

In some embodiments, a pet food composition comprises: less than about 19% on a dry weight basis of carbohydrate; about 0.05 to 5 wt % of leucine and/or about 0.005 to 1 wt % of one or more leucine metabolites; and about 0.0005 to 0.05 wt % of a sirtuin activator. In some embodiments, the pet food composition is dimensionally stable.

In some embodiments, a method for feeding a pet comprises: providing, over an extended and preselected period of time, different food compositions to said animal in which each composition provides an enriched source of fat, protein or carbohydrate, such that said animal can select and consume different and preferred quantities of each said food compositions in order to achieve an preferred consumption of fat, protein and carbohydrate for said animal; wherein at least one of said food compositions comprises about 0.05 to 5 wt % of leucine and/or about 0.005 to 1 wt % of one or more leucine metabolites; and about 0.0005 to 0.05 wt % of a sirtuin activator; allowing said animal to consume the different and preferred quantities of fat, protein and carbohydrate from each of said compositions over the extended preselected period of time; and determining, from the consumed amount of fat, protein and carbohydrate from each of said compositions, a customized dietary regime that provides the preferred macronutrient content of a diet for said individual animal.

In some embodiments, a computer-readable medium comprises code that, upon execution by one or more processors, implements a method of producing a customized dry pet food composition formulated from a dry pet food kibble recipe and selected functional ingredients, the formulation being selected on the basis of an individual pet's attributes and physical conditions, with the method comprising receiving information on the individual pet's attributes and physical conditions; selecting a predetermined volume of dry kibble pieces from a plurality different formulations of pre-made dry kibble pieces; selecting one or more functional ingredients from a plurality functional ingredients; coating the predetermined volume of dry kibble pieces with the one or more functional ingredients; and packaging and labeling the predetermined volume of coated dry kibble pieces; wherein the selection of the predetermined volume of dry kibble pieces and one or more functional ingredients is based on the individual pet's attributes and physical conditions to provide the customized dry pet food product, and wherein the coating and/or the kibble pieces comprise leucine and/or a leucine metabolite and a sirtuin activator. In some embodiments, the leucine, if present, is in an amount that is about 0.05 to 5 wt %; the leucine metabolites, if present, are in an amount that is about 0.005 to 1 wt %; and the a sirtuin activator, if present, is in an amount that is about 0.0005 to 0.05 wt %. In some embodiments, a method of producing a customized dry pet food product formulated from a dry pet food kibble recipe and selected functional ingredients, the formulation being selected on the basis of an individual pet's attributes and physical conditions, the method comprises: providing a plurality of different formulations of pre-made dry kibble pieces; selecting a predetermined volume of dry kibble pieces from the plurality of different formulations of pre-made dry kibble pieces; providing a plurality of functional ingredients; coating the volume of dry kibble pieces with one or more of the plurality of functional ingredients; and packaging and labeling the predetermined volume of coated dry kibble pieces; wherein the selection of the predetermined volume of dry kibble pieces and the one or more functional ingredients is based on the individual pet's attributes and physical conditions to provide the customized dry pet food product, and wherein the coating and/or the kibble pieces comprise leucine and/or a leucine metabolite and a sirtuin activator. In some embodiments, the leucine, if present, is in an amount that is about 0.05 to 5 wt %; the leucine metabolites, if present, are in an amount that is about 0.005 to 1 wt %; and the a sirtuin activator, if present, is in an amount that is about 0.0005 to 0.05 wt %.

In some embodiments, a diet for companion animals comprises: a first stage pet food composition and a second stage pet food composition for maintaining the weight loss, wherein each of said first stage pet food composition and said second stage pet food composition comprise, on a dry matter basis, about 0.05 to 5 wt % of leucine and/or about 0.005 to 1 wt % of one or more leucine metabolites, and about 0.0005 to 0.05 wt % of a sirtuin activator, and wherein said second stage pet food comprises at least about 5% higher fat content compared to said first stage pet food.

In some embodiments, a vegetarian pet food composition comprises: a vegetarian kibble which incorporates a non-meat based flavor-enhancing additive; about 0.05 to 5 wt % of leucine and/or about 0.005 to 1 wt % of one or more leucine metabolites; and about 0.0005 to 0.05 wt % of a sirtuin activator. In some embodiments, a vegetarian pet food or supplement composition comprises: a vegetarian kibble which incorporates a non-meat based flavor-enhancing additive; at least about 50 mg of leucine and/or at least about 5 mg of one or more leucine metabolites by weight of the pet food composition; and at least about 1 mg of a sirtuin activator by weight of the pet food composition.

In some embodiments, a multi-component pet food composition comprises two or more compartmentalized food compositions, wherein the at least two compartmentalized compositions differ in their content in at least two of fat, protein or carbohydrate, and further wherein one of the two or more compartmentalized compositions comprises about 0.05 to 5 wt % of leucine and/or about 0.005 to 1 wt % of one or more leucine metabolites, and about 0.0005 to 0.05 wt % of a sirtuin activator.

In some embodiments, a pet food composition for increasing the shelf life of a physically discrete dry pet food comprises: a coat covering the physically discrete pet food composition comprising a polymer film, wherein the film or an agent in the film protects the composition from oxidation decomposition and/or protects the composition from bacterial growth, wherein the film comprises a starch/synthetic polymer selected from the group consisting of starch/polyethylene, and starch/low-density polyethylene, and wherein the thickness of said coat is 1-2000 microns; about 0.05 to 5 wt % of leucine and/or about 0.005 to 1 wt % of one or more leucine metabolites; and about 0.0005 to 0.05 wt % of a sirtuin activator. In some embodiments, a method for increasing the shelf life of a physically discrete dry pet food composition comprises: coating the physically discrete pet food composition with a polymer film, wherein the film or an agent in the film protects the composition from oxidation decomposition and/or protects the composition from bacterial growth, wherein the film comprises a starch/synthetic polymer selected from the group consisting of starch/polyethylene, and starch/low-density polyethylene, wherein the thickness of said coating is 1-2000 microns; and wherein the pet food composition comprises about 0.05 to 5 wt % of leucine and/or about 0.005 to 1 wt % of one or more leucine metabolites and about 0.0005 to 0.05 wt % of a sirtuin activator.

In some embodiments, method for making kibble at one location and finishing the pet food at another location comprises: forming a dry, stable intermediate of the pet food composition at a first location; finishing the dry, stable intermediate pet food composition to form a finished pet food composition at a second location remote from the first location, wherein said finished pet food composition contains about 0.05 to 5 wt % of leucine and/or about 0.005 to 1 wt % of one or more leucine metabolites and about 0.0005 to 0.05 wt % of a sirtuin activator.

In some embodiments, a hypoallergenic pet food composition comprises: proteinaceous component that has been hydrolyzed whereby said component is rendered hypoallergenic to a pet, wherein said proteinaceous component is made up of polypeptides and free amino acids having an average molecular weight of less than about 3,000 Daltons; about 0.05 to 5 wt % of leucine and/or about 0.005 to 1 wt % of one or more leucine metabolites; and about 0.0005 to 0.05 wt % of a sirtuin activator.

In some embodiments, a method of administering a pet food, treat, supplement, or drink to a pet comprises administering a composition to the pet. In some embodiments, a method of facilitating weight loss in a pet in need thereof comprising administering a composition to the pet, wherein the pet experiences weight loss. In some embodiments, the pet loses about 10% of its weight relative to the weight of the pet prior to administration of the composition. In some embodiments, a method of facilitating fat loss in a pet in need thereof comprises administering a composition to the pet, wherein the pet experiences fat loss. In some embodiments, the pet loses about 10% fat relative to the amount of fat prior to administration of the composition. In some embodiments, a method of reducing body condition score of a pet in need thereof comprises administering a composition to the pet, wherein the pet experiences a reduction in body condition score. In some embodiments, the pet's body condition score is reduced by about 2 relative to the pet's body condition score prior to administration of the composition.

In some embodiments, a method of increasing production of irisin in a pet comprises administering to the pet a pet food, pet treat, pet snack or pet drink composition that comprises: about 0.05 to 5 wt % of leucine and/or about 0.005 to 1 wt % of one or more leucine metabolites; and about 0.0005 to 0.05 wt % of a sirtuin activator, wherein the production of irisin in the pet is increased. In some embodiments, a method of increasing production of irisin in a pet comprises administering to the pet a pet food, pet treat, pet snack or pet drink composition that comprises: at least about 50 mg of leucine and/or at least about 5 mg of one or more leucine metabolites; about 1 mg of a sirtuin activator, wherein the production of irisin in the pet is increased.

In some embodiments, a method of increasing insulin sensitivity in a pet comprises administering to the pet a pet food, pet treat, pet snack or pet drink composition that comprises: about 0.05 to 5 wt % of leucine and/or about 0.005 to 1 wt % of one or more leucine metabolites; and about 0.0005 to 0.05 wt % of a sirtuin activator, wherein the insulin sensitivity of the pet is increased. In some embodiments, a method of increasing insulin sensitivity in a pet comprises administering to the pet a pet food, pet treat, pet snack or pet drink composition that comprises: at least about 50 mg of leucine and/or at least about 5 mg of one or more leucine metabolites; at least about 1 mg of a sirtuin activator, wherein the insulin sensitivity in the pet is increased.

In some embodiments, a method of reducing inflammation in a pet comprises administering to the pet a pet food, pet treat, pet snack or pet drink composition that comprises: about 0.05 to 5 wt % of leucine and/or about 0.005 to 1 wt % of one or more leucine metabolites; and about 0.0005 to 0.05 wt % of a sirtuin activator, wherein the inflammation in the pet is reduced. In some embodiments, a method reducing inflammation in a pet comprises administering to the pet a pet food, pet treat, pet snack or pet drink composition that comprises: at least about 50 mg of leucine and/or at least about 5 mg of one or more leucine metabolites; and about 1 mg of a sirtuin activator, wherein the inflammation in the pet is reduced.

In some embodiments, a method of reducing and/or preventing diabetes in a pet comprises administering a pet food, pet treat, pet snack or pet drink composition that comprises: about 0.05 to 5 wt % of leucine and/or about 0.005 to 1 wt % of one or more leucine metabolites; and about 0.0005 to 0.05 wt % of a sirtuin activator, wherein the effects of diabetes in the pet are reduced or prevented. In some embodiments, a method of reducing and/or preventing diabetes in a pet comprises administering a pet food, pet treat, pet snack or pet drink composition that comprises: at least about 50 mg of leucine and/or at least about 5 mg of one or more leucine metabolites; and at least about 1 mg of a sirtuin activator, wherein the effects of diabetes in the pet are reduced or prevented.

In some embodiments, a pet food composition comprises: about 0.05 to 5 wt % of leucine and/or about 0.005 to 1 wt % of one or more leucine metabolites; and about 0.0005 to 0.05 wt % of a sirtuin activator; and about 1.5 g/kg metformin, about 0.75 g/kg metformin, about 0.25 g/kg metformin. In some embodiments, a pet food, treat or supplement composition comprises: at least about 50 mg of leucine and/or at least about 5 mg of one or more leucine metabolites; at least about 1 mg of a sirtuin activator; and at least about 125 mg metformin

In some embodiments, the leucine metabolite is selected from the group consisting of keto-isocaproic acid (KIC), alpha-hydroxy-isocaproic acid, and hydroxymethylbutyrate (HMB). In some embodiments, the sirtuin pathway activator is a hydroxycinnamic acid or a stilbene. In some embodiments, the sirtuin pathway activator is resveratrol. In some embodiments, the composition is substantially free of alanine in free form or salt form. In some embodiments, the composition is substantially free of glutamic acid in free form or salt form. In some embodiments, the composition is substantially free of glycine in free form or salt form. In some embodiments, the composition is substantially free of proline in free form or salt form. In some embodiments, the composition is substantially free of alanine in free form or salt form. In some embodiments, the composition is substantially free of non-leucine amino acids in free of salt form.

In some embodiments, the composition comprises less than 1% of alanine in free form or salt form. In some embodiments, the composition comprises less than 1% of glutamic acid in free form or salt form. In some embodiments, the composition comprises less than 1% of glycine in free form or salt form. In some embodiments, the composition comprises less than 1% of proline in free form or salt form. In some embodiments, the composition comprises less than 1% of non-leucine amino acids in free form or salt form.

In some embodiments, the amount of leucine is between about 50-400, 50-300, 50-250, or 50-200 mg. In some embodiments, the amount of leucine metabolites is between about 5-50, 5-25, or 5-10 mg. In some embodiments, the amount of sirtuin activator is between about 1-10, 1-5, or 1-2 mg.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 depicts a diagram showing a sirtuin pathway.

FIG. 2 shows a non-limiting example of a computer system useful in the methods of the invention.

### DETAILED DESCRIPTION OF THE INVENTION

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

An “activator” refers to a modulator that influences a pathway in a manner that increases the pathway output. Activation of a particular target may be direct (e.g. by interaction with the target) or indirect (e.g. by interaction with a protein upstream of the target in a signaling pathway including the target).

The terms “administer”, “administered”, “administers” and “administering” are defined as the providing a composition to a subject via intravenous, intraarterial, oral, parenteral, buccal, topical, transdermal, rectal, intramuscular, subcutaneous, intraosseous, transmucosal, or intraperitoneal routes of administration. In certain embodiments of the subject application, oral routes of administering a composition may be preferred.

As used herein, “agent” or “biologically active agent” refers to a biological, pharmaceutical, or chemical compound or other moiety. Non-limiting examples include simple or complex organic or inorganic molecule, a peptide, a protein, a peptide nucleic acid (PNA), an oligonucleotide (including e.g., aptomer and polynucleotides), an antibody, an antibody derivative, antibody fragment, a vitamin derivative, a carbohydrate, a toxin, or a chemotherapeutic compound. Various compounds can be synthesized, for example, small molecules and oligomers (e.g., oligopeptides and oligonucleotides), and synthetic organic compounds based on various core structures. In addition, various natural sources can provide compounds for screening, such as plant or animal extracts, and the like. A skilled artisan can readily recognize that there is no limit as to the structural nature of the agents of the present invention.

As used herein, the terms “animal” or “pet” mean a domestic animal including, but not limited to domestic dogs, cats, horses, cows, ferrets, rabbits, pigs, rats, mice, gerbils, hamsters, horses, and the like. Domestic dogs and cats are particular examples of pets.

As used herein, the terms “animal feed”, “animal feed compositions”, “animal feed kibble”, “pet food”, or “pet food composition” all mean a composition intended for ingestion by a pet. Pet foods may include, without limitation, nutritionally balanced compositions suitable for daily feed, such as kibbles, as well as supplements and/or treats, which may or, may not be nutritionally balanced.

The term “effective amount” or “therapeutically effective amount” refers to that amount of an inhibitor described herein that is sufficient to effect the intended application including but not limited to disease treatment, as defined below. The therapeutically effective amount may vary depending upon the intended application (in vitro or in vivo), or the subject and disease condition being treated, e.g., the weight and age of the subject, the severity of the disease condition, the manner of administration and the like, which can readily be determined by one of ordinary skill in the art. The term also applies to a dose that will induce a particular response in target cells, e.g., reduction of proliferation or down regulation of activity of a target protein. The specific dose will vary depending on the particular compounds chosen, the dosing regimen to be followed, whether it is administered in combination with other compounds, timing of administration, the tissue to which it is administered, and the physical delivery system in which it is carried.

The term “energy metabolism,” as used herein, refers to the transformation of energy that accompanies biochemical reactions in the body, including cellular metabolism and mitochondrial biogenesis. Energy metabolism can be quantified using the various measurements described herein, for example, weight-loss, fat-loss, insulin sensitivity, fatty acid oxidation, glucose utilization, triglyceride content, Sirt 1 expression level, AMPK expression level, oxidative stress, and mitochondrial biomass.

A “modulator” of a pathway refers to a substance or agent which modulates the activity of one or more cellular proteins mapped to the same specific signal transduction pathway. A modulator may augment or suppress the activity and/or expression level or pattern of a signaling molecule. A modulator can activate a component in a pathway by directly binding to the component. A modulator can also indirectly activate a component in a pathway by interacting with one or more associated components. The output of the pathway can be measured in terms of the expression or activity level of proteins. The expression level of a protein in a pathway can be reflected by levels of corresponding mRNA or related transcription factors as well as the level of the protein in a subcellular location. For instance, certain proteins are activated by translocating in or out of a specific subcellular component, including but not limited to nucleus, mitochondria, endosome, lysosome or other membranous structure of a cell. The output of the pathway can also be measured in terms of physiological effects, such as mitochondrial biogenesis, fatty acid oxidation, or glucose uptake.

A “sub-therapeutic amount” of an agent or therapy is an amount less than the effective amount for that agent or therapy, but when combined with an effective or sub-therapeutic amount of another agent or therapy can produce a result desired by the physician, due to, for example, synergy in the resulting efficacious effects, or reduced side effects.

“Subject” refers to animals, including domesticated animals and agricultural animals. The methods described herein can be useful in veterinary applications. In some embodiments, the subject is a mammal, including apes, chimpanzees, orangutans, monkeys; domesticated animals (pets) such as dogs, cats, guinea pigs, hamsters, mice, rats, rabbits, and ferrets; domesticated farm animals such as cows, buffalo, bison, horses, donkey, swine, sheep, and goats; or exotic animals typically found in zoos, such as bear, lions, tigers, panthers, elephants, hippopotamus, rhinoceros, giraffes, antelopes, sloth, gazelles, zebras, wildebeests, prairie dogs, koala bears, kangaroo, pandas, giant pandas, hyena, seals, sea lions, and elephant seals.

The term “substantially free,” as used herein, refers to compositions that have less than about 10%, less than about 5%, less than about 1%, less than about 0.5%, less than 0.1% or even less of a specified component. For example a composition that is substantially free of non-branched chain amino acids may have less than about 1% of non-branched chain amino acids.

A “suppressor” can be a modulator that influences a pathway in a manner that decreases pathway output.

Compositions

The present invention provides for systems, compositions, methods, and kits for promoting and for maintaining weight loss in companion animals. The systems, compositions, methods, and kits can avoid utilizing severe calorie restriction or caloric dilution, and the problems associated with such diets. The present invention also provides for a weight management system for domestic animals that allows for the selection of a desired animal body composition using a nutritionally complete pet food product.

The pet food of the present invention can be formulated to provide the daily nutritional and caloric requirements of an animal without a prescription. Feeding the prescription-free comprehensive weight management system of the subject invention to companion animals can help a domestic animal achieve a desired body composition by modulating metabolism, decreasing body fat, and/or building lean body mass.

The subject compositions include pet foods (including pet treats), pet supplements, and pet drinks. The subject compositions can be formulated to include active ingredients that promote weight loss in pets, including leucine, a leucine metabolite, and a sirtuin activator. The subject pet food compositions can provide a domestic animal with its daily nutritional requirements, or can be a treat or snack that is occasionally fed to a pet, and may not be designed to provide the pet with its daily nutritional requirements.

The compositions can be comprise: an effective amount of (a) leucine and/or one or more metabolites thereof, and (b) a sirtuin activator, wherein the combination when administered to a subject in need thereof enhances energy metabolism, including cellular metabolism, and mitochondrial biogenesis. The composition, when administered to a subject in need thereof, can enhance energy metabolism, including cellular metabolism and mitochondrial biogenesis, as measured by a decrease in weight gain of a subject, a decrease in adipose volume of a subject, an increase in fat oxidation of a subject, an increase in insulin sensitivity of a subject, a decrease in oxidative stress markers of a subject, and/or a decrease in inflammatory markers of a subject. In some embodiments, the composition is substantially free of free or individual non-branched chain amino acids or non-leucine amino acids.

The enhanced energy metabolism can be quantified by an increase in weight loss of a subject by at least 5, 10, 30, or 40%, a decrease in weight of about 1, 2, or 3 kg, a decrease in body condition score of at least about 1, 2 or 3, an increase in fat loss of a subject by at least about 1, 5, 10, 20, 30, or 50%, or an increase in insulin sensitivity by at least about 1, 5, 10, or 15% when the composition is administered to the subject. The enhanced energy metabolism can be measured relative to the dosing of the subject with a placebo, or relative to the subject prior to administration of the subject composition.

In some embodiments, the body condition score is on a scale of 1 to 9, where scores of 1-3 are represent a body condition that is too thin, 4-5 is ideal, and 6-8 is too heavy. The 1 to 9 scale can be based on that developed by Purina, where 1=Ribs, lumbar vertebrae, pelvic bones and all bony prominences evident from a distance. No discernible body fat. Obvious loss of muscle mass; 2=Ribs, lumbar vertebrae and pelvic bones easily visible. No palpable fat. Some evidence of other bony prominence. Minimal loss of muscle mass; 3=Ribs easily palpated and may be visible with no palpable fat. Tops of lumbar vertebrae visible. Pelvic bones becoming prominent. Obvious waist and abdominal tuck.; 4=Ribs easily palpable, with minimal fat covering. Waist easily noted, viewed from above. Abdominal tuck evident.; 5=Ribs palpable without excess fat covering. Waist observed behind ribs when viewed from above. Abdomen tucked up when viewed from side; 6=Ribs palpable with slight excess fat covering. Waist is discernible viewed from above but is not prominent. Abdominal tuck apparent; 7=Ribs palpable with difficulty; heavy fat cover. Noticeable fat deposits over lumbar area and base of tail. Waist absent or barely visible. Abdominal tuck may be present; 8=Ribs not palpable under very heavy fat cover, or palpable only with significant pressure. Heavy fat deposits over lumbar area and base of tail. Waist absent. No abdominal tuck. Obvious abdominal distention may be present.; 9=Massive fat deposits over thorax, spine and base of tail. Waist and abdominal tuck absent. Fat deposits on neck and limbs. Obvious abdominal distention.

Alternatively, the body condition score can be on a scale of 1 to 5, where 1=emaciated, 2=thin, 3=moderate, 4=stout, and 5=obese.

In other embodiments, the subject compositions can have an effect that is about 50, 75, 90, 100, 110, or 125% of the effect of a prescription weight loss diet, where the effect is an increase in weight loss, a decrease in weight, a decrease in body condition score, an increase in fat loss, or an increase in insulin sensitivity.

Leucine and Leucine Metabolites

The invention provides for compositions that include leucine and/or leucine metabolites. The leucine and/or leucine metabolites can be used in free form. The term “free,” as used herein in reference to a component, indicates that the component is not incorporated into a larger molecular complex. For example a composition can include free leucine that is not incorporated in a protein or free hydroxymethylbutyrate. The leucine can be L-leucine.

Without being limited to theory, ingestion of branched chain amino acids, such as leucine, can stimulate tissue protein synthesis via both mTOR-dependent and -independent pathways, as well as to exert an antiproteolytic effect. These effects predominate in muscle, but also can manifest in other tissues, including adipose tissue. Given the energetic cost of protein synthesis and turnover, leucine may increase fatty acid oxidation and net energy utilization and attenuate adiposity. Indeed, leucine has been reported to exert a thermogenic effect and to augment weight and adipose tissue loss during energy restriction. Also, leucine and leucine-rich diets to favorably modulate inflammatory cytokine patterns in adipocytes and mice.

In some embodiments, any of the compositions described herein can include salts, derivatives, metabolites, catabolites, anabolites, precursors, and analogs of any of the branched chain amino acids, such as a leucine salt. For example, the metabolites can include hydroxymethylbutyrate (HMB), α-hydroxyisocaproic acid, and keto-isocaproic acid (KIC), keto isovalerate, and keto antelisocaproate. Non-limiting exemplary anabolites of branched chain amino acids can include glutamate, glutamine, threonine, α-ketobytyrate, α-aceto-α-hydroxy butyrate, α,β-dihydroxy-β-methylvalerate, α-keto-β-methylvalerate, α,β-dihydroxy isovalerate, and α-keto isovalerate. The metabolites can include hydroxymethylbutyrate (HMB), keto-isocaproic acid (KIC), and keto isocaproate. The HMB can be in a variety of forms, including calcium 3-hydroxy-3-methylbutyrate hydrate. For clarity, the branched chain amino acids, leucine, and metabolites thereof, and other related compositions can be in free or individual form.

In some embodiments, the compositions may be substantially free of one or more, or all non-leucine amino acids. For example, the compositions can be free of alanine, arginine, asparagine, aspartic acid, cysteine, glutamic acid, glutamine, glycine, histidine, isoleucine, lysine, methionine, phenylalanine, proline, serine, threonine, tryptophan, tyrosine, and/or valine.

In some embodiments, the compositions may be substantially free of one or more, or all of non-branched chain amino acids. For example, the compositions can be substantially free of individual amino acids such as alanine, arginine, asparagine, aspartic acid, cysteine, glutamic acid, glutamine, glycine, histidine, lysine, methionine, phenylalanine, proline, serine, threonine, tryptophan, and/or tyrosine. The compositions can be substantially free of free amino acids such as alanine, arginine, asparagine, aspartic acid, cysteine, glutamic acid, glutamine, glycine, histidine, isoleucine, lysine, methionine, phenylalanine, proline, serine, threonine, tryptophan, tyrosine, and/or valine. The subject compositions can be substantially free of the individual amino acids alanine, glycine, glutamic acid, and proline. The subject compositions can be substantially free of one or more of the individual amino acids alanine, glycine, glutamic acid, and proline. The subject compositions can be substantially free of alanine. The subject compositions can be substantially free of glycine. The subject compositions can be substantially free of valine. The subject compositions can comprise less than 10, 5, 1, or 0.1% of the individual amino acids alanine, glycine, glutamic acid, and proline. For clarity, the non-branched amino acids described herein are intact amino acids existing in free form or salt form thereof. For example, the subject compositions can be substantially free of free amino acids, such as alanine, glycine, glutamic acid, and proline. The mass or molar amount of a non-branched chain amino acid, any amino acid, or any non-leucine amino acid can be less than about 0.01, 0.1, 0.5, 1, 2, 5, or 10% of the total composition, of the total amino acids in the composition, or of the total free amino acids in the composition.

A subject composition, which may be a pet food, treat, snack, supplement, or drink, can comprise at least about, about, or more than about 0.005, 0.01, 0.05, 0.075, 0.1, 0.25, 0.5, 1, 2.5, or 5 wt % of leucine, as a percent of the total composition, or any component of the total composition. A subject composition, which may be a pet food, treat, snack, supplement, or drink, can comprise between about 0.005-5, 0.005-1, 0.01-5, 0.05-5, 0.05-2, 0.05-1, 0.05-0.5, 0.1-5, or 0.5-2 wt % of leucine, as a percent of the total composition, or any component of the total composition.

A subject composition, which may be a pet food, treat, snack, supplement, or drink, can comprise at least about, about, or more than about 0.0005, 0.001, 0.005, 0.01, 0.05, 0.075, 0.1, 0.25, 0.5, 1, 2.5, or 5 wt % of one or more leucine metabolites, as a percent of the total composition, or any component of the total composition. A subject composition, which may be a pet food, treat, snack, supplement, or drink, can comprise between about 0.0005-5, 0.001-5, 0.001-1, 0.005-1, 0.005-0.5, 0.005-0.05 wt % of one or more leucine metabolites, as a percent of the total composition, or any component of the total composition.

A subject composition, which may be a pet food, treat, snack, supplement, or drink, can comprise about, more than about, or less than about 10, 20, 30, 50, 70, 100, 150, 200, 250, 400, 500, 600, 700, 800, 900, 1000, 1100, 1250, or more mg of leucine. The leucine may be free leucine. In some embodiments, a unit dose can comprise at least about 50 mg of free leucine. The composition may comprise between about 10-1250, 10-100, 30-100, 40-150, 200-1250, or 500-1250 mg of leucine. A subject composition, which may be a pet food, treat, snack, supplement, or drink, can comprise about, more than about, or less than about 0.1, 1, 2, 3, 4, 5, 7, 10, 20, 30, 50, 100, 200, 250, 400, 500, 600, 700, 800, 900, 1000 or more mg of a leucine metabolite, such as HMB or KIC. The leucine metabolite may be a free leucine metabolite. The composition may comprise between about 0.1-10, 1-10, 3-20, 5-200, 10-900, 50-750, or 400-650 mg of the leucine metabolite, such as HMB or KIC. In some embodiments, a unit dose can comprise at least about 100 mg of free HMB.

In some embodiments, a daily dose of leucine can be about, less than about, or more than about 0.005 g/day (e.g. 0.005, 0.01, 0.05, 0.1, 0.5, 0.75, 1, 1.25, 1.5, 1.75, 2, 2.5, 3, or more g/day). A daily dose of HMB or alpha-hydroxy-isocaproic acid can be about, less than about, or more than about 0.0001 g/day (e.g. 0.0001, 0.0005, 0.001, 0.005, 0.01, 0.05, 0.1, 0.2, 0.4, 0.5, 0.75, 1, 1.5, 2, 2.5, 3, or more g/day). A daily dose of KIC can be about, less than about, or more than about 0.005 g/day (e.g. 0.005, 0.01, 0.05, 0.1, 0.2, 0.4, 0.5, 0.75, 1, 1.25, 1.5, 1.75, 2, 2.5, 3, or more g/day).

In some embodiments, the dosing of leucine, any metabolites of leucine, can be designed to achieve a specified physiological concentration or circulating level of leucine and/or metabolites of leucine. The physiological concentration can be a circulating level as measured in the blood stream of a subject. The subject can be a human or an animal, such as a pet. The amount of leucine in a unit dose can be such that the circulating level of leucine in a subject, such as a pet, is about or greater than about 0.25 mM, 0.5 mM, 0.75 mM, or 1 mM.

The amount of leucine in a unit dose, such as a pet food meal, a pet snack, or a pet supplement can be such that the circulating level of leucine in the pet is about or greater than about 0.25 mM, 0.5 mM, 0.75 mM, or 1 mM. A dosing of about 1,125 mg leucine can achieve a circulating level of leucine in a subject weighing about 150 lb that is about 0.5 mM. A dosing of about 300 mg leucine can achieve a circulating level of leucine in a subject weighing about 150 lb that is about 0.25 mM. Correspondingly, A dosing of about 225 mg leucine can achieve a circulating level of leucine in a subject weighing about 30 lb that is about 0.5 mM. A dosing of about 60 mg leucine can achieve a circulating level of leucine in a subject weighing about 30 lb that is about 0.25 mM.

Sirtuin Activators

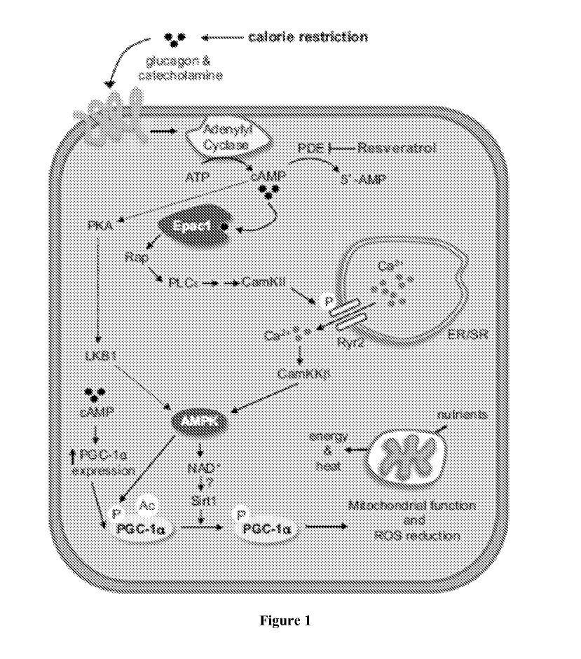

The invention provides for compositions that can increase or modulate the output of a sirtuin pathway. Sirtuin activators are described in U.S. patent Ser. Nos. 13/549,381 and 13/549,399, which are hereby incorporated by reference in their entirety. The sirtuin pathway includes, without limitation, signaling molecules such as, Sirt1, Sirt3, and AMPK. The output of the pathway can be determined by the expression level and/or the activity of the pathway and/or a physiological effect. In some embodiments, activation of the Sirt1 pathway includes stimulation of PGC1-α and/or subsequent stimulation of mitochondrial biogenesis and fatty acid oxidation. In general, a sirtuin pathway activator is compound that activates or increases one or more components of a sirtuin pathway. An increase or activation of a sirtuin pathway can be observed by an increase in the activity of a pathway component protein. For example, the protein can be Sirt1, PGC1-α, AMPK, Epac1, Adenylyl cyclase, Sirt3, or any other proteins and their respective associated proteins along the signaling pathway depicted in FIG. 1 (Park et. al., “Resveratrol Ameliorates Aging-Related Metabolic Phenotypes by Inhibiting cAMP Phosphodiesterases,” Cell 148, 421-433 Feb. 3, 2012). Non-limiting examples of physiological effects that can serve as measures of sirtuin pathway output include mitochondrial biogenesis, fatty acid oxidation, glucose uptake, palmitate uptake, oxygen consumption, carbon dioxide production, weight loss, heat production, visceral adipose tissue loss, respiratory exchange ratio, insulin sensitivity, inflammation marker level, vasodilation, browning of fat cells, and irisin production. Examples of indicia of browning of fat cells include, without limitation, increased fatty acid oxidation, and expression of one or more brown-fat-selective genes (e.g. Ucp1, Cidea, Prdm16, and Ndufs1). In some embodiments, changes in one or more physiological effects that can serve as measures of sirtuin pathway output are induced by increasing irisin production, such as by administering a composition of the invention.

An increase in mitochondrial biogenesis can be evidenced by an increase in the formation of new mitochondria and/or by an increase in mitochondrial functions, such as increased fatty acid oxidation, increased heat generation, increased insulin sensitivity, increased in glucose uptake, increased in vasodilation, decreased in weight, decreased in adipose volume, and decreased inflammatory response or markers in a subject.

The compositions can be combination compositions which may include one or more synergistic components. In some embodiments, the synergistic effect of the combination compositions can allow for reduced dosing amounts, leading to reduced side effects to the subject and reduced cost of treatment. In other embodiments, the synergistic effect can allow for results that are not achievable through any other conventional treatments. The subject combination compositions provide a significant improvement in the regulation of energy metabolism.

In some embodiments, any of the compositions described herein can include salts, derivatives, metabolites, catabolites, anabolites, precursors, and analogs of any of the forms of a sirtuin activator. The forms can be in free form, individual form, or salt form.

A subject composition, which may be a pet food, treat, snack, supplement, or drink, can comprise at least about, about, or more than about 0.0001, 0.0005, 0.001, 0.005, 0.01, 0.05, 0.075, 0.1, 0.25, 0.5, 1, 2.5, 5, or 10 wt % of a sirtuin activator, as a percent of the total composition, or any component of the total composition. A subject composition, which may be a pet food, treat, snack, supplement, or drink, can comprise between about 0.0001-0.05, 0.0001-0.005, 0.0005-0.05, 0.001-0.05, 0.001-0.01, 0.005-0.01, 0.001-2, 0.001-5, or 0.001-10 wt % of a sirtuin activator, as a percent of the total composition, or any component of the total composition.

A subject composition, which may be a pet food, treat, snack, supplement, or drink, can comprise about, more than about, or less than about 0.1, 0.2, 0.5, 0.7, 1, 2, 3, 4, 5, 10, 20, 30, 50, 70, 100, 150, 200, 250, 400, 500, 600, 700, 800, 900, 1000, 1100, 1250, or more mg of a sirtuin activator. The a sirtuin activator may be free a sirtuin activator. In some embodiments, a unit dose can comprise at least about 1 mg of free a sirtuin activator. The composition may comprise between about 0.1-125, 0.1-10, 1-50, 1-25, 3-10, 4-15, 20-125, or 50-125 mg of a sirtuin activator.

The amount of a sirtuin activator in a unit dose can be such that the circulating level of a sirtuin activator in a subject is about or greater than about 10, 25, 50, 100, 150, or 200 nM. The amount of leucine and a sirtuin activator in a unit dose can be such that the circulating level of leucine in a subject is about 100 nM.

A dosing of about 15 mg of a sirtuin activator can achieve a circulating level of a sirtuin activator in as subject weighing about 150 lb that is about 100 nM. A dosing of about 7.5 mg of a sirtuin activator can achieve a circulating level of a sirtuin activator in a subject weighing about 150 lb that is about 50 nM. Correspondingly, a dosing of about 3 mg of a sirtuin activator can achieve a circulating level of a sirtuin activator in as subject weighing about 30 lb that is about 100 nM. A dosing of about 1.5 mg of a sirtuin activator can achieve a circulating level of a sirtuin activator in a subject weighing about 30 lb that is about 50 nM.

An oral dosing of about 1100 mg of resveratrol in a subject weight about 150 lb can achieve a circulating level of resveratrol in the subject that is about 0.5 mM resveratrol. An oral dosing of about 50 mg of resveratrol to a subject weight about 150 lb can achieve a circulating level of resveratrol in the subject that is about 200 nM resveratrol. Correspondingly, an oral dosing of about 220 mg of resveratrol in a subject weight about 30 lb can achieve a circulating level of resveratrol in the subject that is about 0.5 mM resveratrol. An oral dosing of about 10 mg of resveratrol to a subject weight about 30 lb can achieve a circulating level of resveratrol in the subject that is about 200 nM resveratrol.

In some embodiments, the sirtuin-pathway activator or AMPK pathway activator can be a polyphenol. For example, the polyphenol can be chlorogenic acid, resveratrol, caffeic acid, piceatannol, ellagic acid, epigallocatechin gallate (EGCG), grape seed extract, or any analog thereof. In some embodiments, the activator can be resveratrol, an analog thereof, or a metabolite thereof. For example, the activator can be pterostilbene or a small molecule analog of resveratrol. Examples of small molecule analogs of resveratrol are described in U.S. Patent Application Nos. 20070014833, 20090163476, and 20090105246, which are incorporated herein by reference in its entirety.

The polyphenol can be a substantially homogeneous population of polyphenols. The polyphenol can be one type of polyphenol, wherein the composition can exclude all other types of polyphenols. In other embodiments, the composition can comprise two, three, or four types of polyphenols, and exclude all other types of polyphenols. In some embodiments, the composition can comprise 1, 2, 3, or 4 types of polyphenols and less than 0.1, 0.5, 1, or 2% of any other types of polyphenols. In some embodiments, a composition further comprises a phosphodiesterase (PDE) inhibitor, and/or other sirtuin pathway activator.

In various other embodiments, compositions are formulated such that they do not contain (or exclude) one or more of the following ingredients: caffeine, green tea extract or extracts from guarana seed or guarana plants.

In some embodiments, the sirtuin-pathway activator can be a hydroxycinnamic acid or a stilbene.

In other embodiments, the sirtuin-pathway activator or AMPK pathway activator can be irisin, quinic acid, cinnamic acid, ferulic acid, fucoxanthin, a biguanide (such as metformin), rosiglitazone, or any analog thereof. Alternatively the sirtuin-pathway activator or AMPK pathway activator can be isoflavones, pyroloquinoline (PQQ), quercetin, L-carnitine, lipoic acid, coenzyme Q10, pyruvate, 5-aminoimidazole-4-carboxamide ribotide (ALCAR), bezfibrate, oltipraz, and/or genistein. In some embodiments, the sirtuin pathway activator is a PDE inhibitor.

In some embodiments, the composition can comprise synergistic combinations of sirtuin pathway activators. For example, a composition can comprise synergistic amounts of metformin and a PDE inhibitor. In some embodiments, the composition comprises metformin and caffeine.

In some embodiments, the sirtuin-pathway activator can be an agent that stimulates the expression of the Fndc5, PGC1-α, or UCP1. The expression can be measured in terms of the gene or protein expression level. Alternatively, the sirtuin pathway activator can be irisin. Methods for increasing the level of irisin are described in Boström et al., “A PGC1-α-dependent myokine that drives brown-fat-like development of white fat and thermogenesis,” Nature, Jan. 11, 2012.

In some embodiments, the activator is a flavones or chalcone. In one embodiment, exemplary sirtuin activators are those described in Howitz et al. (2003) Nature 425: 191 and include, for example, resveratrol (3,5,4′-Trihydroxy-trans-stilbene), butein (3,4,2′,4′-Tetrahydroxychalcone), piceatannol (3,5,3′,4′-Tetrahydroxy-trans-stilbene), isoliquiritigenin (4,2′,4′-Trihydroxychalcone), fisetin (3,7,3′,4′-Tetrahyddroxyflavone), quercetin (3,5,7,3′,4′-Pentahydroxyflavone), Deoxyrhapontin (3,5-Dihydroxy-4′-methoxystilbene 3-O-β-D-glucoside); trans-Stilbene; Rhapontin (3,3′,5-Trihydroxy-4′-methoxystilbene 3-O-β-D-glucoside); cis-Stilbene; Butein (3,4,2′,4′-Tetrahydroxychalcone); 3,4,2′4′6′-Pentahydroxychalcone; Chalcone; 7,8,3′,4′-Tetrahydroxyflavone; 3,6,2′,3′-Tetrahydroxyflavone; 4′-Hydroxyflavone; 5,4′-Dihydroxyflavone 5,7-Dihydroxyflavone; Morin (3,5,7,2′,4′-Pentahydroxyflavone); Flavone; 5-Hydroxyflavone; (−)-Epicatechin (Hydroxy Sites: 3,5,7,3′,4′); (−)-Catechin (Hydroxy Sites: 3,5,7,3′,4′); (−)-Gallocatechin (Hydroxy Sites: 3,5,7,3′,4′,5′) (+)-Catechin (Hydroxy Sites: 3,5,7,3′,4′); 5,7,3′,4′,5′-pentahydroxyflavone; Luteolin (5,7,3′,4′-Tetrahydroxyflavone); 3,6,3′,4′-Tetrahydroxyflavone; 7,3′,4′,5′-Tetrahydroxyflavone; Kaempferol (3,5,7,4′-Tetrahydroxyflavone); 6-Hydroxyapigenin (5,6,7,4′-Tetrahydoxyflavone); Scutellarein); Apigenin (5,7,4′-Trihydroxyflavone); 3,6,2′,4′-Tetrahydroxyflavone; 7,4′-Dihydroxyflavone; Daidzein (7,4′-Dihydroxyisoflavone); Genistein (5,7,4′-Trihydroxyflavanone); Naringenin (5,7,4′-Trihydroxyflavanone); 3,5,7,3′,4′-Pentahydroxyflavanone; Flavanone; Pelargonidin chloride (3,5,7,4′-Tetrahydroxyflavylium chloride); Hinokitiol (b-Thujaplicin; 2-hydroxy-4-isopropyl-2,4,6-cycloheptatrien-1-one); L-(+)-Ergothioneine ((S)-α-Carboxy-2,3-dihydro-N,N,N-trimethyl-2-thioxo-1H-imidazole-4-ethanaminium inner salt); Caffeic Acid Phenyl Ester; MCI-186 (3-Methyl-1-phenyl-2-pyrazolin-5-one); HBED (N,N′-Di-(2-hydroxybenzyl)ethylenediamine-N,N′-diacetic acid-H2O); Ambroxol (trans-4-(2-Amino-3,5-dibromobenzylamino)cyclohexane-HCl; and U-83836E ((−)-2-((4-(2,6-di-1-Pyrrolidinyl-4-pyrimidinyl)-1-piperzainyl)methyl)-3,4-dihydro-2,5,7,8-tetramethyl-2H-1-benzopyran-6-ol.2HCl). Analogs and derivatives thereof can also be used.

The subject application provides compositions useful for inducing an increase in fatty acid oxidation and mitochondrial biogenesis in a subject. Such compositions contain: HMB in combination with resveratrol; leucine in combination with resveratrol; both leucine and HMB in combination with resveratrol; KIC in combination with resveratrol; both KIC and HMB in combination with resveratrol; both KIC and leucine in combination with resveratrol; or KIC, HMB and leucine in combination with resveratrol.

In some embodiments, a composition comprises an amount of a sirtuin pathway activator, such as a polyphenol (e.g. resveratrol). The amount of sirtuin pathway activator may be a subtherapeutic amount, and/or an amount that is synergistic with one or more other compounds in the composition or one or more other compounds administered simultaneously or in close temporal proximity with the composition. In some embodiments, the sirtuin pathway activator is administered in a low dose, a medium dose, or a high dose, which describes the relationship between two doses, and generally do not define any particular dose range. For example, a daily low dose of resveratrol may comprise about, less than about, or more than about 0.5 mg/kg, 1 mg/kg, 2.5 mg/kg, 5 mg/kg, 7.5 mg/kg, 10 mg/kg, 12.5 mg/kg, 15 mg/kg, 20 mg/kg, 25 mg/kg, 50 mg/kg, 75 mg/kg, 100 mg/kg, or more; a daily medium dose of resveratrol may comprise about, less than about, or more than about 20 mg/kg, 25 mg/kg, 50 mg/kg, 75 mg/kg, 100 mg/kg, 125 mg/kg, 150 mg/kg, 175 mg/kg, 200 mg/kg, 250 mg/kg, or more; and a daily high dose of resveratrol may comprise about, less than about, or more than about 150 mg/kg, 175 mg/kg, 200 mg/kg, 225 mg/kg, 250 mg/kg, 300 mg/kg, 350 mg/kg, 400 mg/kg, or more.

In some embodiments, the sirtuin pathway activator modulates the activity of phosphodiesterase (PDE). In some embodiments, the sirtuin pathway activator is a PDE inhibitor, such as a non-specific PDE inhibitor. PDE inhibitors can be naturally occurring or non-naturally occurring (e.g. manufactured), and may be provided in the form of a natural source comprising the PDE inhibitor, or an extract thereof (e.g. purified). Examples of non-specific PDE inhibitors include, but are not limited to, caffeine, theophylline, theobromine, 3-isobutyl-1-methylxanthine (IBMX), pentoxifylline (3,7-dihydro-3,7-dimethyl-1-(5oxohexyl)-1H-purine-2,6-dione), aminophylline, paraxanthine, and salts, derivatives, metabolites, catabolites, anabolites, precursors, and analogs thereof. Non-limiting examples of natural sources of PDE inhibitors include coffee, tea, guarana, verba mate, cocoa, and chocolate (e.g. dark chocolate).

In some embodiments, a PDE inhibitor is administered in place of or in addition to resveratrol or other sirtuin pathway activator. In some embodiments, compositions comprising one or more components described herein comprise a PDE inhibitor in place of or in addition to resveratrol or other sirtuin pathway activator. Typically, a PDE inhibitor is provided in an amount that is synergistic with one or more other components of a composition or method of treatment.

In some embodiments, the molar ratio of (a) leucine and/or metabolites thereof to (b) a sirtuin pathway activator is about or greater than about 20, 25, 30, 35, 40, 45, 50, 55, 60, 65, 70, 75, 80, 85, 90, 100, 120, or 150. In other embodiments, the molar ratio of leucine and/or metabolites thereof to sirtuin pathway activator contained in the subject compositions is about or greater than about 20, 30, 40, 50, 60, 70, 80, 90, 95, 90, 95, 100, 105, 110, 120, 130, 140, 150, 160, 170, 180, 190, 200, 220, 250, 300, 350, 400, or 500. In some embodiments, the molar ratio of component (a) to (b) in said composition is greater than about 20, 40, 60, 80, 100, 120, or 150. In some embodiments, the molar ratio of component (a) to (b) in said composition is greater than about 80, 100, 120, or 150. In some embodiments, the molar ratio of component (a) to (b) in said composition is greater than about 80, 100, 120, or 150. In some embodiments, the molar ratio of component (a) to (b) in said composition is greater than about 200, 250, or 300. In some embodiments, the molar ratio of component (a) to (b) in said composition is greater than about 40, 150, 250, or 500.

Additional Active Ingredients

The invention provides for the combination of (a) leucine and/or one or more leucine metabolites and (b) a sirtuin activator with one or more additional active ingredients. The additional active ingredients can be directed to weight loss, kidney disease maintenance or prevention, senior or old-age pets, dental diseases and conditions, and stool.

The invention also encompasses compositions for preventing, ameliorating one or more symptoms of, or treating certain disorders by administering a therapeutically or prophylactically effective amount of a composition to a companion animal in need thereof

In some embodiments, a pet food composition comprises about 0.05 to 5 wt % of leucine and/or about 0.005 to 1 wt % of one or more leucine metabolites; about 0.0005 to 0.05 wt % of a sirtuin activator; and an additional component selected from the group consisting of omega-3 fatty acid, eicosapentanoic acid, choline, manganese, methionine, cysteine, L-carnitine, lysine, alpha lipoic acid, dimethylaminoethanol, pyruvic acid, actyl L-carnitine, L-carnitine, conjugated linoleic acid, diacylglyceride, chondroitin, glucosamine, ginger (or extract thereof), chicory pulp, and myrtle. Optionally, the amount of leucine, leucine metabolite, and/or sirtuin activator can be any other amount described herein.

In other embodiments, a pet food, treat or supplement composition comprises at least about 50 mg of leucine and/or at least about 5 mg of one or more leucine metabolites; about 1 mg of a sirtuin activator; and an additional component selected from the group consisting of omega-3 fatty acid, eicosapentanoic acid, choline, manganese, methionine, cysteine, L-carnitine, lysine, alpha lipoic acid, dimethylaminoethanol, pyruvic acid, actyl L-carnitine, L-carnitine, conjugated linoleic acid, diacylglyceride, chondroitin, glucosamine, ginger (or extract thereof), chicory pump, and myrtle. Optionally, the amount of leucine, leucine metabolite, and/or sirtuin activator can be any other amount described herein.

Weight Loss

In some embodiments, the subject compositions can be include an additional weight loss component that can facilitate weight loss. The additional weight loss component can be pyruvic acid (pyruvate), L-carnitine, conjugated linoleic acid, diacylglyceride, and econa oil.

As used herein, the term “pyruvic acid or a salt thereof includes, but is not limited to, for example, pyruvic acid or carboxylate anion of pyruvic acid known as pyruvate. In various embodiments, the pyruvic acid or a salt thereof can be administered in a composition comprising a wet or dry food composition, which may be in the form of a moist food, dry food, supplement or treat. The pyruvic acid or a salt thereof may be incorporated therein or on the surface of any food composition, such as, by spraying or precipitation thereon or may be added to the diet by way of snack, supplement, treat or in the liquid portion of the diet such as water or another fluid. The pyruvic acid or a salt thereof may be administered as a powder, solid or as a liquid including a gel. An important aspect is that the animal be provided an effective amount of the pyruvic acid or a salt thereof to provide a positive effect.

A combination of lipoic acid or salt thereof and pyruvic acid or salt thereof for consumption by a companion animal can provide treatment for obesity. Adding a combination of lipoic acid or salt thereof and pyruvic acid or salt thereof to a composition for consumption can also decreases body fat and increases lean muscle mass.

Carnitine is found in the body and is enzymatically combined with fatty acids to facilitate their transportation through mitochondrial membranes, thus aiding in fatty acid metabolism (Yalkowsky, S. H., 1970). Oral administration of L-carnitine for obesity in mammals has been described in U.S. Pat. No. 3,810,994.

A diacylglyceride is a lipid structurally characterized by a glycerol (a three carbon alcohol) backbone, two fatty acid chains and a phosphate group. By definition, a 1,2-diacylglyceride comprises fatty acid chains located at carbons 1 and 2 and, further, are characterized by a long hydrocarbon molecule such as unsaturated, saturated and conjugated hydrocarbons. 1,2-diacylglyceride is a precursor to phosphatidylcholine, phosphatidylethanolamine and phosphatidylinositol, which are indispensable components of biological membranes. In addition, 1,2-diacylglycerides are precursors to triglyceride biosynthesis and, therefore, is central to energy stores of organisms. However, 1,3-diacylglycerides are not metabolized to triglyceride and, thus, are not deposited as fat but rather, burned as energy. 1,3-diacylglyceride differs structurally from 1,2-diacylglycerides by having an acyl group at C-3 of the glycerol backbone rather than at C-2. Clinical investigations of obesity have included dietary consumption of diacylglycerol and indicated that diacylglycerol lowers serum triglyceride and cholesterol levels (Takasaka et al., 2000) and decreases body weight and regional fat deposition (Nagao et al., 2000). Rich natural sources of 1,3-diacylglycerides are vegetable oils, such as Econa oil.

Functional ingredients that modulate metabolism and build lean body mass can include L-carnitine, conjugated linoleic acid and a diacylglyceride, and can include conjugated linoleic acid and diacylglyceride, and can include a diacylglyceride.

A subject composition, which may be a pet food, treat, snack, supplement, or drink, can comprise pyruvate or pyruvic acid content of at least about 0.01, 0.05, 0.1, 0.5, 1, 2, 3, 4, 5, 6, 7, 8, 9, 10, 14, 18, 20, 24% by weight on a dry matter basis. In some instances, the composition comprises a pyruvate or pyruvic acid content of about 0.1 to 10% by weight on a dry matter basis of subject composition.

A subject composition, which may be a pet food, treat, snack, supplement, or drink, can comprise at least about 5, 10, 20, 30, 40, 50, 60, 70, 80, 90, 100 ppm L-carnitine. A subject composition can comprise at least about 0.01%, 0.05%, 0.1%, 0.5%, 1%, 1.5%, 2%, 2.5%, 3% L-carnitine. In some instances, a subject composition comprises about 0.1 to 1% L-carnitine of the subject composition. As used herein, units of ppm can be equivalent to mg/kg.

A subject composition, which may be a pet food, treat, snack, supplement, or drink, can comprise at least about 0.01%, 0.05%, 0.1%, 0.5%, 1%, 1.5%, 2%, 2.5%, 3% linoleic acid. In some instances, a subject composition comprises about 0.1 to 1% L-carnitine of the subject composition.

A subject composition, which may be a pet food, treat, snack, supplement, or drink, can comprise at least about 0.01%, 0.05%, 0.1%, 0.5%, 1%, 1.5%, 2%, 2.5%, 3% diacylglyceride. In some instances, a subject composition comprises about 0.1 to 1% diacylglyceride of the subject composition.

A subject composition, which may be a pet food, treat, snack, supplement, or drink, can comprise at least about 0.01%, 0.05%, 0.1%, 0.5%, 1%, 1.5%, 2%, 2.5%, 3% econa oil. In some instances, a subject composition comprises about 0.1 to 1% econa oil of the subject composition.

A subject composition, which may be a pet food, treat, snack, supplement, or drink, can comprise pyruvate or pyruvic acid content of at least about 10, 50, 100, 200, 300, 400, 500, 600, 700, 800, 900 mg of the subject composition. In some instances, the composition comprises a pyruvate or pyruvic acid content of about 300-700 mg of subject composition

A subject composition, which may be a pet food, treat, snack, supplement, or drink, can comprise at least about 5, 10, 20, 30, 40, 50, 60, 70, 80, 90, 100 mg L-carnitine. In some instances, L-carnitine comprises about 20 to about 100 mg of the subject composition.

A subject composition, which may be a pet food, treat, snack, supplement, or drink, can comprise an linoleic acid content of at least about 25, 50, 75, 100, 125, 150, 175, 200, 250, 300, 350, 400 mg of the subject composition. In some instances the composition comprises an linoleic acid content of at least about 150 to about 300 mg of the subject composition.

A subject composition, which may be a pet food, treat, snack, supplement, or drink, can comprise diacylglyceride content of at least about 25, 50, 75, 100, 125, 150, 175, 200, 250, 300, 350, 400 mg of the subject composition. In some instances the composition comprises diacylglyceride content of at least about 150 to about 300 mg of the subject composition.

A subject composition, which may be a pet food, treat, snack, supplement, or drink, can comprise econa oil content of at least about 25, 50, 75, 100, 125, 150, 175, 200, 250, 300, 350, 400 mg of the subject composition. In some instances the composition comprises econa oil content of at least about 150 to about 300 mg of the subject composition.

Kidney Disease

In other embodiments, the subject compositions can include active ingredients directed to kidney disease, for example pyruvate.

Glomerulonephritis or glomerular nephritis (“GN”) is a renal disease which is characterized by inflammation of the glomeruli or capillary loops of the kidney. It is a pathologic process associated with a number of diverse underlying diseases. The condition occurs in acute, sub-acute and chronic forms and also secondary to an infection. The former conditions, where a concurrent illness cannot be found, are generally referred to as idiopathic glomerulonephritis. The latter conditions are generally referred to as secondary glomerulonephritis. Whatever the underlying cause, immune complexes form and result in a series of events leading to glomerular injury and loss of renal function, proteinuria and ultimately, in some cases, renal failure.

Diet plays an important role in kidney disease causation and progression because it is fundamentally involved in metabolism. Biological pathways are at some level regulated by nutritional factors. Thus, dietary components present in foods as nutrients may regulate gene expression at the transcriptional and translational level, as well as in certain post-translational modifications. They may similarly be involved in degradation and enzymatic activities. Nutrient levels may influence the equilibrium of metabolic pathways. Metabolic pathways are frequently complex and may involve many redundancies and interrelationships among different metabolic pathways. Altering the concentration of a single enzyme, growth factor, cytokine or metabolite may impact a number of metabolic pathways involved in disease-related physiology. Hormones and other cell signaling molecules are well-understood to be regulated by diet and are also known to be implicated in the development and progression of disease.

As used herein, the term “pyruvic acid or a salt thereof includes, but is not limited to, for example, pyruvic acid or carboxylate anion of pyruvic acid known as pyruvate. In various embodiments, the pyruvic acid or a salt thereof can be administered in a composition comprising a wet or dry food composition, which may be in the form of a moist food, dry food, supplement or treat. The pyruvic acid or a salt thereof may be incorporated therein or on the surface of any food composition, such as, by spraying or precipitation thereon or may be added to the diet by way of snack, supplement, treat or in the liquid portion of the diet such as water or another fluid. The pyruvic acid or a salt thereof may be administered as a powder, solid or as a liquid including a gel. An important aspect is that the animal be provided an effective amount of the pyruvic acid or a salt thereof to provide a positive effect. Typically, the source of pyruvic acid or a salt thereof is present in the composition in an amount of up to an amount, which remains non-toxic to the animal.

A subject composition, which may be a pet food, treat, snack, supplement, or drink, can comprise pyruvate or pyruvic acid content of at least about 0.01, 0.05, 0.1, 0.5, 1, 2, 3, 4, 5, 6, 7, 8, 9, 10, 14, 18, 20, 24% by weight on a dry matter basis. In some instances, the composition comprises a pyruvate or pyruvic acid content of about 0.1 to 10% by weight on a dry matter basis of subject composition.

A subject composition, which may be a pet food, treat, snack, supplement, or drink, can comprise pyruvate or pyruvic acid content of at least about 100, 200, 300, 400, 500, 600, 700, 800, 900 mg of the subject composition. In some instances, the composition comprises a pyruvate or pyruvic acid content of about 300-700 mg of subject composition.

Senior Pets

In some embodiments, the subject compositions can include active ingredients that address issues associated with senior or old-age pets or animals. Active ingredients directed to senior or old-age pets can include chondroitin, glucosamine, omega-3-polyunsaturated fatty acids, choline, manganese, methionine, cysteine, L-carnitine, lysine, eicosapentanoic acid, vitamin E, vitamin C, and alpha-lipoic acid.

Essential fatty acids, consisting of omega-3 and omega-6 polyunsaturated fatty acids, are critical nutrients for the health of an animal. These nutrients, however, either cannot be made by animals or cannot be made in sufficient amounts to elicit benefits and therefore must be consumed in an animal's diet. See, e.g., Hornstra, G., et al., “Essential fatty acids in pregnancy and early human development”, Eur. J. Obs. & Gyn. and Reprod. Biology, 61:57-62 (1995). It has previously been postulated that Docosahexaenoic Acid (“DHA”), an omega-3 polyunsaturated fatty acid, is effective in increasing the maze-learning ability and brain functions in aged mice. See, Lim, S.-Y., “Intakes of dietary docosahexaenoic acid ethyl ester and egg phosphatidylcholine improve maze-learning ability in young and old mice”, J. Nutr., 130:1629-1632 (2000).

The compositions for use in the methods of the present invention generally have an omega-3 polyunsaturated fatty acid content of at least about 25, 50, 75, 100, 125, 150, 175, 200, 225, 250, 300, 400, 500 mg. The omega-3 polyunsaturated fatty acid can be DHA. In other embodiments, the omega-3 polyunsaturated fatty acid is EPA. In still other embodiments, the omega-3 polyunsaturated fatty acid comprises a mixture of DHA and EPA

A subject composition, which may be a pet food, treat, snack, supplement, or drink, can comprise an omega-3 polyunsaturated fatty acid content of at least about 0.005%, 0.01%, 0.05%, 0.1%, 0.5%, 1%, 2%, 3%, 3.5%, 4%, 5%, 6%, 7% by weight on a dry matter basis. The omega-3 polyunsaturated fatty acid can be DHA. In other embodiments, the omega-3 polyunsaturated fatty acid is EPA. In still other embodiments, the omega-3 polyunsaturated fatty acid comprises a mixture of DHA and EPA.

The omega-3 fatty acids may be obtained from a variety of sources. One convenient source is fish oils from, for example, menhaden, mackerel, herring, anchovy, and salmon. DHA and EPA are typical fatty acids present in such fish oils, and, together often make up a significant portion of the oil, such as from about 25% to about 38% of the oil.

In some embodiments, the composition containing omega-3 polyunsaturated fatty acid is a food. Although both liquid and solid foods are provided, solid foods are typically preferred. Foods include both dry foods and wet foods.

Rogers discusses the theory of the potential use of antioxidants to slow the deterioration of cognitive function, particularly in the elderly. See Rogers, P., “A healthy body, a healthy mind: long-term impact of diet on mood and cognitive function”, Proceedings of the Nutrition Society, 60:135-143 (2001).

The term “antioxidant” means a substance that is capable of reacting with free radicals and neutralizing them. Illustrative examples of such substances include beta-carotene, selenium, coenzyme Q10 (ubiquinone), luetin, tocotrienols, soy isoflavones, S-adenosylmethionine, glutathione, taurine, N-acetylcysteine, vitamin E, vitamin C, lipoic acid and L-carnitine. Examples of foods containing useful levels of one or more antioxidants include but are not limited to ginkgo biloba, green tea, broccoli, citrus pulp, grape pomace, tomato pomace, carrot spinach, and a wide variety of fruit meals and vegetable meals. Without being bound by theory, the health benefits may be the result of physiological effects from the addition of omega-3 polyunsaturated fatty acids to a senior or super senior animal's diet. Similarly, the antioxidants, choline, and other nutrients may play a role in enhancing a senior or super senior animal's quality of life.

A subject composition, which may be a pet food, treat, snack, supplement, or drink, can comprise an amount of Vitamin C or Vitamin E that is at least about or about 10, 20, 30, 50, 75, 100, 125, 150, 200, 300, 500, 1000 or 5000 ppm of the pet food composition. A subject composition, which may be a pet food, treat, snack, supplement, or drink, can comprise an amount of Vitamin C or Vitamin E that is at least about or about 50, 100, 200, 250 300, 600, 1000, 1500, 2000, 3000, or 5000 mg.

A subject composition, which may be a pet food, treat, snack, supplement, or drink, can comprise an L-carnitine content of at least about 5, 10, 20, 30, 40, 50, 60, 70, 80, 90, 100 ppm of the subject composition. In some instances the composition comprises an L-carnitine content of at least about 50 ppm of the subject composition.

A subject composition, which may be a pet food, treat, snack, supplement, or drink, can comprise an L-carnitine content of at least about 25, 50, 75, 100, 125, 150, 175, 200, 250, 300, 350, 400 mg of the subject composition. In some instances the composition comprises an L-carnitine content of at least about 250 mg of the subject composition.

A subject composition, which may be a pet food, treat, snack, supplement, or drink, can comprise an amount of eicosapentanoic acid that is at least about or about 0.1, 0.2, 0.4, 0.8, or 1 wt % of the pet food composition. A subject composition, which may be a pet food, treat, snack, supplement, or drink, can comprise an amount of eicosapentanoic acid that is at least about or about 50, 100, 200, 300, or 600 mg.

A subject composition, which may be a pet food, treat, snack, supplement, or drink, can comprise a choline content of at least about 100, 200, 300, 500, 600, 700, 800, 900, 1000, 1200, 1400, 1600, 1800, 2000 ppm. In some instances the composition comprises a choline content of at least about 1000 ppm of subject composition.

A subject composition, which may be a pet food, treat, snack, supplement, or drink, can comprise a choline content of at least about 100, 200, 300, 500, 600, 700, 800, 900, 1000, 1200, 1400, 1600, 1800, 2000 mg of the subject composition. In some instances the composition comprises a choline content of at least about 1000 mg of subject composition.

The subject compositions can also include chondroitin and glucosamine. The bioavailability of glucosamine and GAGs, such as chondroitin sulfate, is believed to bolster the restoration of connective tissue in a variety of ways. For example, glucosamine stimulates the incorporation of other precursors, such as GAGs, PGs, and collagen, into the connective tissue matrix and serves as a precursor to GAG synthesis. Chondroitin sulfate serves to inhibit degradative enzymes and stimulates GAG and PG synthesis in connective tissue cells, particularly chondrocytes. The vertebrate body synthesizes GAGs, such as in chondrocytes (cartilage), fibroblasts (skin, ligaments), and osteoblasts (bone),

Without being limited to theory, the connective tissue of vertebrates includes bone and cartilage as well as tissue that underlies the skin, envelops muscle, and occupies space between internal organs. The primary building blocks of connective tissue are proteoglycans (PG), which are linked to collagen fibers to form connective tissue. Proteoglycan subunits are comprised of glycoaminoglycans (also known as GAGs or mucopolysaccharides) attached in large numbers to a core protein, with these proteoglycan subunits being attached to a very long hyaluronic acid molecule via protein links to form aggregating proteoglycan. GAGs are long-chain polymers with monomer units comprising an aminosugar and an organic acid or sugar. One type of GAG is chondroitin sulfate, which is composed of glucuronic acid and N-acetyl galactosamine sulfate. Glucosamine is a key precursor to both GAGs and hyaluronic acid molecules, which are the primary components of PGs, as discussed above. In fact, the bioavailability of glucosamine is the rate-limiting step in the synthesis of GAGs and PGs.

A subject composition, which may be a pet food, treat, snack, supplement, or drink, can comprise a chondroitin content of at least about 50, 100, 150, 200, 250, 300, 350, 400, 450, 500, 550, 600, 650, 700, 750, 800, 850, 900, 950, 1000 mg of the subject composition. In some instances the composition comprises a chondroitin content of at least about 500 mg of the subject composition.

A subject composition, which may be a pet food, treat, snack, supplement, or drink, can comprise a glucosamine content of at least about 30, 50, 100, 150, 200, 250, 300, 350, 400, 450, 500, 550, 600 mg of the subject composition. In some instances the composition comprises a glucosamine content of at least about 300 mg of the subject composition.

A subject composition, which may be a pet food, treat, snack, supplement, or drink, can comprise a chondroitin content of at least about 0.05%, 0.1%, 0.2%, 0.3%, 0.5%, 0.6%, 0.7%, 0.8%, 1%, 1.5% of the subject composition. In some instances the composition comprises a lysine content of at least about 0.5% of the subject composition.

A subject composition, which may be a pet food, treat, snack, supplement, or drink, can comprise a glucosamine content of at least about 0.03%, 0.05%, 0.1%, 0.2%, 0.3%, 0.5%, 0.6%, 0.7%, 0.8%, 1%, 1.5% of the subject composition. In some instances the composition comprises a lysine content of at least about 0.3% of the subject composition.

The compositions for use in the methods of this invention further comprise at least one nutrient selected from the group consisting of manganese, methionine, cysteine, mixtures of methionine and cysteine, L-carnitine, lysine, and arginine. Specific preferred amounts for each component in a composition will depend on a variety of factors including, for example, the species of animal consuming the composition; the particular components included in the composition; the age, weight, general health, sex, and diet of the animal; the animal's consumption rate, and the like. Thus, the component amounts may vary widely, and may even deviate from the proportions given herein.

A subject composition, which may be a pet food, treat, snack, supplement, or drink, can comprise a manganese content of at least about 5, 10, 20, 40, 50, 60, 80, 100, 120, 140, 150, 160, 180, 200, 250, 300 ppm. In some instances the composition comprises a manganese content of at least about 50 ppm, at least about 50 ppm to at about 150 ppm, or from about 100 ppm to about 150 ppm, or from about 100 ppm to about 110 ppm of the subject composition.

A subject composition, which may be a pet food, treat, snack, supplement, or drink, can comprise a manganese content of at least about 5, 10, 20, 40, 50, 60, 80, 100, 120, 140, 150, 160, 180, 200, 250, 300 mg of the subject composition. In some instances the composition comprises a manganese content of at least about 50 mg, at least about 50 mg to at about 150 mg, or from about 100 mg to about 150 mg, or from about 100 mg to about 110 mg of the subject composition.

A subject composition, which may be a pet food, treat, snack, supplement, or drink, can comprise a methionine content of at least about 0.04%, 0.08%, 0.1%, 0.5%, 1%, 1.5%, 2%, 2.5%, 3% of the subject composition. In some instances the composition comprises a methionine content of at least about 0.4% to 1.5% of the subject composition.

A subject composition, which may be a pet food, treat, snack, supplement, or drink, can comprise a methionine content of at least about 0.75, 1, 1.25, 1.5, 1.75, 2, 2.25, 2.5, 3, 3.5 mg of the subject composition. In some instances the composition comprises a methionine content of at least about 7.5 mg of the subject composition.

A subject composition, which may be a pet food, treat, snack, supplement, or drink, can comprise a lysine content of at least about 0.04%, 0.08%, 0.1%, 0.5%, 1%, 1.5%, 2%, 2.5%, 3%, 4% ppm of the subject composition. In some instances the composition comprises a lysine content of at least about 0.4%, between about 0.4 to 2%, between about 0.9 to 2%, or between about 0.9 to 1.2% of the subject composition.

A subject composition, which may be a pet food, treat, snack, supplement, or drink, can comprise a lysine content of at least about 40, 100, 150, 200, 250, 300, 350, 400, 450, 500, 550, 600, 650, 700, 750, 800, 850 mg of the subject composition. In some instances the composition comprises a lysine content of at least about 400 mg of the subject composition

Dental

In other embodiments, the subject compositions can include active ingredients that address dental issues, for example myrtle.

The inhibition of certain plaque biofilm forming bacteria by myrtle results in the control or reduction of dental plaque in an animal by the reduction of the bacterial content of the dental plaque. The food product is consumed and during use the oral cavity of the animal is exposed to the myrtle of the food product, and the composition can have direct contact with the surface of a tooth of the animal. The surface of a tooth can be directly contacted with the myrtle of the food product, as well as being contacted by the pet food composition to physically remove a proportion of the plaque. The food product of the invention has the benefit of improving or maintaining the oral health of the animal by removing plaque through the mechanical (gentle abrasive) action of the product against the surface of the teeth of the animal, as well as by the action of the myrtle.

A subject composition, which may be a pet food, treat, snack, supplement, or drink, can comprise a myrtle content of at least about 0.1%, 0.5%, 1%, 2%, 3%, 4%, 5%, 6%, 7%, 8%, 9%, 10%, 12%, 14%, 16%, 18%, 20% of the subject composition. In some instances the composition comprises myrtle content of about 1 to about 10% of the subject composition.

A subject composition, which may be a pet food, treat, snack, supplement, or drink, can comprise a myrtle content of at least about 75 100, 150, 200, 250, 300, 350, 400, 450, 500, 550, 600, 650, 700, 750, 800, 900, 1000, 1500, 2000 mg of the subject composition. In some instances the composition comprises myrtle content of about 750 mg of the subject composition.

Stool

In some embodiments, the subject compositions can include active ingredients that improve stool, such as by improving the odor or consistency of the stool. Odor-improving active ingredients include ginger. Consistency improving active ingredients can include chicory. Additionally, the compositions can be formulated to have a selected balance of cations and anions such that the stool of the animal is improved.

Pet health can be monitored in a number of ways. Two of these are feces quality and gastrointestinal (GI) tract health. Good quality feces in pet animals is of two-fold importance. Firstly, it is a good indicator of a healthy pet. It is known that good feces quality usually reflects healthy colonic structure and function.

Stool quality and stool frequency are generally determined by five factors: food ingredient digestibility, fiber level, health status, activity level, and water intake. When these factors are balanced, stools are generally formed, firm, dark, and exhibit a relatively reduced odor. Stools exhibiting these properties are considered to be good quality stools. If the factors are not balanced, stools are generally soft, loose, watery, light-colored, and exhibit a relatively increased odor. Stools exhibiting these properties, particularly loose, watery stools, are considered to be poor quality stools.

Poor stool quality and irregular stool frequency can be caused by various factors, e.g., abnormal intestinal motility, increases in intestinal permeability, the presence of nonabsorbable osmotically active substances in the intestine, or agents that cause diarrhea. Similarly, some animal foods, particularly those known in the art as chunk and gravy animal foods, can cause poor stool quality. Often, an animal consuming such foods has a fecal discharge that is irregular and undesirable. Such discharge is generally characterized by frequent loose, watery stools. In some instances, the discharge may be classified as diarrhea.

One method for maintaining normal gastrointestinal function and ameliorating chronic diarrhea in animals can include the addition, in pet food products, of a fiber source, such as chicory, which contains a significantly higher proportion of insoluble fibers to soluble fibers, which are either non-fermentable or only moderately fermentable.

In some embodiments, a pet food composition can comprise chicory pulp in an amount which: i) maintains good feces quality or improves the feces quality of a pet and/or ii) maintains good gastrointestinal tract health and/or improves the gastrointestinal tract health of a pet; about 0.05 to 5 wt % of leucine and/or about 0.005 to 1 wt % of one or more leucine metabolites; and about 0.0005 to 0.05 wt % of a sirtuin activator. Optionally, the amount of leucine, leucine metabolite, and/or sirtuin activator can be any other amount described herein.

Chicory is a blue flowered plant (Cichorium intybus), named ENDIVE in the US, which is cultivated for its salad leaves and its root (roasted and ground for use with, or instead of, coffee). Its primary components are i) inulin and ii) soluble and fermentable fibers. Inulin is a soluble polysaccharide, composed of polymerized fructose molecules, occurring as stored food material in many plants, such as members of the Compositae species and in dahlia tubers. However, it is not the endogenous inulin of chicory, but rather the fiber, which imparts the advantageous effect in maintaining and/or improving fecal quality in pet animals.

The most common extraction process is of chicory root and is similar to the extraction of sucrose from sugar beet (diffusion in water). The extraction removes inulin to leave a chicory pulp.

A subject composition, which may be a pet food, treat, snack, supplement, or drink, can comprise a chicory content of at least about 0.5%, 1%, 2%, 3%, 4%, 5%, 10%, 15%, 20%, 25%, 30%, 35%, 40%, 45%, 50% of the subject composition. In some instances the composition comprises chicory content of about 5 to about 20% of the subject composition. In some instances the composition comprises chicory content of about 2 to about 10% of the subject composition.

A subject composition, which may be a pet food, treat, snack, supplement, or drink, can comprise a chicory content of at least about 35, 50, 100, 150, 200, 250, 300, 350, 400, 450, 500, 550, 600, 650, 700 mg of the subject composition. In some instances the composition comprises chicory content of at least about 350 ppm of the subject composition.

Excreta odor in companion animals is an unpleasant reality of living with pets. For owners of animals that live indoors, especially cats and dogs that use litter boxes or are confined to kennels or other small spaces, this problem is particularly unpleasant. Cat litter containing deodorizers has been developed, however this is an imperfect solution to the problem. Excreta odor in animals is partially a result of indigestion and microbial fermentation caused by inappropriate bacterial activity, inflammation and poor digestion or motility.

Ginger and extracts thereof can be effective in reducing odor of excreta from an animal when included in the animal's diet. Without being held to a particular theory, it is believed that the beneficial effects of ginger result at least in part from antimicrobial, anti-inflammatory and gastric stimulation properties of ginger. Specifically, it is believed that ginger reduces the level of odor producing compounds including heterocycles, phenols, thiols, sulfides and indoles present in excreta, and in this way reduces excreta odor including fecal, urinary and flatulence odor.

The invention provides for a pet food composition for reducing odor of stool of a companion animal comprising: stool odor-reducing effective amount of ginger or an extract thereof; about 0.05 to 5 wt % of leucine and/or about 0.005 to 1 wt % of one or more leucine metabolites; and about 0.0005 to 0.05 wt % of a sirtuin activator. The invention also provides for a method for reducing odor of stool of a companion animal, the method comprising causing the animal to ingest a pet food composition comprising: a stool odor-reducing effective amount of ginger or an extract thereof; about 0.05 to 5 wt % of leucine and/or about 0.005 to 1 wt % of one or more leucine metabolites; and about 0.0005 to 0.05 wt % of a sirtuin activator. Optionally, the amounts of leucine, leucine metabolite, and/or sirtuin activator can be any other amount described herein.

For purposes of this application, “ginger” includes plant parts, particularly rhizomes (sometimes referred to as “roots”), of Zingiber officinale and other Zingiber species. This includes ginger in any form such as powder, dehydrated, fresh, cooked or raw. An “extract” of ginger herein is any preparation containing substances extracted from ginger, including fluid extracts, tinctures, essential oils, distillates and oleoresins

A subject composition, which may be a pet food, treat, snack, supplement, or drink, can comprise a ginger content of at least about 0.0005%, 0.001%, 0.005%, 0.01%, 0.05%, 0.1%, 0.5%, 1%, 2%, 4%, 6%, 8%, 10%, 12%, 14%, 16%, 18%, 20%, 22%, 24% of the subject composition. In some instances the composition comprises ginger content of about 0.005 to about 12% of the subject composition.

A subject composition, which may be a pet food, treat, snack, supplement, or drink, can comprise a ginger content of at least about 50, 100, 150, 200, 250, 300, 350, 400, 450, 500, 550, 600, 650, 700, 750, 800, 850, 900, 950, 1000 mg of the subject composition. In some instances the composition comprises ginger content of about 500 mg of the subject composition.

U.S. patent application Ser. No. 11/566,512 (the '512 application) filed Dec. 4, 2006 (the contents of which are herein incorporated by reference) discloses adjusting the balance of metabolizable cations to metabolizable anions consumed by the animal to affect stool quality. It has generally been found that increasing the balance of metabolizable cations to metabolizable anions will result in firmer stool quality and reduced stool output. Conversely, decreasing the balance of metabolizable cations to metabolizable anions will result in looser stool and increased stool output.

While the '512 application describes methods for altering stool quality and/or frequency for adult dogs, the dietary requirements of adult dogs and puppies are quite different. For example, puppies generally experience rapid growth and development; thus they generally require significantly more nutrition than adult dogs and of a different nutritional composition. For example, puppies require higher calcium levels due to their rapid bone growth. Similarly, the stool of adult dogs and puppies are typically of different quality and it is known that a dog produces substantially firmer stool than a puppy, partly in response to their different diets and nutritional requirements. Thus, the cation and anion balance disclosed in the prior art, while suitable for adult dogs, may be unsuitable for improving the stool quality of a puppy. The stool quality of a puppy may be improved by modifying the dietary cation and anion balance of a food composition consumed by the animal such that the dietary cation and anion balance is from about 50 to about 300 mEq.

As used herein, “altering stool quality”, “modifying stool quality” or “improving stool quality” refers to modifying the stool of an animal to produce a desired firmness in the stool and/or a desired stool frequency. Generally, stools that are loose and watery are not desired, nor are stools that are so firm that constipation is observed. Thus, stool quality is improved in an animal experiencing diarrhea (e.g., frequent loose, watery stools) by causing the stool to be more firm and causing the animal to produce fewer stools; conversely, an animal experiencing constipation will benefit by a change in stool quality such that the stool is less firm. As described herein, such changes may be achieved by altering the animal's dietary cation-anion balance (DCAB). An increase in DCAB can cause stool to be firmer; a decrease in DCAB can cause stool to be less firm.

Stool quality may be scored according to methods familiar to one of skill in the art. For example, fecal quality is commonly assessed by those of skill in the art by visual scoring, e.g., ranking stool visually on a scale from grade 1-5 as follows: Grade 1: Greater than two-thirds of the feces in a defecation are liquid. The feces have lost all form, appearing as a puddle or squirt.

Grade 2: Soft-liquid feces are an intermediate between soft and liquid feces. Approximately equal amounts of feces in a defecation are soft and liquid. Grade 3: Greater than two-thirds of the feces in a defecation are soft. The feces retain enough form to pile but have lost their firm cylindrical appearance. Grade 4: Firm-soft feces are an intermediate between the grades of firm and soft. Approximately equal amounts of feces in a defecation are firm and soft. Grade 5: Greater than two-thirds of the feces in a defecation are firm. They have a cylindrical shape with little flattening. See also, Sunvold et al., J Anim Sci 1995 73:1099-1109; U.S. Pat. No. 6,280,779; U.S. Pat. No. 5,616,569. Stool quality may also be assessed quantitatively using methods to determine the amount of moisture in the feces in g/kg (see, e.g., Yamka et al., 2006, Am J Vet Res, 67(1):88-94).

The balance of metabolizable cations to metabolizable anions in the present invention can be determined by any means known to skilled artisans. For example, one method for measuring the balance of metabolizable cations to metabolizable anions is to calculate the animal's dietary cation-anion balance (DCAB), which is determined by calculating the cumulative amount of cations regularly consumed by the animal and subtracting the cumulative amount of anions consumed by the animal. Cations may include, e.g., sodium, potassium, calcium, and magnesium cations, or any other ion having a positive charge, including amino acids. Anions may include, e.g., chloride, sulfur, and phosphorus anions, or any other ion having a negative charge, including amino acids. For example, the DCAB is determined by calculating the cumulative amounts of sodium, potassium, calcium, and magnesium cations regularly consumed by the animal and subtracting the cumulative amount of chloride, sulfur, and phosphorus anions regularly consumed by the animal.

In some embodiments, a pet food compositions can comprise about 0.05 to 5 wt % of leucine and/or about 0.005 to 1 wt % of one or more leucine metabolites; about 0.0005 to 0.05 wt % of a sirtuin activator; and a pre-selected balance of metabolizable cations to metabolizable anions, wherein the DCAB is between about 50 to 300. Optionally, the amount of leucine, leucine metabolite, and/or sirtuin activator can be any other amount described herein.

The invention also provides for a method for treating a puppy susceptible to or suffering from diarrhea and/or loose stool comprising: feeding the puppy a food composition comprising (i) about 0.05 to 5 wt % of leucine and/or about 0.005 to 1 wt % of one or more leucine metabolites and (ii) about 0.0005 to 0.05 wt % of a sirtuin activator; and adjusting the balance of metabolizable cations to metabolizable anions consumed by the puppy by an amount sufficient to improve stool quality by increasing the balance of metabolizable cations to metabolizable anions consumed by the puppy to produce firmer stool. Optionally, the amount of leucine, leucine metabolite, and/or sirtuin activator can be any other amount described herein.

The DCAB can be determined in accordance with the following equation: DCAB (mEq)=(Na+K+Ca+Mg)−(Cl+S+P). For purposes of calculating the DCAB, sodium, potassium and chloride ions have a valance of 1, sulfur, calcium and magnesium ions have a valance of 2, and phosphorus ions have a valance of 1.8.

Probiotics and Prebiotics

In other embodiments, the subject compositions can include one or more probiotics or prebiotics. In addition to the optional use of anti-diarrhea agents and anti-constipation agents in the systems, compositions, methods, and kits of the present invention, in some embodiments, the balance of metabolizable cations to metabolizable anions consumed by the animal can be adjusted in conjunction with the administration of one or more compositions comprising a gastrointestinal tract-improving agent. “Gastrointestinal tract-improving agents” are generally probiotics and prebiotics.

The pet food composition can comprise a live probiotic microorganism; about 0.05 to 5 wt % of leucine and/or about 0.005 to 1 wt % of one or more leucine metabolites; and about 0.0005 to 0.05 wt % of a sirtuin activator. Optionally, the amount of leucine, leucine metabolite, and/or sirtuin activator can be any other amount described herein.

In other embodiments, the pet food composition comprises a live probiotic microorganism; at least about 50 mg of leucine and/or at least about 5 mg of one or more leucine metabolites by weight of the pet food composition; and at least about 1 mg of a sirtuin activator by weight of the pet food composition. Optionally, the amount of leucine, leucine metabolite, and/or sirtuin activator can be any other amount described herein.

Probiotics are live microorganisms that have a beneficial effect in the prevention and treatment of specific medical conditions when ingested. Probiotics are believed to exert biological effects through a phenomenon known as colonization resistance. The probiotics facilitate a process whereby the indigenous anaerobic flora limits the concentration of potentially harmful (mostly aerobic) bacteria in the digestive tract. Other modes of action, such as supplying enzymes or influencing enzyme activity in the gastrointestinal tract, may also account for some of the other functions that have been attributed to probiotics. Probiotics may enhance an animal's systemic cellular immune responses and may be useful as a dietary supplement to boost natural immunity in otherwise healthy animals. Probiotics include many types of bacteria but generally are chosen from four genera of bacteria: Lactobacillus acidophilus, Bifidobacteria, Lactococcus, and Pediococcus.

Prebiotics are nondigestible food ingredients that beneficially affect host health by selectively stimulating the growth and/or activity of bacteria in the colon, and are known by those of skill in the art. For example, fructooligosaccharide (FOS) is found naturally in many foods such as wheat, onions, bananas, honey, garlic, and leeks. FOS can also be isolated from chicory root or synthesized enzymatically from sucrose. FOS fermentation in the colon results in a large number of physiologic effects including increasing the numbers of bifidobacteria in the colon, increasing calcium absorption, increasing fecal weight, shortening of gastrointestinal transit time, and possibly lowering blood lipid levels. The increase in bifidobacteria has been assumed to benefit health by producing compounds to inhibit potential pathogens, by reducing blood ammonia levels, and by producing vitamins and digestive enzymes. Probiotic bacteria such as Lactobacilli or Bifidobacteria are believed to positively affect the immune response by improving the intestinal microbial balance leading to enhanced antibody production and phagocytic activity of white blood cells.

The amount of probiotics and prebiotics to be administered to the animal is determined by the skilled artisan based upon the type and nature of the probiotic and prebiotic and the type and nature of the animal, e.g., the age, weight, general health, sex, extent of microbial depletion, presence of harmful bacteria, and diet of the animal. Generally, probiotics are administered to the animal in amounts of from about one to about twenty billion colony forming units (CFUs) per day for the healthy maintenance of intestinal microflora, from about 5 billion to about 10 billion live bacteria per day. Generally, prebiotics are administered in amounts sufficient to positively stimulate the healthy microflora in the gut and cause these “good” bacteria to reproduce. Typical amounts are from about one to about 10 grams per serving or from about 5 percent to about 40 percent of the recommended daily dietary fiber for an animal.

In certain embodiments, the probiotic microorganism is Saccharomyces cereviseae, Bacillus coagulans, Bacillus licheniformis, Bacillus subtilis, Bifidobacterium bifidum, Bifidobacterium infantis, Bifidobacterium longum, Enterococcus faecium, Enterococcusfaecalis, Lactobacillus bulgaricus, Lactobacillus acidophilus, Lactobacillus alimentarius, Lactobacillus casei subsp. casei, Lactobacillus casei Shirota, Lactobacillus curvatus; Lactobacillus delbruckii subsp. lactis. Lactobacillus farciminus, Lactobacillus gasseri, Lactobacillus helveticus, Lactobacillus johnsonii, Lactobacillus reuteri, Lactobacillus rhamnosus (Lactobacillus GG), Lactobacillus sake, Lactobacillus sporogenes, Lactococcus lactis, Micrococcus varians, Pediococcus acidilactici, Pediococcus pentosaceus, Pediococcus acidilactici, Pediococcus halophilus, Streptococcus faecalis, Streptococcus thermophilus, Streptococcus faecium, Staphylococcus carnosus, Leuconostoc mesenteroides ssp cremoris, Pediococcus acidolactici, Pediococcus cerevisiae, Bifidobacterium bifidus, Bifidobacterium longum, Brevibacterium linens, Propionibacterium shermanii, Propionibacterium arabinosum, Penicillium roquefortii, Penicillium camembertii, or Staphylococcus xylosus.

A subject composition, which may be a pet food, treat, snack, supplement, or drink, can comprise a probiotic content of at least about 1, 2, 3, 4, 5, 6, 7, 8, 9, 10, 12, 14, 16, 18, 20 billion colonies forming units (CFU). A subject composition can comprise a probiotic content of at least about 1, 2, 3, 4, 5, 6, 7, 8, 9, 10, 12, 14, 16, 18, 20 billion colony forming units (CFU). A subject composition can comprise a probiotic content of at least about 2 billion CFU/kg, 4 billion CFU/kg, 6 billion CFU/kg, 8 billion CFU/kg, 10 billion CFU/kg, 15 billion CFU/kg, 20 billion CFU/kg, 25 billion CFU/kg, 30 billion CFU/kg, 35 billion CFU/kg, 40 billion CFU/kg. In some instances the composition comprises a probiotic content of 20 billion CFU/kg of the subject composition. In some instances, the composition comprises a probiotic content of 10 billion colonies.

Pet Treat Formulations

In certain embodiments, the pet food composition can be a treat. Treats include compositions that are given to an animal to entice the animal to eat during a non-meal time, for example, dog bones for canines Treats may be nutritional wherein the composition includes one or more nutrients and may have a food-like composition. Non-nutritional treats encompass any other treats that are non-toxic. The composition or components are coated onto the treat, incorporated into the treat, or both. Treats of the invention can be prepared by an extrusion or baking process similar to those used for dry food. Other processes also may be used to either coat the composition on the exterior of existing treat forms or inject the composition into an existing treat form.

In certain embodiments, the edible composition can be a toy. Toys include chewable toys such as artificial bones. The at least one pyruvate can form a coating on the surface of the toy or on the surface of a component of the toy, be incorporated partially or fully throughout the toy, or both. In one embodiment, the one or more pyruvate is orally accessible by the intended user. The chewable toy can comprise about 0.05 to 5 wt % of leucine and/or about 0.005 to 1 wt % of one or more leucine metabolites; and about 0.0005 to 0.05 wt % of a sirtuin activator. The chewable toy can comprise: at least about 50 mg of leucine and/or at least about 5 mg of one or more leucine metabolites; and at least about 1 mg of a sirtuin activator. There are a wide range of suitable toys currently marketed, for example, U.S. Pat. No. 5,339,771, U.S. Pat. No. 5,419,283, and references disclosed therein. This invention provides both partially consumable toys, for example, toys including plastic components, and fully consumable toys, for example, rawhides and various artificial bones.

Pet Supplement Formulations

The invention provides for pet supplements that can be in the form of pills, tablets, chews liquids or powders, or the like that are suitable for ingestion by a pet. Supplements include, for example, a feed used with another feed to improve the nutritive balance or performance of the total. Supplements include compositions that are fed undiluted as a supplement to other feeds, offered free choice with other parts of an animal's ration that are separately available, or diluted and mixed with an animal's regular feed to produce a complete feed. AAFCO, for example, provides a discussion relating to supplements in the Official Publication of The Association of American Feed Control Officials, Inc. (2009). Supplements may be in various forms including, for example, powders, liquids, syrups, pills, encapsulated compositions, and the like. In other embodiments, a pet supplement can be in the form of a powder or liquid that can be mixed in with a pet's daily meals. The powder or liquid can be packaged in individual packets or bottles such that one packet or bottle corresponds to a half or all of a recommended dosage of leucine, a leucine metabolite, and/or a sirtuin activator.

The tablets can be uncoated or coated by known techniques to delay disintegration and absorption in the gastrointestinal tract and thereby provide a sustained action over a longer period. For example, a time delay material such as glyceryl monostearate or glyceryl distearate can be employed. Formulations for oral use can also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example, peanut oil, liquid paraffin or olive oil.

In one embodiment, the composition may include a solubilizer to ensure good solubilization and/or dissolution of the compound of the present invention and to minimize precipitation of the compound of the present invention. This can be especially important for compositions for non-oral use, e.g., compositions for injection. A solubilizer may also be added to increase the solubility of the hydrophilic drug and/or other components, such as surfactants, or to maintain the composition as a stable or homogeneous solution or dispersion.

The compositions described herein can also be formulated as extended-release, sustained-release or time-release such that one or more components are released over time. Delayed release can be achieved by formulating the one or more components in a matrix of a variety of materials or by microencapsulation. The compositions can be formulated to release one or more components over a time period of 4, 6, 8, 12, 16, 20, or 24 hours. The release of the one or more components can be at a constant or changing rate.

Using the controlled release dosage forms provided herein, the one or more cofactors can be released in its dosage form at a slower rate than observed for an immediate release formulation of the same quantity of components. In some embodiments, the rate of change in the biological sample measured as the change in concentration over a defined time period from administration to maximum concentration for an controlled release formulation is less than about 80%, 70%, 60%, 50%, 40%, 30%, 20%, or 10% of the rate of the immediate release formulation. Furthermore, in some embodiments, the rate of change in concentration over time is less than about 80%, 70%, 60%, 50%, 40%, 30%, 20%, or 10% of the rate for the immediate release formulation.

Examples of suitable fillers for use in the compositions and dosage forms disclosed herein include, but are not limited to, talc, calcium carbonate (e.g., granules or powder), microcrystalline cellulose, powdered cellulose, dextrates, kaolin, mannitol, silicic acid, sorbitol, starch, pre-gelatinized starch, and mixtures thereof

When aqueous suspensions and/or elixirs are desired for oral administration, the active ingredient therein may be combined with various sweetening or flavoring agents, coloring matter or dyes and, if so desired, emulsifying and/or suspending agents, together with such diluents as water, ethanol, propylene glycol, glycerin and various combinations thereof

Pet Drinks

The present invention also provides for drink compositions for pet animals that provides a healthy, nutritional drink, in which the components can be easily adjusted to specific needs and which is palatable to the animals. The drinks can comprise the subject compositions, such as leucine, a leucine metabolite, and/or a sirtuin activator.

The drink composition according to the invention has three major components, an olfactory component (i.e., smell), a palatability component (i.e., taste), and a health component (i.e., pure and/or fortified water). Flavors, both natural and synthetic, may be used to promote the olfactory component of the invention. Beef, fish, chicken, turkey, liver and cat nip are flavors that may be used as olfactory and/or palatability enhancers. Other flavors, both natural and synthetic, may used to promote the palatability of the invention. Dextrose (D-glucose), fructose and mixed long chain polysaccharides are the carbohydrate palatability enhancers. Sodium pyrophosphate, sodium acid pyrophosphate, sodium tripolyphosphate, tetrasodium pyrophosphate, sodium polyphosphate, sodium tripolyphosphate, phosphoric acid, citric acid, and potassium citrate are noncarbohydrate palatability enhancers that may be in the drinks

Vitamins are major health components of the liquid drinks Vitamins in the liquid drinks may include both water soluble and water dispersible vitamins. The antioxidant vitamins C, E, A and alpha-tocopherol, as well as vitamin B12, vitamin D, folic acid, D-biotin, cyanocobalamin, niacinamide (B3), thiamin, riboflavin, pyridoxin (B6), menadione (K3), beta-carotene, calciumpantothenate, choline, and inositol may provide added nutrients to the liquid pet drinks. The concentration of the vitamins may be adjusted to deliver between 2% to 200% of the recommended daily requirements. The dog drink may be formulated with the necessary daily vitamin requirements for a 14 kg dog in 500 milliliters. The cat drink may be formulated with the necessary daily vitamin requirements for a 3 kg cat in 125 milliliters.

The drink of the invention may include mineral and electrolyte supplements. Zinc, iron, calcium, manganese, copper, iodine, sodium, and potassium may be added to the liquid drinks as mineral and electrolyte supplements. Additionally, the invention may include amino acid supplements. Alanine, arginine, aspartic acid, asparagine, cysteine, glutamic acid, glycine, glutamine, histidine, isoleucine, leucine, lysine, methionine, phenylalanine, proline, serine, taurine, threonine, tryptophan, tyrosine, and valine are amino acids that may be added to the invention.

The drink of the invention may include potassium sorbate and sodium benzoate as preservatives, and may also use low levels of carbon dioxide for improving shelf life and in limiting biological growth.

Pet Food Formulations

The invention provides for pet foods that can be in dry, moist or wet form using one or more food preparation processes. Foods of any consistency or moisture content are contemplated, e.g., the compositions of the present invention may be, for example, a dry, moist or semi-moist animal food composition. “Semi-moist” refers to a food composition containing from about 25 to about 35% moisture. “Moist” food refers to a food composition that has a moisture content of about 60 to 90% or greater. “Dry” food refers to a food composition with about 3 to about 11% moisture content and is often manufactured in the form of small bits or kibbles. Also contemplated herein are compositions that may comprise components of various consistency as well as components that may include more than one consistency, for example, soft, chewy meat-like particles as well as kibble having an outer cereal component and an inner cream component as described in, e.g., U.S. Pat. No. 6,517,877. The kibble may then be dried and optionally coated with one or more topical coatings known by those skilled in the art, for example, flavors, fats, oils, powders, and the like.

Kibble-type animal feeds, such as dog and cat foods, can be dried, ready-to-eat pet food products. The kibbles can be formed by an extrusion process where the kibble raw materials are extruded under heat and pressure to form the pelletized kibble form or core. Extrusion technology can provide an inexpensive and efficient method for formulating animal feed kibbles, such as those having a starch matrix. During the extrusion process, the kibble raw materials, which can comprise the starch matrix, typically results in the starch matrix becoming gelatinized under the extrusion conditions, forming a gelatinized starch matrix.

A process of manufacture of the pet food product can generally include mixing components to form a core material mixture, extruding the core material mixture to form a core pellet, drying the core pellet, and optionally applying a dusting component to the dried core pellet to form a food pellet, and packaging the food pellets. In one embodiment, the food pellet can be the final desired food product. In one embodiment, the food pellet can undergo dusting steps to form the food product as desired.

Ground animal proteinaceous tissues can be mixed with the other ingredients such as fish oils, cereal grains, balancing ingredients, special purpose additives, such as the leucine, a leucine metabolite, a sirtuin activator, other vitamin and mineral mixtures, inorganic salts, cellulose and beet pulp, bulking agents, and the like, and water in amounts sufficient for processing. These ingredients are mixed in a vessel suitable for heating while blending the components. Heating of the mixture is effected using any suitable manner, for example, direct steam injection or using a vessel fitted with a heat exchanger. Following the addition of the last ingredient, the mixture is heated to a temperature of about 50° F. to about 212° F. Temperatures outside this range are acceptable but may be commercially impractical without use of other processing aids. When heated to the appropriate temperature, the material will typically be in the form of a thick liquid. The thick liquid is filled into cans. A lid is applied, and the container is hermetically sealed. The sealed can is then placed into conventional equipment designed to sterilize the contents. Sterilization is usually accomplished by heating to temperatures of greater than about 230° F. for an appropriate time depending on the temperature used, the composition, and similar factors. The compositions of the present invention can be added to the food compositions before, during, or after preparation.

Food compositions may be prepared in a dry form using conventional processes known to skilled artisans. Typically, dry ingredients such as animal protein, plant protein, grains, and the like are ground and mixed together. Moist or liquid ingredients, including fats, oils, animal protein, water and the like are then added to and mixed with the dry mix. The mixture is then processed into kibbles or similar dry pieces. Kibble is often formed using an extrusion process in which the mixture of dry and wet ingredients is subjected to mechanical work at a high pressure and temperature and forced through small openings and cut off into kibble by a rotating knife. The wet kibble is then dried and optionally coated with one or more topical coatings such as flavors, fats, oils, powders, and the like. Kibble also can be made from the dough using a baking process, rather than extrusion, wherein the dough is placed into a mold before dry-heat processing. The food compositions can be in the form of a treat using an extrusion or baking process similar to those described above for dry food or a toy such as those disclosed in U.S. Pat. Nos. 5,339,771 and 5,419,283. The compositions of the present invention can be added to the food compositions before, during, or after preparation.

In many applications, starch can be added to the protein component of the core feed to improve stability, such as by holding the components in the kibble form. In certain applications, it may be desirable to provide a kibble that is substantially free of starch, using, for example, techniques described in US Patent Application No. 2011/0027416, incorporated herein by reference in its entirety. Thus, one embodiment of the present disclosure provides a protein-based core matrix, wherein the protein-based core is substantially free of a gelatinized starch matrix. Specific embodiments may comprise a protein-based core that has less than 5%, 2%, 1%, or even 0.5% by weight of gelatinized starch. Still other embodiments, the protein-based core matrix may be essentially free of gelatinized starch. As used herein, the term “essentially free” when used in reference to concentration of a specific component in a composition means less than a measurable amount using methods of concentration measurements common in the art.

As contemplated herein, the compositions of the present invention are can be nutritionally complete and balanced pet food compositions (also referred to herein simply as “nutritionally complete pet food compositions”). Nutrients and ingredients as well as others suitable for animal feed compositions, and recommended amounts thereof, may be found, for example, in the Official Publication of the Associate of American Feed Control Officials (“AAFCO”), Inc., Nutrient Requirements of Dogs and Cats, 2006. For example, nutritionally complete foods may contain protein, fat, carbohydrate, dietary fiber, amino acids, minerals, vitamins, and other ingredients in amounts known by those of skill in the art.

Protein may be supplied by any of a variety of sources known by those skilled in the art, including plant sources, animal sources, or both. Animal sources include, for example, meat, meat by-products, seafood, dairy, eggs, etc. Meats include, for example, the flesh of poultry, fish, and mammals (e.g., cattle, pigs, sheep, goats, and the like). Meat by-products include, for example, lungs, kidneys, brain, livers, and stomachs and intestines (freed of all or essentially all their contents). The protein can be intact, almost completely hydrolyzed, or partially hydrolyzed.

Fat can be supplied by any of a variety of sources known by those skilled in the art, including meat, meat by-products, fish oil, and plants. Plant fat sources include wheat, flaxseed, rye, barley, rice, sorghum, corn, oats, millet, wheat germ, corn germ, soybeans, peanuts, and cottonseed, as well as oils derived from these and other plant fat sources. A fat component can be used in some embodiments. The fat component can comprise poultry fat, chicken fat, turkey fat, pork fat, lard, tallow, beef fat, vegetable oils, corn oil, soy oil, cottonseed oil, palm oil, palm kernel oil, linseed oil, canola oil, rapeseed oil, fish oil, menhaden oil, anchovy oil, and/or olestra.

Carbohydrate may be supplied by any of a variety of sources known by those skilled in the art, including oat fiber, cellulose, peanut hulls, beet pulp, parboiled rice, corn starch, corn gluten meal, and any combination of those sources. Grains supplying carbohydrate include, but are not limited to, wheat, corn, barley, and rice. Carbohydrate content of foods may be determined by any number of methods known by those of skill in the art. Generally, carbohydrate percentage may be calculated as nitrogen free extract (“NFE”), which may be calculated as follows: NFE=100%−moisture %−protein %−fat %−ash %−crude fiber %.

Fatty acids for inclusion in the compositions of the present invention can include omega 3 fatty acids such as docosahexanenoic acid (DHA), eicosapentaenoic acid (EPA), alpha-linolenic acid (ALA), octadecatetraenoic acid (stearidonic acid) or mixtures thereof.

Dietary fiber refers to components of a plant which are resistant to digestion by an animal's digestive enzymes. Dietary fiber includes soluble and insoluble fibers. Soluble fiber are resistant to digestion and absorption in the small intestine and undergo complete or partial fermentation in the large intestine, e.g., beet pulp, guar gum, chicory root, psyllium, pectin, blueberry, cranberry, squash, apples, oats, beans, citrus, barley, or peas. Insoluble fiber may be supplied by any of a variety of sources, including cellulose, whole wheat products, wheat oat, corn bran, flax seed, grapes, celery, green beans, cauliflower, potato skins, fruit skins, vegetable skins, peanut hulls, and soy fiber. Crude fiber includes indigestible components contained in cell walls and cell contents of plants such as grains, e.g., hulls of grains such as rice, corn, and beans.

Amino acids, including essential amino acids, may be added to the compositions of the present invention as free amino acids, or supplied by any number of sources, e.g., crude protein, to the compositions of the present invention. Essential amino acids are amino acids that cannot be synthesized de novo, or in sufficient quantities by an organism and thus must be supplied in the diet. Essential amino acids vary from species to species, depending upon the organism's metabolism. For example, it is generally understood that the essential amino acids for dogs and cats (and humans) are phenylalanine, leucine, methionine, lysine, isoleucine, valine, threonine, tryptophan, histidine and arginine. In addition, taurine, while technically not an amino acid but a derivative of cysteine, is an essential nutrient for cats.

The compositions of the present invention may also contain one or more minerals and/or trace elements, e.g., calcium, phosphorus, sodium, potassium, magnesium, manganese, copper, zinc, choline, or iron salts, in amounts required to avoid deficiency and maintain health. These amounts are known by those of skill in the art, for example, as provided in the Official Publication of the Associate of American Feed Control Officials, Inc. (“AAFCO”), Nutrient Requirements of Dogs and Cats, 2006.

The compositions of the present invention may also include vitamins in amounts required to avoid deficiency and maintain health. These amounts, and methods of measurement are known by those skilled in the art. For example, the Official Publication of the Associate of American Feed Control Officials, Inc. (“AAFCO”), Nutrient Requirements of Dogs and Cats, 2006 provides recommended amounts of such ingredients for dogs and cats. As contemplated herein, useful vitamins may include, but are not limited to, vitamin A, vitamin B1, vitamin B2, a sirtuin activator, vitamin B12, vitamin C, vitamin D, vitamin E, vitamin H (biotin), vitamin K, folic acid, inositol, niacin, and pantothenic acid.

The compositions of the present invention may additionally comprise additives, stabilizers, fillers, thickeners, flavorants, palatability enhancers and colorants in amounts and combinations familiar to one of skill in the art.

In one embodiment, the compositions of the present invention are provided as a food, e.g., a nutritionally complete pet food composition. In another embodiment, the compositions of the present invention may be in the form of a treat, snack, supplement, or partially or fully edible toy. Such items for consumption by a pet are known to those skilled in the art, and can include, for example, compositions that are given to an animal to eat during non-meal time, e.g., a dog biscuits, edible chew toys, etc.

The binder component in any pet food composition described herein can comprise any of the following or combinations of the following materials: monosaccharides such as glucose, fructose, mannose, arabinose; di- and trisaccharides such as sucrose, lactose, maltose, trehalose, lactulose; corn and rice syrup solids; dextrins such a corn, wheat, rice and tapioca dextrins; maltodextrins; starches such as rice, wheat, corn, potato, tapioca starches, or these starches modified by chemical modification; oligosaccharides such as fructooligosccharides, alginates, chitosans; gums such as carrageen, and gum arabic; polyols such as glycerol, sorbitol, mannitol, xylitol, erythritol; esters of polyols such as sucrose esters, polyglycol esters, glycerol esters, polyglycerol esters, sorbitan esters; sorbitol; molasses; honey; gelatins; peptides; proteins and modified proteins such as whey liquid, whey powder, whey concentrate, whey isolate, whey protein isolate, high lactose whey by-product, such as DAIRYLAC® 80 from International Ingredient Corporation, meat broth solids such as chicken broth, chicken broth solids, soy protein, and egg white. These aforementioned binder components can be used in combination with water, especially when added. The binder material can be dissolved or dispersed in water, forming a liquid mixture or solution, which can then be applied over the surface of the core. The liquid mixture can facilitate both even dispersion of the binder component over the core surface and the interaction between the core surface and the protein component being applied to the surface of the core. In one embodiment, the liquid mixture can be an about 20% liquid mixture of binder component, which can be added to the kibble at 5% to 10% by weight of the kibble, which, on a dry matter basis, becomes about 1% to 2% by weight of the kibble.

In embodiments when a binder component is used, keeping the binder component on the surface of the core can be done, thus preventing, or at least attempting to minimize, absorption of the binder towards and into the core. In one embodiment, additives can be added to increase the viscosity of the binder solution. Those additives can be corn starch, potato starch, flour, and combinations and mixtures thereof. These additives can assist in keeping the binder component on the surface of the kibble to prevent or minimize absorption from the surface towards and into the core. In another embodiment, varying the temperature of the binder solution to thicken the solution can be done. For example, when using egg white as a binder component, denaturization of the proteins of the egg whites can create a gel-like solution. This formation of a gel-like solution can occur around 80° C., so in one embodiment raising the temperature of the binder solution to 80° C. can be performed. Additionally, the temperature of the core can be increased to also assist in minimizing the absorption of the binder towards the core. In another embodiment, additives and temperature variation as just described can also be done in combination.

Thus, in one embodiment, the binder component can act as a glue, or adhesive material, for the protein component to adhere to the core. In one embodiment, the protein component can be a solid ingredient at less than 12% moisture, or water, content, and the binder component can be a liquid. In one embodiment, the binder component can be applied to or layered onto the core to act as the glue for the protein component, which can then be applied to or layered onto the core with binder component. In another embodiment, the protein component as a solids ingredient can be mixed with the binder component, and then the mixture can be applied to or layered onto the core.

In one embodiment, lipids and lipid derivatives can also be used as binder components. Lipids can be used in combination with water and/or other binder components. Lipids can include plant fats such as soybean oil, corn oil, rapeseed oil, olive oil, safflower oil, palm oil, coconut oil, palm kernel oil, and partially and fully hydrogenated derivatives thereof; animal fats and partially and fully hydrogenated derivatives thereof; and waxes.

Coatings

In some embodiments, the subject compositions can have a modular structure. For example, the subject composition can have an inner and an outer portion. The inner portion can be initially formed, and the outer portion can be coated on or dusted on to the inner portion. The coating and/or modular structure of the subject composition can allow for increased variety in food selection while maintaining an efficient and cost-effective manufacturing process. The subject compositions can be manufactured such that the one or more active ingredients of the subject composition are in the outer portion or coating of the composition.

In some embodiments, a pet food composition can comprise an inner layer comprising kibble; an outer layer; about 0.05 to 5% of leucine and/or about 0.005 to 1% of one or more leucine metabolites by weight of the pet food composition; and about 0.0005 to 0.05% of a sirtuin activator by weight of the pet food composition. Optionally, the amount of leucine, leucine metabolite, and/or sirtuin activator can be any other amount described herein.

In other embodiments, a pet food or supplement composition comprises an inner layer comprising kibble; an outer layer; and at least about 50 mg of leucine and/or at least about 5 mg of one or more leucine metabolites by weight of the pet food composition; and at least about 1 mg of a sirtuin activator by weight of the pet food composition. Optionally, the amount of leucine, leucine metabolite, and/or sirtuin activator can be any other amount described herein.

The inner portion may be in the form of a paste. By paste it is meant a viscous composition that retains its position within the outer portion but is soft and can coat (at least transiently) the teeth of the animal when consumed. The paste may be of a similar consistency to, for example, toothpaste. The outer portion is in the form of a tube.

Layering of a protein component, or any of the other components as described herein, as a coating on a core, as described herein, can also alter the aroma profile of a coated kibble and result in a coated kibble having different aroma profiles than typical pet food. Certain embodiments of coated kibbles as disclosed herein may contain specific compounds and components that can give the pet food desirable aromas. These compounds and components can cause changes in the aroma profile, or aroma attribute changes, which can result in improved animal preference, or animal acceptance or preference, using embodiments of a coated kibble as disclosed herein. Without being bound by theory, it is thought that these aroma attribute changes contribute to the improved preference of a coated kibble wherein the coating comprises a protein component, a non-limiting example such as chicken by-product meal, layered onto a kibble core. Previous consumer research has suggested that human-like aromas on pet food could be perceived as improvements in products.

Thus, one non-limiting example of an embodiment of the present invention relates to a coated kibble, and a method of delivering a coated kibble, having an aroma profile, an analyte concentration, and an aroma correlation, wherein the aroma correlation relates the aroma profile comprising an analyte concentration to the increase in animal preference. Additionally, another embodiment relates to a coated kibble having an aroma profile, an analyte concentration, and thus an aroma correlation. With these embodiments, animal preference (PREF) response data, or animal acceptance or preference, can be correlated with the aroma profile and analyte concentration, as disclosed herein. Thus, in one embodiment, aroma analyte profiles and concentrations can correlate to positive, or increased, animal preference response data. Additionally, in one embodiment, the coated kibble comprises an animal preference enhancing amount of an analyte. The animal preference enhancing amount of the analyte can be within the coating, within the core, and combinations and mixtures of these. In another embodiment, a method of enhancing the animal preference of a pet food comprises delivering an animal preference enhancing amount of an analyte in a pet food is disclosed. As used herein, animal preference enhancing amount means an amount that increases the animal preference response, whether ratio percent converted intake or ratio first bite, or both of these. The ratio first bite can be an alternative measurement to assess the animal preference response.

The aroma profile, including analyte concentration, can be determined in accordance with the method as disclosed hereinafter, using Solid Phase MicroExtraction Gas Chromatography/Mass Spectrometry (SPME-GC-MS) to analyze pet food samples for compounds associated with the aroma. The area under the curve was measured as the SPME analysis number or count.

One embodiment of the present invention relates to a coated kibble and a method of delivery thereof wherein the coated kibble has a particular aroma profile. A non-limiting example of a coated kibble comprises a core comprising a carbohydrate source, a protein source, a fat source, and other ingredients, all as disclosed herein, and a coating comprising a protein component, a binder component, a palatant component, a fat component, and other components. In this embodiment, an aroma profile of the coated kibble can be generated and analyzed showing specific analyte concentrations the aroma. Concentrations can be determined for each of the analytes. The concentration of the analytes can then be correlated with PREF response data that was gathered for each of the embodiments to show an aroma correlation with the PREF response data. Thus, in one embodiment, an increase in particular analytes present in the aroma can drive up, or increase the PREF response data, meaning a greater PREF response, resulting in higher animal preference or acceptance.

In one embodiment, the analytes 2-Piperidione, 2,3 pentanedione, 2-ethyl-3,5-dimethypyrazine, Furfural, Sulfurol, Indole, and mixtures and combinations of these, can be elevated or representative of families with elevated levels when compared to off the shelf pet food. Thus, in one embodiment, a coated kibble comprising particular concentrations of the analytes 2-Piperidione, 2,3 pentanedione, 2-ethyl-3,5-dimethypyrazine, Furfural, Sulfurol, Indole, and mixtures and combinations of these, increases PREF response. Thus, an animal preference enhancing amount of the analytes 2-Piperidione, 2,3 pentanedione, 2-ethyl-3,5-dimethypyrazine, Furfural, Sulfurol, Indole, and mixtures and combinations of these, can be present in one embodiment of the coated kibble. This animal preference enhancing amount of the analytes can increase the PREF response. In one embodiment, the Ratio Percent Converted Intake (PCI) can increase with an animal preference enhancing amount of the analytes 2-Piperidione, 2,3 pentanedione, 2-ethyl-3,5-dimethypyrazine, Furfural, Sulfurol, Indole, and mixtures and combinations of these. In another embodiment, the ratio first bite can increase with an animal preference enhancing amount of the analytes 2-Piperidione, 2,3 pentanedione, 2-ethyl-3,5-dimethypyrazine, Furfural, Sulfurol, Indole, and mixtures and combinations of these.

Thus, one embodiment of the present invention relates to a coated kibble comprising an enriched amount, or an animal preference enhancing amount, of the analytes 2-Piperidione, 2,3 pentanedione, 2-ethyl-3,5-dimethypyrazine, Furfural, Sulfurol, Indole, and mixtures and combinations of these. Another embodiment includes a method of delivering a coated kibble comprising an animal preference enhancing amount of the analytes 2-Piperidione, 2,3 pentanedione, 2-ethyl-3,5-dimethypyrazine, Furfural, Sulfurol, Indole, and mixtures and combinations of these.

Another embodiment of the present invention relates to a method of enhancing the animal preference of a pet food comprising delivering an animal preference enhancing amount of an analyte in a pet food. The method can include providing a pet food, as disclosed herein, wherein the pet food comprises enriched amount, or an animal preference enhancing amount, of the analytes 2-Piperidione, 2,3 pentanedione, 2-ethyl-3,5-dimethypyrazine, Furfural, Sulfurol, Indole, and mixtures and combinations of these. The method can also comprise adding to pet food animal preference enhancing amounts of the analytes 2-Piperidione, 2,3 pentanedione, 2-ethyl-3,5-dimethypyrazine, Furfural, Sulfurol, Indole, and mixtures and combinations of these.

Shelf Life

In other embodiments, the subject compositions can be manufactured with a coating on the surface of the composition that increases the shelf life of the subject composition. The coating can form a gas (such as oxygen), water or moisture barrier that allows the subject composition to withstand a greater range of environmental conditions, such as wet, moist, humid, dry, cold, or hot environments, and oxidation.

A pet food composition for increasing the shelf life of a physically discrete dry pet food can comprise a coat covering the physically discrete pet food composition comprising a polymer film, wherein the film or an agent in the film protects the composition from oxidation decomposition and/or protects the composition from bacterial growth, wherein the film comprises a starch/synthetic polymer selected from the group consisting of starch/polyethylene, and starch/low-density polyethylene, and wherein the thickness of said coat is 1-2000 microns; about 0.05 to 5 wt % of leucine and/or about 0.005 to 1 wt % of one or more leucine metabolites; and about 0.0005 to 0.05 wt % of a sirtuin activator. Optionally, the amount of leucine, leucine metabolite, and/or sirtuin activator can be any other amount described herein.

A method for increasing the shelf life of a physically discrete dry pet food composition can comprise coating the physically discrete pet food composition with a polymer film, wherein the film or an agent in the film protects the composition from oxidation decomposition and/or protects the composition from bacterial growth, wherein the film comprises a starch/synthetic polymer selected from the group consisting of starch/polyethylene, and starch/low-density polyethylene, and wherein the thickness of said coating is 1-2000 microns, wherein the pet food composition comprises about 0.05 to 5 wt % of leucine and/or about 0.005 to 1 wt % of one or more leucine metabolites and about 0.0005 to 0.05 wt % of a sirtuin activator. Optionally, the amount of leucine, leucine metabolite, and/or sirtuin activator can be any other amount described herein.

The external portion of the discrete pet food composition such as a kibble, solid, treat or supplements or even a “chunk” in a canned food “chunk and gravy” diet can be used a carrier for the benefit agent. However the benefit agent is not applied directly to the surface of the discrete portion of the pet food composition. Rather, the benefit agent is delivered to the solid surface in the matrix of a film which is physically stable and capable of being expressed on the surface of the physically discrete portion of the pet food composition. The benefit agent can increase the shelf life of the food product.

The chemical used in coating can be physically stable during the process of its application and also stable during its lifetime on the pet food composition surface while being subjected to any further processing steps. It can remain essentially chemically inert with the surface, itself or its environment but can be somewhat reactive as long as its function in the system is not significant jeopardized. Its compatibility with the oral cavity and digestive tract of the pet can also be present. Examples of these polymers include zein, casein, starch(es), cellulose(s), gum(s), gelatin, starch/synthetic polymer(s), e.g. starch/low density polyethylene, and the like. The polymer can have the attribute of rapid dissociation in the oral cavity, particularly in the presence of saliva. The thickness of the coating is not as important. It can vary from about 1 to about 2000 microns, or from about 2 to about 1000 microns, as long as the function of the film is maintained.

In other embodiments of the invention, pet food and treat compositions that include leucine, a leucine metabolite, and/or a sirtuin activator can be prepared in methods that increase the shelf-life of the pet food or treat by manufacturing the pet food across multiple locations.

Pet food compositions are subject to deterioration in transit, although deterioration is not usually a problem as long as the finished product is not shipped over long distances. However, many manufacturers in the United States recognize a growing demand for premium and super-premium pet food products in developing international markets. Typically, within such markets, manufacturers have not installed the full capacity (meat slurry) plant systems which are required to process the pet food compositions. Thus, to meet the demand, manufacturers face the choice of exporting finished product over long distances from existing full capacity plants, or investing in full capacity plants in the newly opened markets.

A method for making kibble at one location and finishing the pet food at another location can comprise forming a dry, stable intermediate of the pet food composition at a first location; finishing the dry, stable intermediate pet food composition to form a finished pet food composition at a second location remote from the first location, wherein said finished pet food composition contains about 0.05 to 5 wt % of leucine and/or about 0.005 to 1 wt % of one or more leucine metabolites and about 0.0005 to 0.05 wt % of a sirtuin activator. Optionally, the amount of leucine, leucine metabolite, and/or sirtuin activator can be any other amount described herein.

In an exemplary embodiment of the invention, a process for making a stable intermediate pet food composition includes the steps of combining a plurality of ingredients specified by a basal pet food formula to form a basal mixture, and processing the basal mixture to produce a dry, stable intermediate pet food composition containing meat. In one embodiment, the step of processing the basal mixture includes extruding the basal mixture, cutting the extruded basal mixture into flakes, and drying the flakes to a moisture content of less than about 10% by weight. The intermediate pet food composition, in the form of dried flakes, is then packaged in storage containers and stored until needed to produce a finished pet food composition. In one embodiment, the process further includes the steps of transporting the pet food composition to a processing facility, and re-processing the dry flakes to produce the finished product.

In one embodiment, the plant further has packaging capability and the process further includes the steps of packaging the dry, stable intermediate pet food composition in containers suitable for storage, and transporting the pet food composition for finishing to a processing facility remote from the plant. In this embodiment, the dry, stable intermediate pet food composition is packaged and shipped to the remote processing facility. During shipping and storage, the dry, stable intermediate pet food composition resists deterioration. At the remote processing facility, the flakes are re-processed to produce a finished pet food composition, and then packaged for local distribution.

Palatability

In some embodiments, the subject compositions can include one or more palatability enhancers to improve the taste of the subject composition as perceived by the subject, such as a dog, cat, or any other domestic animal.

Pet food manufacturers have a long-standing desire to provide foods which combine high nutritional value, and resistance to decomposition and bacterial contamination, with low production costs. In addition, and with particular regard to cat foods, pet food manufacturers desire a high degree of palatability which can be attained at low cost.

A palatant component can be used in some embodiments. The palatant can comprise chicken flavor, such as liquid digest derived from chicken livers, which can be approximately 70% water and chicken liver digests. A palatant component as used herein means anything that is added to the animal feed for the primary purpose of improving food acceptance, or preference, by the animal. A palatant component, which can also be considered a flavor, a flavoring agent, or a flavoring component, can include a liver or viscera digest, which can be combined with an acid, such as a pyrophosphate. Non-limiting examples of pyrophosphates include, but are not limited to, disodium pyrophosphate, tetrasodium pyrophosphate, trisodium polyphosphates, tripolyphosphates, and zinc pyrophosphate. The palatant component can contain additional palatant aids, non-limiting examples of which can include methionine and choline. Other palatant aids can include aromatic agents or other entities that drive interest by the animal in the food and can include cyclohexanecarboxylic acid, peptides, monoglycerides, short-chain fatty acids, acetic acid, propionic acid, butyric acid, 3-methylbutyrate, zeolite, poultry hydrolysate, tarragon essential oil, oregano essential oil, 2-methylfuran, 2-methylpyrrole, 2-methyl-thiophene, dimethyl disulfide, dimethyl sulfide, sulfurol, algae meal, catnip, 2-Piperidione, 2,3 pentanedione, 2-ethyl-3,5-dimethypyrazine, Furfural, Sulfurol, and Indole. In addition, various meat based flavorants or aroma agents can be used, non-limiting examples include meat, beef, chicken, turkey, fish, cheese, or other animal based flavor agents.

The pet food can have a concentration of butyric acid of 5 to 1000 ppm, of 6 to 200 ppm, and/or a concentration of 3-methylbutyric acid of 4 to 500 ppm, of 5 to 200 ppm, and/or salt thereof. Alternatively, a pet food can be provided that contains liver but not rumen and that has a concentration of butyric acid of 7 ppm to 10,000 ppm and/or a concentration of 3-methylbutyric acid of 4 ppm to 10,000 ppm, and/or salt thereof. The pet food can have a concentration of butyric acid of 10 to 1,000 ppm, of 12 to 500 ppm, and/or a concentration of 3-methylbutyric acid of 4 to 500 ppm, of 5 to 200 ppm, and/or salt thereof.

Preferred acids are selected from among acetic acid, propionic acid, isobutyric acid, 2-methylbutyric acid, pentanoic acid, hexanoic acid, 2-methylpentanoic acid, 4-methylpentanoic acid, 2-methyl-2-pentanoic acid and mixtures of these acids. These acids can be added in an additional amount exceeding the amount that is naturally contained in the pet food.

The palatability enhancer can include tetrasodium pyrophosphate and is formulated as a dry mixture in powdered, granulated or encapsulated form. Tetrasodium pyrophosphate is available in dry, powdered form from, for example, Solutia of St. Louis, Mo., and is combined with other dry ingredients including known palatability enhancing ingredients and preservatives. The tetrasodium pyrophosphate can be used in about 5% to about 1.0% by weight of the total composition.

Texture

In some embodiments of the invention, the pet foods and treats can have one or more textures, or specifically selected textures. For example, a pet food composition comprising leucine and/or leucine metabolites and a sirtuin activator can have a dual texture. Additionally, a wet pet food composition comprising leucine and/or leucine metabolites and a sirtuin activator can be manufactured such that the wet pet food mimics a texture that would be appealing to a pet. The wet pet food texture can be achieved by utilizing a structurant and/or bonding layers or compositions to each other.

In some embodiments, a pet food composition can comprise an outer layer joined to an inner layer, wherein the outer layer is harder than the inner layer; about 0.05 to 5 wt % of leucine and/or about 0.005 to 1 wt % of one or more leucine metabolites; and about 0.0005 to 0.05 wt % of a sirtuin activator. Optionally, the amount of leucine, leucine metabolite, and/or sirtuin activator can be any other amount described herein.

In some instances, the pet food product can be a shelf-stable dual texture multicomponent pet or animal food product containing a softer lipid based portion contained within a shell or harder matrix material portion having significantly improved palatability, as compared to mono-textured pet or animal food products.

One aspect of the invention provides a dual texture edible product having a lipid-containing softer portion and a cereal based harder portion. The softer component is can be a mixture of lipids and solids which forms a soft cream textured matrix. Since this soft matrix has minimal water content and very low water activity levels, it does not require harsh sterilization techniques for preservation, additional ingredients for moisture control, or antimicrobial/antimycotic agents for stabilization. The product of the present invention can be stable, while still maintaining superior feeding performance, even after one year. The cereal based component can be harder than the lipid-containing component. According to one embodiment, the cereal based component can form a shell that surrounds the soft component which is in the form of an inner portion.

One of the processes for forming the two textured pet food product is an injection molding process, in which the two different textures are created by the injection of the two components into the same mold. The mold can be in the shape of a chicken drumstick or any other desired shape. The ingredients for each of the components are fed into different extruders where the ingredients are mixed to form moldable masses. Each component is injected into the mold in which the mixture for the inner component is injected into the center portion of the mold and the outer component surrounds a portion of the inner component. The mold can be either heated and/or cooled to set the texture of product for possible down-stream processing, which includes, but is not limited to retorting, baking or irradiating.

Pet foods are generally manufactured and available in dry, moist and semi-moist formulations. Wet pet foods may be prepared from proteinaceous materials such as meat, including meat by-products or fish. Wet pet foods can further be formed from a combination of farinaceous materials, such as wheat or other grains, or proteinaceous materials. While flavorings and aroma modifiers are used to improve palatability, wet pet foods often suffer from lack of appeal to consumers.

The wet pet food product and methods of the present invention can comprise, consist of, or consist essentially of, the essential elements and limitations of the invention described herein, as well as any additional or optional ingredients, components, or limitations described herein or otherwise useful in wet pet food product intended for animal consumption.

The pet food product of the present invention is typically in the form of a wet pet food product. The wet pet food products of the present invention can be a semi-moist pet food products (i.e. those having a total moisture content of from 16% to 50%, by weight of the product), and/or a moist pet food products (i.e. those having a total moisture content of greater than 50%, by weight of the product). Unless otherwise described herein, semi-moist pet food product, and moist pet food products are not limited by their composition or method of preparation.

The wet pet food product can comprise an edible component that can comprise a textured material that can comprise a structurant. The textured material has a Moisture Content in the range of from about 16% to about 92%. The textured material can have a Moisture Content of at least about 20%, at least about 25%, at least about 30%, at least about 35%, at least about 40%, at least about 50%, at least about 60%, at least about 70%.

In some embodiments, a pet food composition can comprise a structurant for providing a textured appearance and feel; about 0.05 to 5 wt % of leucine and/or about 0.005 to 1 wt % of one or more leucine metabolites; and about 0.0005 to 0.05 wt % of a sirtuin activator. Optionally, the amount of leucine, leucine metabolite, and/or sirtuin activator can be any other amount described herein.

In other embodiments, a pet food composition can comprise a textured layer bonded to a base layer, wherein the textured layer comprises textured components bonded to the base layer; about 0.05 to 5 wt % of leucine and/or about 0.005 to 1 wt % of one or more leucine metabolites; and about 0.0005 to 0.05 wt % of a sirtuin activator. Optionally, the amount of leucine, leucine metabolite, and/or sirtuin activator can be any other amount described herein.

The textured material can be a cube, irregular, elongated, cylindrical, geometric shapes, axially elongated, and combinations thereof. The textured material can be processed by a variety of well-known means including steam tunnel, extrusion, freeze-texturization, baking, gelling, retort, microwave heating, and combinations thereof

The textured material can have a coating associated with the textured material. Additionally, the textured material can have a first layer bonded to the textured material. In an embodiment of the present invention the textured material can have a coating associated with the textured material and a first layer bonded to the textured material. The textured material can be a cube, irregular, elongated, cylindrical, geometric shapes, axially elongated, and combinations thereof. The textured material can be processed by a variety of well-known means including steam tunnel, extrusion, freeze-texturization, baking, gelling, retort, microwave heating, and combinations thereof.

The wet pet food product comprises an edible component comprising a textured material that may comprise a structurant. The textured material can have a first coating associated with the textured material. The first coating can comprise a binder.

Nonlimiting examples of binders include egg-based materials (including egg whites, preferably dried egg whites), undenatured proteins, food grade polymeric adhesives, gels, polyols, starches (including modified starches), gums, and mixtures thereof. Nonlimiting examples of disaccharides which may be utilized herein include sucrose, maltose, lactitol, maltitol, maltulose, and lactose.

The wet pet food product can comprise a base food. The base food can have a coating associated with the base food. The base food can comprise a structurant that is selected from the group consisting of animal protein, plant protein, farinaceous matter, vegetables, fruit, and combinations thereof

Additionally, the base food can have a first layer bonded to the base food. In an embodiment of the present invention the base food can have a coating associated with the base food and a first layer bonded to the textured material. The base food can be processed by a variety of well-known means including steam tunnel, extrusion, freeze-texturization, baking, gelling, retort, microwave heating, and combinations thereof. The structurant can be an extruded protein. The base food can be a cube, irregular, elongated, cylindrical, spherical, geometric shapes, axially elongated, and combinations thereof.

The wet pet food product can comprise a first layer. The first layer can comprise a plurality of particles. The plurality of particles can be adhered together to form a first layer. Methods of adhering the plurality of particles include, but are not limited to, compressing molding, shaking, extruding, heating, and combinations thereof. Wherein the particles can be physically and/or chemically adhere. The particles can be at least about 5 to about 600 microns in size, as measured in at least one axis.

The first layer can be bonded with the base food. For example in an embodiment having the first layer bonded with the base food a stable wet food product is formed. The methods of bonding the first layer with the base food of the present invention, include, but are not limited to, compressing, molding, shaking, extruding, heating, and combinations thereof. The resulting wet pet food product can have varying shapes, sizes and appearance. A coating can be used when a first layer is bonded with a base food.

The first layer and/or plurality of particles is selected from the group consisting of animal protein, plant protein, farinaceous matter, vegetables, fruit and combinations thereof. The first layer can be processed by a variety of well-known means including steam tunnel, extrusion, freeze-texturization, baking, gelling, retort, and combinations thereof. Examples of the first layer include steam tunnel meat, extruded meat, partially cooked meat, baked meat, gelled meat, retort processed meat and combinations thereof. The first layer can be shredded from about 2 millimeters to about 30 millimeters in size, as measured in at least one axis, from about 3 millimeters to about 20 millimeters in size, from about 6 millimeters to about 15 millimeters in size, as measured in at least one axis.

The animal protein may be derived from any of a variety of animal sources including, for example, muscle meat or meat by-product. Nonlimiting examples of animal protein include beef, pork, poultry, lamb, kangaroo, shell fish, crustaceans, fish, and combinations thereof including, for example, muscle meat, meat by-product, meat meal, or fish meal.

The plant protein may be derived from any of a variety of plant sources. Nonlimiting examples of plant protein include lupin protein, wheat protein, soy protein, and combinations thereof. A portion, or all of the plant protein when present, can be a texturized protein, for example a textured wheat protein.

The farinaceous matter is commonly known in the pet food industry. Nonlimiting examples of farinaceous matter include grains such as, rice, corn, milo, sorghum, barley, and wheat, and the like, pasta (for example, ground pasta), breading, soy, and combinations thereof

Vegetables are commonly known in the pet food industry. Nonlimiting examples of vegetables include peas, carrots, corn, potatoes, beans, cabbage, tomatoes, celery, broccoli, cauliflower, and leeks. Fruits are commonly known in the pet food industry. Nonlimiting examples include tomatoes, apples, avocado, pears, peaches, cherries, apricots, plums, grapes, oranges, grapefruit, lemons, limes, cranberries, raspberries, blueberries, watermelon, cantaloupe, mushmellon, honeydew melon, strawberries, banana, and combinations thereof

Low Carbohydrate Formulations

In other embodiments of the invention, pet foods and treats of the composition that include leucine, a leucine metabolite, and/or a sirtuin activator can be formulated to have a low amount of carbohydrates. These low carbohydrate pet food and treat compositions can facilitate weight loss in a pet.

A pet food composition can comprise less than about 19% on a dry weight basis of carbohydrate; about 0.05 to 5 wt % of leucine and/or about 0.005 to 1 wt % of one or more leucine metabolites; and about 0.0005 to 0.05 wt % of a sirtuin activator. Optionally, the amount of leucine, leucine metabolite, and/or sirtuin activator can be any other amount described herein.

Relatively low carbohydrate, relatively high protein and fat content pet food can be successfully extruded into a discrete particle, which is dimensionally stable. It has the physical attributes of typical commercial pet foods which include much higher levels of carbohydrate.

By “dimensionally stable” it is meant that the resulting extruded product when sufficiently dried has physical integrity i.e., not readily losing its shape or shedding significant amounts of fines, particularly when the food is in discrete particles such as kibbles, bits and the like in a bag filled with the materials. Additionally, such a food often does not readily retain its fat content in a cohesive manner, particularly when the fat is deposited on the exterior of the discrete particle. Non-adherence can be visually observed. These problems are further accentuated by using a high quantity of protein. Protein isolates, which are generally used when there is a high protein content, particularly the vegetable isolates, make it even more difficult to successfully extrude a dry pet food having discrete particles which are dimensionally stable.

Utilizing a standard single screw extruder with a preconditioner, under standard operating conditions dimensionally stable discrete particles of the pet food described herein were not obtainable. After much work, it was found that increasing the shear in the extruder created an extruded pet food which was processed into discrete particles which were dimensionally stable even with the relatively low levels of carbohydrate described herein. The increased shear produces a pet food discrete particle, which is generally of a higher density than the discrete particle produced under normal shear processing conditions. Increased shear during the processing can be produced by various means such as for example using cut flight screws, lobe locks, steam locks, and straight ribbed liners.

The pet foods included are those useful primarily for dogs and cats. These foods are high in protein and fat and contain a relatively small amount of carbohydrate as compared to protein and fat. The resulting pet foods are dry as opposed to a wet chunk and/or gravy. The carbohydrate content based on nitrogen free extract, “NFE”, is a minimum of zero, 5 or 7 with a maximum of about 22, 15 or 10 wt %. All numbers are on a dry matter basis.

Very little, if any, carbohydrate can be initially present in the food. The carbohydrate can enter the food as part of another source such as protein but also can be present through specifically added carbohydrate sources such as starches and grains. Examples of such carbohydrate sources include a starch such as corn starch or wheat starch or mixtures thereof and a grain which can be greater than 50% starch such as corn, sorghum, barley, wheat, rice and the like as well as mixtures thereof. A specific carbohydrate source such as a starch, however, is not necessary.

The protein can come from any source but because of the relatively low carbohydrate level, a protein source with low carbohydrates is particularly preferred. Examples of such protein sources are animal sources such as pork protein isolate and veal protein isolate and the like as well as vegetable sources such as soy protein isolate, corn gluten meal and the like.

Specified Composition Diet

In other embodiments of the invention, pet foods and treats of the composition that include leucine, a leucine metabolite, and/or a sirtuin activator can be formulated to have specified levels of protein, fat, and/or fiber. These formulations with specified levels of components can facilitate weight loss in a pet.

In some embodiments, a pet food composition comprises about 0.05 to 5 wt. % of leucine and/or about 0.005 to 1 wt % of one or more leucine metabolites on a dry matter basis; about 0.0005 to 0.05 wt. % of a sirtuin activator on a dry matter basis; about 26 to 35 wt. % of crude protein on a dry matter basis; about 7.5 to 8.5 wt. % of crude fat on a dry matter basis; about 20 to 30 wt. % of total dietary fiber on a dry matter basis; and about 10 to 20 wt. % of crude fiber on a dry matter basis. Optionally, the amount of leucine, leucine metabolite, and/or sirtuin activator can be any other amount described herein.

In another embodiment, the invention encompasses a feline pet food composition comprising: 0.05 to 5 wt. % of leucine and/or about 0.005 to 1 wt % of one or more leucine metabolites on a dry matter basis; about 0.0005 to 0.05 wt. % of a sirtuin activator on a dry matter basis; 30 wt. % to 37 wt. % of crude protein on a dry matter basis, 7.5 wt. % to 9 wt. % of crude fat on a dry matter basis, 30 wt. % to 35 wt. % of total dietary fiber on a dry matter basis, and 20 wt. % to 25 wt. % of crude fiber on a dry matter basis. Optionally, the amount of leucine, leucine metabolite, and/or sirtuin activator can be any other amount described herein.

Canine Food Compositions: Proportion of the composition (% of dry weight of Component composition or parts per million) Leucine and/or a leucine metabolite as described herein, a sirtuin activator as described herein, Protein 26 wt. % to 35 wt. % of crude protein on a dry matter basis or 28 wt. % to 33 wt. %, or 30 wt. % to 31 wt. %; or 26 wt. %, 26.5 wt. %, 27 wt. %, 27.5 wt. %, 28 wt. %, 28.5 wt. %, 29 wt. %, 29.5 wt. %, 30 wt. %, 30.5 wt. %, 31 wt. %, 31.5 wt. %, 32 wt. %, 32.5 wt. %, 33 wt. %, 33.5 wt. %, 34 wt. %, 34.5 wt. %, 35 wt. %, Crude Fat 7.5 wt. % to 8.5 wt. % of crude fat on a dry matter basis, or 7.6 wt. %, 7.7 wt. %, 7.8 wt. %, 7.9 wt. %, 8.0 wt. %, 8.1 wt. %, 8.2 wt. %, 8.3 wt. %, 8.4 wt. % Total 20 wt. % to 30 wt. % of total dietary fiber on a dry matter basis Dietary Fiber or 22 wt. % to 28 wt. %, or 24 wt. % to 26%; or 20 wt. %, 20.5 wt. %, 21 wt. %, 21.5 wt. %, 22 wt. %, 22.5 wt. %, 23 wt. %, 23.5 wt. %, 24 wt. %, 24.5 wt. %, 25 wt. %, 25.5 wt. %, 26 wt. %, 26.5 wt. %, 27 wt. %, 27.5 wt. %, 28 wt. %, 28.5 wt. %, 29 wt. %, 29.5 wt. % Crude Fiber 10 wt. % to 20 wt. % of crude fiber on a dry matter basis, or 12 wt. % to 18 wt. %, or 14 wt. % to 16%; or 10 wt. %, 10.5 wt. %, 11 wt. %, 11.5 wt. %, 12 wt. %, 12.5 wt. %, 13 wt. %, 13.5 wt. %, 14 wt. %, 14.5 wt. %, 15 wt. %, 15.5 wt. %, 16 wt. %, 16.5 wt. %, 17 wt. %, 17.5 wt. %, 18 wt. %, 18.5 wt. %, 19 wt. %, 19.5 wt. %.

Feline Food Compositions: Proportion of the composition (% of dry weight of Component composition or parts per million) Leucine and/or a leucine metabolite as described herein, a sirtuin activator as described herein, Protein 30 wt. % to 37 wt. % of crude protein on a dry matter basis or 31 wt. % to 36 wt. %, or 33% to 35%; or 30 wt. %, 30.5 wt. %, 31 wt. %, 31.5 wt. %, 32 wt. %, 32.5 wt. %, 33 wt. %, 33.5 wt. %, 34 wt. %, 34.5 wt. %, 35 wt. %, 35.5 wt. %, 36 wt. %, 36.5 wt. %, 36 wt. % Crude Fat 7.5 wt. % to 9 wt. % of crude fat on a dry matter basis or 7.6 wt. %, 7.7 wt. %, 7.8 wt. %, 7.9 wt. %, 8.0 wt. %, 8.1 wt. %, 8.2 wt. %, 8.3 wt. %, 8.4 wt. %, 8.5 wt. %, 8.6 wt. %, 8.7 wt. %, 8.8 wt. %, 8.9 wt. %, 9.0 Total 30 wt. % to 35 wt. % of total dietary fiber on a dry matter basis Dietary Fiber or 31 wt. % to 34 wt. %, or 32 wt. % to 33%; or 30 wt. %, 30.5 wt. %, 31 wt. %, 31.5 wt. %, 32 wt. %, 32.5 wt. %, 33 wt. %, 33.5 wt. %, 34 wt. %, 34.5 wt. %, 35 wt. % Crude Fiber 20 wt. % to 25 wt. % of crude fiber on a dry matter basis, or 21 wt. % to 24 wt. %, or 22 wt. % to 23%; or 20 wt. %, 20.5 wt. %, 21 wt. %, 21.5 wt. %, 22 wt. %, 22.5 wt. %, 23 wt. %, 23.5 wt. %, 24 wt. %, 24.5 wt. %, 25 wt. %.

Controlled Quantity of Food

In some embodiments, the pet food compositions that include leucine, a leucine metabolite and/or a sirtuin activator can be formulated to provide a complete and balanced daily nutritional food source for a pet in a specified number of pieces. The specified number of pieces can help a pet owner or caretaker in quantifying and administering the proper amount of food to a pet.

In some embodiments, a pet food composition comprises N pieces wherein N pieces provide the complete and balanced daily nutritional requirements of an animal, and N equals 1 to less than 15 pieces, wherein each piece has a caloric content between 50 to 2500 kcal, and wherein N pieces comprise at least about 100 mg of leucine and/or 10 mg of one or more leucine metabolites and at least about 1 mg of a sirtuin activator.

The present invention can be directed to a feeding system for dogs comprising the steps of providing a pet food product in a plurality of sizes wherein each size of the product has a different caloric content; feeding a dog N pieces of the pet food product selected from the plurality of sizes; wherein N equals 1 to 15 in which N pieces provide the complete and balanced daily nutritional requirements of the dog. In the feeding system, N is dependent upon factors selected from a group consisting of an animal's weight, size, breed, life stage, activity level, body condition, health condition and total daily energy requirements.

The pet food of the present invention is formulated to provide the daily nutritional and caloric requirements of an animal. Average required daily caloric intakes for dogs are based on their body weight. Average required daily nutrient intakes are generally based on caloric intake. The serving sizes of food generally vary according to a dog's weight or may be targeted for specific breeds, specific sizes of animals or ages of the animals.

Nutritional requirements are met with three basic products: main meal pet food, snacks and treats. Main meal pet foods are usually sold as complete and balanced foods. Complete and balanced foods mean that when fed to an animal's caloric requirements for the animal's weight, the animal receives all of its required daily nutritional and caloric requirements. Snack foods and treats on the other hand, may not meet these requirements for a number of reasons, such as missing one or more of the required nutrients, not providing the correct level of all of these nutrients and/or not providing enough calories to meet the animal's caloric requirements using the recommended serving size.

Each of the pieces can be formulated to have a specific caloric content which 1) will meet the animal's nutritional requirements within the product's recommended serving size, 2) is designed with a serving size that will provide a satisfying and satiating meal for the animal and 3) is designed with a serving size that the animal can consume in a reasonable amount of time within a day.

Two-Stage Diet

The invention also provides for a diet system for a pet that includes a pet food composition that comprises leucine, a leucine metabolite, and/or a sirtuin activator.

The diet system for promoting comprehensive weight management in companion animals can comprise a first stage pet food composition for promoting weight loss and a second stage pet food composition for maintaining the weight loss, (a) said first stage pet food composition comprising, on a dry matter basis, about 35 to 70% by weight of a protein, about 4 to 10% by weight of a fat, about 2 to 25% by weight of a fiber, about 10 to 35% by weight of a carbohydrate, about 0.05 to 5% by weight of leucine and/or about 0.005 to 1% by weight of one or more leucine metabolites, about 0.0005 to 0.05% by weight of a sirtuin activator, and about 0.1 to 2% by weight of a functional ingredient, wherein said functional ingredient is selected from the group consisting of L-carnitine and conjugated linoleic acid; and (b) said second stage pet food composition comprising, on a dry matter basis, about 20 to 35% by weight of a protein, about 4 to 10% by weight of a fat, about 2 to 25% by weight of a fiber, about 25 to 70% by weight of a carbohydrate, and about 0.1 to 2% by weight of a functional ingredient, wherein said functional ingredient is selected from the group consisting of L-carnitine and conjugated linoleic acid, wherein the protein content of the second stage pet food composition is about 10 to 45% less than the protein content of the first stage pet food composition. Optionally, the amount of leucine, leucine metabolite, and/or sirtuin activator can be any other amount described herein.

In some instances, the invention generally relates to a pet food for use in a comprehensive weight management system for companion animals. More particularly, the weight management system includes two stages: a weight loss stage (stage I) and a weight maintenance stage (stage II). Stage I involves a pet food comprising a high protein, low calorie daily diet that includes a functional ingredient, which further modulates metabolism and builds lean body mass in companion animals. Stage II involves a pet food comprising a low calorie daily diet that maintains the leaner weight of the animal and improves health for the life of the animal.

The process of feeding a companion animal the stage I pet food product of the present invention will promote comprehensive weight management in the companion animal, by promoting weight loss, by increasing the animal's lean body mass, by enhancing the satiety and decreasing voluntary food intake of the animal, by decreasing blood urea nitrogen levels in the animal and reducing the risk of ketosis in the animal. The process of feeding the companion animal the stage II pet food product of the present invention will promote comprehensive weight management in the companion by maintaining the weight loss, the increase in lean body mass, the enhanced satiety, the decreased voluntary food intake, the decreased blood urea nitrogen levels, the reduced risk of ketosis and/or the reduced risk of renal damage of the animal.

Vegetarian

In some embodiments, the subject pet food, treat, and supplement compositions including leucine, a leucine metabolite, and/or a sirtuin activator, can be formulated such that they are vegetarian.

Movement towards more convenient delivery formats for commercial pet foods has seen a great increase in the popularity of dry, packeted pet foods in the form of cereal-based kibbles. Typically these foods are based on cereals such as rice, maize and wheat and are produced by a variety of well-known extrusion techniques.

A drawback for such products is that dogs and cats typically do not find cereal-based products to be as palatable as meat-based products. To counter this, a wide range of technologies has been developed to imbue these cereal-based products with suitably acceptable ‘meat flavors’.

However, it may not always be appropriate to use such meat-based flavorings for packeted pet foods. For example, where such meat-based material are in short supply, or where the marketplace demonstrates a clear demand for genuinely meat-free or vegetarian pet diets. Such demands may stem from perceived health benefits for the animal, or from cultural or religious traditions. Therefore, workers in the field have sought to provide such vegetarian diets, which nevertheless are sufficiently palatable that the animal will readily consume them.

In some embodiments, a vegetarian pet food composition comprises a vegetarian kibble which incorporates a non-meat based flavor-enhancing additive; about 0.05 to 5 wt % of leucine and/or about 0.005 to 1 wt % of one or more leucine metabolites; and about 0.0005 to 0.05 wt % of a sirtuin activator. Optionally, the amount of leucine, leucine metabolite, and/or sirtuin activator can be any other amount described herein.

In other embodiments, a vegetarian pet food or supplement composition comprises a vegetarian kibble which incorporates a non-meat based flavor-enhancing additive; at least about 50 mg of leucine and/or at least about 5 mg of one or more leucine metabolites by weight of the pet food composition; and at least about 1 mg of a sirtuin activator by weight of the pet food composition. Optionally, the amount of leucine, leucine metabolite, and/or sirtuin activator can be any other amount described herein.

The disclosure of the invention provides for a palatable, nutritionally complete vegetarian food for pet mammals, including a nutritionally complete, preferably low moisture, vegetarian kibble which incorporates a non-meat based flavor-enhancing additive. The additive includes a synergistic amount of hydrolyzed vegetable protein and xylose. The combination of these two ingredients provides an especially flavorsome character to the pet food. Typically, the mass ratio of hydrolyzed vegetable protein to xylose, at which this synergistic effect is observed, is between 15:1 and 40:1 on a dry mass basis. The hydrolyzed vegetable protein is available from commercial suppliers. It is typically available as a liquid dispersion, having a solids content of about 15%. Alternatively, it is available as a powder with an approximate moisture content of 10%. It will be apparent to those skilled in the art that if the powdered version is used, commensurate adjustments will need to be made to the level of moisture in the formulation of e.g. the spray to provide the preferred range of hydrolyzed vegetable protein solids in the additive.

Hypoallergenic

In some embodiments, the subject pet food, treat, and supplement compositions including leucine, a leucine metabolite, and/or a sirtuin activator, can be formulated such that they are hypoallergenic.

Food allergies, or food hypersensitivities, commonly afflict household pets such as dogs and cats. These allergies can cause the pet to exhibit symptoms such as excessive itching and scratching, diarrhea or other symptoms which are aggravating both to owner and pet. A housebroken dog with diarrhea who is indoors has a major problem. If the owner is around, the dog pesters the owner to get outside. If the owner is not around the owner may later think the dog is exhibiting undesirable behavior and needs to be punished. Diagnosis is a particular problem for veterinarians, because diagnosis of food hypersensitivity is often difficult and consumes an inordinate amount of veterinarians' time.

Because of the exquisite sensitivity of the immune system to allergens, a pet can be exposed to as few protein sources as possible to avoid the risk of inadvertently exposing the pet to adventitious allergens.

A hypoallergenic pet food composition can comprise proteinaceous component that has been hydrolyzed whereby said component is rendered hypoallergenic to a pet, wherein said proteinaceous component is made up of polypeptides and free amino acids having an average molecular weight of less than about 300, 500, 1000, 1500, 2000, 2500, 3000, 3500, 4000, 4500, 5000, 5500 Daltons; about 0.05 to 5 wt % of leucine and/or about 0.005 to 1 wt % of one or more leucine metabolites; and about 0.0005 to 0.05 wt % of a sirtuin activator. Optionally, the amount of leucine, leucine metabolite, and/or sirtuin activator can be any other amount described herein.

Thus, a hypoallergenic pet food can comprise a composition comprising a hydrolysate in an acceptable, semi-solid formulation. In some configurations, a hydrolysate comprised by a hypoallergenic pet food described herein can be a frozen hydrolysate, a freshly prepared hydrolysate or a hydrolysate that is stored refrigerated before use. Furthermore, a hydrolysate comprised by a hypoallergenic pet food described in various configurations herein can be a hydrolysate prepared by a method comprising freezing the hydrolysate, vacuum drying the hydrolysate, spray drying the hydrolysate, drum drying the hydrolysate or freeze drying the hydrolysate. In some configurations, the hydrolysate can be prepared by a method which comprises freezing the hydrolysate.

Customized Diets

In some embodiments, the subject pet food, treat, and supplement compositions can be tailored to a subject pet by a pet owner or caretaker. The pet caretaker can specify characteristics of the pet, such as weight, height, age, and breed which are then used to create a customized diet, including a pet food, treat, or supplement, that incorporates leucine, a leucine metabolite, and/or a sirtuin activator.

In some embodiments, the subject pet food, treat, and supplement compositions can be tailored to a subject pet based on the pet's indication of its preferences. The pet can choose among an array of choices that allow a pet owner to interpret the pet's preferences, which can then in turn be used to create a customized diet, including a pet food, treat, or supplement, that incorporates leucine, a leucine metabolite, and/or a sirtuin activator.

One aspect of the present invention provides a method for determining the preferred macronutrient content of a diet for an individual animal, the method comprising: providing to said animal food compositions which provide an enriched source of fat, protein and/or carbohydrate, such that said animal can select and consume preferred quantities of said food compositions in order to achieve an preferred consumption of fat, protein and carbohydrate; allowing said animal to consume preferred quantities of fat, protein and carbohydrate from said compositions; and determining, from the consumed amount of fat, protein and carbohydrate from said compositions, the preferred macronutrient content of a diet for said individual animal.

In some embodiments, a method for feeding a pet can comprise providing, over an extended and preselected period of time, different food compositions to said animal in which each composition provides an enriched source of fat, protein or carbohydrate, such that said animal can select and consume different and preferred quantities of each said food compositions in order to achieve an preferred consumption of fat, protein and carbohydrate for said animal; wherein at least one of said food compositions comprises about 0.05 to 5 wt % of leucine and/or about 0.005 to 1 wt % of one or more leucine metabolites; and about 0.0005 to 0.05 wt % of a sirtuin activator; allowing said animal to consume the different and preferred quantities of fat, protein and carbohydrate from each of said compositions over the extended preselected period of time; and determining, from the consumed amount of fat, protein and carbohydrate from each of said compositions, a customized dietary regime that provides the preferred macronutrient content of a diet for said individual animal. Optionally, the amount of leucine, leucine metabolite, and/or sirtuin activator can be any other amount described herein.

In order for the animal to select the preferred macronutrient content from unlimited amounts of said compositions, the fat, protein and carbohydrate must be provided in a format or formats such that the animal can select preferred quantities thereof. Accordingly, the sources of fat, protein and carbohydrate can be provided in two or more different compositions, each composition having differing levels of at least protein and fat.

The method can comprise a learning phase. During such a learning phase, the animal can be provided with a single diet composition at any one feeding experience. Accordingly, the animal has no other choice at that feeding experience. Each diet composition comprises a foodstuff which is enriched in respect of one macronutrient. The length of the learning phase depends on a number of factors, including how many feeding experiences the animal has during a day/week etc and how much length of time is desired or available for the animal to learn.

A helpful tool may be a table or chart which indicates a preferred diet or foodstuff depending on the quantities of each of the compositions consumed by the animal. Alternatively, the quantities of each composition consumed may relate to a figure or number which can be used to obtain an preferred diet by use, for example, of a vending machine system. Such a system can dispense an preferred diet either as a single dietary foodstuff, or as two or more components which are to be available to the animal at the same time.

In some embodiments, one or more steps for providing a food composition for a pet can include selecting or customizing the pet food based on the pet's characteristics or preferences. The invention provides for a method of producing a customized dry pet food product formulated from a dry pet food kibble recipe and selected functional ingredients, the formulation being selected on the basis of an individual pet's attributes and physical conditions, the method comprising: providing a plurality of different formulations of pre-made dry kibble pieces; selecting a predetermined volume of dry kibble pieces from the plurality of different formulations of pre-made dry kibble pieces; providing a plurality of functional ingredients; coating the volume of dry kibble pieces with one or more of the plurality of functional ingredients; and packaging and labeling the predetermined volume of coated dry kibble pieces; wherein the selection of the predetermined volume of dry kibble pieces and the one or more functional ingredients is based on the individual pet's attributes and physical conditions to provide the customized dry pet food product, and wherein the coating and/or the kibble pieces comprise leucine and/or a leucine metabolite and a sirtuin activator.

In some embodiments, one or more steps for providing a food composition for a pet can include selecting or customizing the pet food based on the pet's characteristics or preferences using the aid of a computer system.

The invention provides for a computer-readable medium comprising code that, upon execution by one or more processors, implements a method of producing a customized dry pet food composition formulated from a dry pet food kibble recipe and selected functional ingredients, the formulation being selected on the basis of an individual pet's attributes and physical conditions, the method comprising: a. receiving information on the individual pet's attributes and physical conditions; selecting a predetermined volume of dry kibble pieces from a plurality different formulations of pre-made dry kibble pieces; selecting one or more functional ingredients from a plurality functional ingredients; coating the predetermined volume of dry kibble pieces with the one or more functional ingredients; and packaging and labeling the predetermined volume of coated dry kibble pieces; wherein the selection of the predetermined volume of dry kibble pieces and one or more functional ingredients is based on the individual pet's attributes and physical conditions to provide the customized dry pet food product, and wherein the coating and/or the kibble pieces comprise leucine and/or a leucine metabolite and a sirtuin activator.

One or more steps of methods described herein may be implemented in hardware. Alternatively, one or more steps may be implemented in software stored in, for example, one or more memories or other computer readable medium and implemented on one or more processors. As is known, the processors may be associated with one or more controllers, calculation units, and/or other units of a computer system, or implanted in firmware as desired. If implemented in software, the routines may be stored in any computer readable memory such as in RAM, ROM, flash memory, a magnetic disk, a laser disk, or other storage medium, as is also known. Likewise, this software may be delivered to a computing device via any known delivery method including, for example, over a communication channel such as a telephone line, the internet, a wireless connection, etc., or via a transportable medium, such as a computer readable disk, flash drive, etc. The various steps may be implemented as various blocks, operations, tools, modules and techniques which, in turn, may be implemented in hardware, firmware, software, or any combination of hardware, firmware, and/or software. When implemented in hardware, some or all of the blocks, operations, techniques, etc. may be implemented in, for example, a custom integrated circuit (IC), an application specific integrated circuit (ASIC), a field programmable logic array (FPGA), a programmable logic array (PLA), etc. A computer system may be involved in one or more of sample collection, sample processing, genotyping, data analysis, calculation of weighted risks, calculation of aggregated risk score, comparison of aggregated risk score to a threshold score, determination of a subject's absolute or increased risk, generating a report, and reporting results to a receiver.

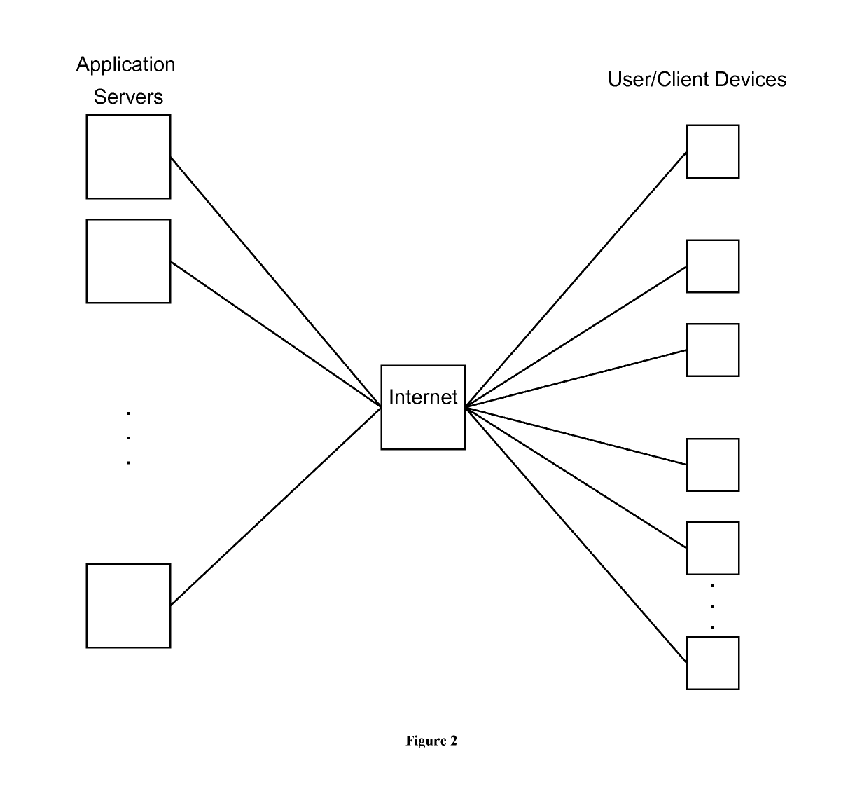

A client-server, as shown in FIG. 2, relational architecture can be used in embodiments of the invention. A client-server architecture is a network architecture in which each computer or process on the network is either a client or a server. Server computers are typically powerful computers dedicated to managing disk drives (file servers), printers (print servers), or network traffic (network servers). Client computers include PCs (personal computers) or workstations on which users run applications, as well as example output devices as disclosed herein. Client computers rely on server computers for resources, such as files, devices, and even processing power. In some embodiments of the invention, the server computer handles all of the database functionality. The client computer can have software that handles all the front-end data management and can also receive data input from users.

As shown in FIG. 2, a user/client device, such as a computer system, can be connected to an analysis system by a network connection. The computer system may be understood as a logical apparatus that can read instructions from media and/or a network port, which can optionally be connected to server having fixed media. The system can include a CPU, disk drives, optional input devices such as keyboard and/or mouse, and optional monitor. Data communication can be achieved through the indicated communication medium to a server at a local or a remote location. The communication medium can include any means of transmitting and/or receiving data. For example, the communication medium can be a network connection, a wireless connection, or an internet connection. Such a connection can provide for communication over the World Wide Web. In some embodiments, a physical report is generated and delivered to a receiver.

In some embodiments there is provided a computer readable medium encoded with computer executable software that includes instructions for a computer to execute functions associated with the identified one or more alleles and/or genotypes. Such computer system may include any combination of such codes or computer executable software, depending upon the types of evaluations desired to be completed. The system can have code for that, upon execution by one or more processors, implements a method of producing a customized pet food, such as a dry pet food, composition formulated from a pet recipe, such as a dry pet food kibble recipe, and selected functional ingredients, the formulation being selected on the basis of an individual pet's attributes and physical conditions, the method comprising receiving information on the individual pet's attributes and physical conditions; selecting a predetermined volume of food, such as dry kibble pieces, from a plurality different formulations of pre-made food, such as dry kibble pieces; selecting one or more functional ingredients from a plurality functional ingredients; coating the predetermined volume of food, such as dry kibble pieces, with the one or more functional ingredients; and packaging and labeling the predetermined volume of coated food, such as dry kibble pieces. The food can be packaged by a manufacturer or by a retailer of pet foods. The packaging can be fully automated, such that human intervention is not required by the manufacturer to package the food, or is minimized.

The selection of the predetermined volume of food, such as dry kibble pieces, and one or more functional ingredients is based on the individual pet's attributes and physical conditions to provide the customized pet food product, which may be dry. Additionally or alternatively, the customized pet food can be based on the pet's preferences, as indicated by the pet's choice of food when presented with an array of options, as described herein. Also, the coating and/or the kibble pieces comprise leucine and/or a leucine metabolite and a sirtuin activator.

The system can also have code for generating a report. The report can include one or more recommendations for a customized pet diet that is based on the characteristics or preferences of the pet.

After performing a calculation, a processor can provide the output, such as from a calculation, back to, for example, the input device or storage unit, to another storage unit of the same or different computer system, or to an output device. Output from the processor can be displayed by data display. A data display can be a display screen (for example, a monitor or a screen on a digital device), a print-out, a data signal (for example, a packet), an alarm (for example, a flashing light or a sound), a graphical user interface (for example, a webpage), or a combination of any of the above. In an embodiment, an output is transmitted over a network (for example, a wireless network) to an output device. The output device can be used by a user to receive the output from the data-processing computer system. After an output has been received by a user, the user can determine a course of action, or can carry out a course of action, such as a medical treatment when the user is medical personnel. In some embodiments, an output device is the same device as the input device. Example output devices include, but are not limited to, a telephone, a wireless telephone, a mobile phone, a PDA, a flash memory drive, a light source, a sound generator, a fax machine, a computer, a computer monitor, a printer, an iPod, and a webpage. The user station may be in communication with a printer or a display monitor to output the information processed by the server.

It is envisioned that data relating to the present disclosure can be transmitted over a network or connections for reception and/or review by a receiver. The receiver can be but is not limited to an individual; the subject to whom the report pertains; a health care provider, manager, other healthcare professional, or other caretaker; a genetic counselor; a person or entity that performed and/or ordered the genotyping analysis; or a local or remote system for storing such reports (e.g. servers or other systems of a “cloud computing” architecture).

Sterilization

Additional embodiments of the present invention include a method of making a pet food, supplement, or treat including at least one heat treating step for microbe deactivation (kill), such as salmonella, and compositions and kits that include pet foods, treats, or supplements that are substantially or essentially free of microbes. The pet food can be in any form of the embodiments described herein. In one embodiment, a non-limiting example of which is a coated kibble that comprises a core and a coating as hereinabove described, two heat treating deactivation steps can be performed. The core can be formed through extruding, as described hereinabove. After extruding into a core, the core can be heat treated in a manner to sufficiently deactivate any salmonella present in the core. Subsequently, prior to, or contemporaneously, the coating can be formed and heat treated in a similar manner as that of the core to deactivate any salmonella present. The coated kibble can then be formed, as described hereinabove, by coating the core with the coating.

Salmonella deactivation generally requires the application of heat while the microbes are in a moist environment. Once completely dry, salmonella can become dormant and resist efforts using dry heat to deactivate them. In a moist environment, salmonella are more readily deactivated. For example, the application of heat at 80° C. for greater than about two minutes can effectively deactivate salmonella when in a moist environment. Application of temperatures higher than 80° C. in moist environments results in correspondingly shorter times needed to deactivate the salmonella.

Superheated steam has been used effectively in many industries to deactivate salmonella. Superheated steam is defined as steam at a temperature greater than the boiling point of water for the existing pressure. Most industrial use of superheated steam utilizes pure or substantially pure steam. The non-steam component is usually air.

In one embodiment the salmonella deactivation step may be performed in a vibrating conveyor, such as a spiral elevator, as disclosed herein. In one embodiment, steam can be injected into the vibrating conveyor for microbe control. Steam can be injected into the coils of the vibrating conveyor at any point, and even through multiple points. Such injection can be through ports as disclosed herein.

In one embodiment, steam can be injected into the pipe on one side of a coil of the conveyor and can be extracted from the pipe at the other side of the coil by an exhaust manifold attached to the coil. In one embodiment, the channel, such as a pipe, can be heated to greater than about 100° C., or greater than about 110° C., or greater than about 125° C. to prevent condensation of steam inside the pipe. In one embodiment, the channel can be heated in sections. In another embodiment, the pipe can be heated entirely. In other embodiments, jackets of steam or water can be used for heating, electrical tape can be used for heating, and current can be run through the pipe itself for heating.

In an embodiment wherein steam is injected, once inside the pipe, the steam contacts and treats all of the fluidized kibbles such that they are substantially free of microbes, such as salmonella.

Compartmentalized Packaging

In some embodiments of the invention, the pet foods, treats, or supplements can be packaged such that two different formulations are compartmentalized within the package. The compartmentalization of the two different formulations can allow for a pet owner to provide an array of choices for the pet. The two different formulations can differ in one or more components, such as fat, protein, or carbohydrate.

A multi-component pet food composition can comprise two or more compartmentalized food compositions, wherein the at least two compartmentalized compositions differ in their content in at least two of fat, protein or carbohydrate, and further wherein one of the two or more compartmentalized compositions comprises about 0.05 to 5 wt % of leucine and/or about 0.005 to 1 wt % of one or more leucine metabolites, and about 0.0005 to 0.05 wt % of a sirtuin activator. Optionally, the amount of leucine, leucine metabolite, and/or sirtuin activator can be any other amount described herein.

The present invention provides a canine or feline multi-component foodstuff comprising two or more compartmentalized food compositions of which at least two of the compositions differ in their content of at least two selected from the group consisting of fat, protein and carbohydrate.

By the term compartmentalized it is meant that the two or more food compositions are not mixed. They may be provided on or in different containers, such as a bowl, plate, packaging. The containers may or may not be sealed. The multi-component meal comprising the two or more food compositions may be provided in unlimited quantities to the feline animal.

The compositions encompass any product that a pet consumes in its diet. Thus, the compositions may include the standard food products as well as pet food snacks (for example snack bars, cereal bars, snacks, treats, biscuits and sweet products). The composition may be a cooked product. It may incorporate meat or animal-derived material (such as beef, chicken, turkey, lamb, fish, blood plasma, marrowbone, etc or one or more thereof). Alternatively the composition may be meat-free (preferably including a meat substitute such as soya, maize gluten or a soya product) in order to provide protein. The composition may contain additional protein sources such as soya protein concentrate, milk, protein, gluten, etc. The composition may also contain starch, such as one or more grains (e.g. wheat, corn, rice, oats, barley, etc) or may be starch-free. The composition may incorporate or be a gelatinized starch matrix. The composition may incorporate one or more types of fiber such as sugar beet pulp, chicory pulp, chicory, coconut endosperm fiber, wheat fiber etc. Dairy products, such as those incorporating a cream or a cheese sauce, may be suitable. The composition can also be newly designed products currently not available. The most suitable composition may be a pet food product as described herein which is sold as a pet food, in particular a pet food for a domestic dog or a domestic cat. It may be convenient to provide the compositions in a dry format, such as dried ready-to-eat cereal products (often referred to as kibbles).

Multi-Formulation Daily Diet

In other embodiments, the subject pet food, treat, and supplement compositions that include leucine, a leucine metabolite, and/or a sirtuin activator can be utilized in diet systems that include the feeding of a variety of formulations each day.

In some embodiments, a diet for companion animals can comprise a first stage pet food composition and a second stage pet food composition for maintaining the weight loss, wherein each of said first stage pet food composition and said second stage pet food composition comprise, on a dry matter basis, about 0.05 to 5 wt % of leucine and/or about 0.005 to 1 wt % of one or more leucine metabolites, and about 0.0005 to 0.05 wt % of a sirtuin activator, and wherein said second stage pet food comprises at least about 5% higher fat content compared to said first stage pet food. Optionally, the amount of leucine, leucine metabolite, and/or sirtuin activator can be any other amount described herein.

In accordance with typical feeding patterns for companion animals, food products can be provided as meals in the morning and in the afternoon or evening. Additional food products may be provided in between, such as mid-morning, during the middle part of the day, mid-afternoon, or in the evening. Companion animals have, in accordance with the findings of the present invention, shown preferences for macronutrient content for such particular events. In addition, caregiver/owners of companion animals are able to easily identify suitable food products by their labeling for administration for a particular event.

The companion animals of the present invention can be, in particular, the domestic cat (Felis domesticus) or the domestic dog (Canis domesticus). Other companion animals include fish, birds and horses.

The dietary regime of the present invention can comprise one pet food product for feeding as the morning meal and one pet food product for feeding as the afternoon/evening meal. Additional snacks for in between meals or additions to the main meal (e.g. kibbles) may be included.

The invention includes any dietary regime or sequence of products fed at prescribed times or in a prescribed order that either accommodate a physiological need or modify a physiological response and/or are designed for administration for a particular event and may include: A breakfast food for administration as the first food of the day, containing an energy content and a macronutrient profile appropriate to the level of expected activity in the day, a dinner or supper food for administration as the last food of the day, with an energy content and macronutrient profile appropriate to a inactive or sleeping animal, a fiber-controlled diet for avoiding toileting during the night, and food for particular seasons, for example designed in relation to the nutritional needs of the skin and coat.

One macronutrient preference which has been shown by companion animals is for an increase in the total fat content of the afternoon/evening meal compared to the morning meal. Accordingly, the dietary regime of the invention can include one of the pet food products having a higher content of fat than one other pet food product in a daily regime. The product with the higher fat content can be fed to the companion animal as the afternoon/evening meal.

A preferred feature of the present invention results from a demonstrated increase in relative intake for the higher fat product in the afternoon/evening meal compared with products with a lower fat content. The higher fat content can be in replacement of protein or in replacement of carbohydrate.

Further preferences for the food of the dietary regime of the present invention are that the fat content of the afternoon/evening food can be higher than the fat content of the morning food by at least 5% of the total calorie content of the food. Further preferences are that the calories contributed by the fat content of the food for the morning can be between 20% and 70% of the total calorie content of the food and the fat content of the food for the afternoon/evening can contribute between 25% and 75% of the total calorie content of the food. The food fed in the afternoon/evening can be higher in fat content than the morning food by at least 5% of the total calorific value of the product, by 10%, or by 15%.

The pet food products as part of the dietary regime according to the present invention encompass any product that a pet consumes in its diet. Thus, the invention covers the standard food products as well as pet food snacks (for example snack bars, cereal bars, snacks, biscuits and sweet products). The food product may be a cooked product. It may incorporate meat or animal-derived material (such as beef, chicken, turkey, lamb, fish, blood plasma, marrowbone, etc or one or more thereof). The product alternative may be meat-free (preferably including a meat substitute such as soya, maize gluten or a soya product) in order to provide a protein source. The product may contain additional protein sources such as soya protein concentrate, milk, protein, gluten, etc. The product may also contain a starch source such as one or more grains (e.g. wheat, corn, rice, oats, barley, etc) or may be starch-free. The product may incorporate or be a gelatinized starch matrix. The product may incorporate one or more types of fiber such as sugar beet pulp, chicory pulp, chicory, coconut endosperm fiber, wheat fiber etc. The content of the product/ingredients contributes towards the macronutrient profile of the food. Thus, food products which according to the present invention provide a higher fat content will be designed accordingly. Dairy products, such as those incorporating a cream or a cheese sauce, may be suitable. The present invention is particularly relevant for a pet food product as described herein which is sold as a pet food, in particular a pet food for a dog or a cat.

Diabetes

Diabetes mellitus is a disease characterized by hyperglycemia; altered metabolism of lipids, carbohydrates and proteins; an increased risk of complications from vascular disease; inflammation; and insulin sensitivity. Diabetes is an increasing pet health problem, as it is associated with both increasing age and obesity.

There are two major types of diabetes mellitus: 1) Type I, also known as insulin dependent diabetes (IDDM) and 2) Type II, also, known as insulin independent or non-insulin dependent diabetes (NIDDM). Both types of diabetes mellitus are due to insufficient amounts of circulating insulin and a decrease in the response of peripheral tissue to insulin.

The early symptoms of untreated diabetes mellitus are related to elevated blood sugar levels, and loss of glucose in the urine. High amounts of glucose in the urine can cause increased urine output and lead to dehydration. Dehydration causes increased thirst and water consumption. The inability to utilize glucose energy eventually leads to weight loss despite an increase in appetite. Some untreated diabetes patients also complain of fatigue, nausea, and vomiting. Pets with diabetes are prone to developing infections of the bladder, skin, and vaginal areas. Fluctuations in blood glucose levels can lead to blurred vision. Extremely elevated glucose levels can lead to lethargy and coma (diabetic coma). Diabetes can occur in many animals and breeds, especially dogs and cats.

In inflammatory diseases, such as rheumatoid arthritis, pathologic inflammatory processes can lead to morbidity and mortality. The cytokine tumor necrosis factor-alpha (TNF-alpha) plays a central role in the inflammatory response and has been targeted as a point of intervention in inflammatory disease. TNF-alpha is a polypeptide hormone released by activated macrophages and other cells. At low concentrations, TNF-alpha participates in the protective inflammatory response by activating leukocytes and promoting their migration to extravascular sites of inflammation (Moser et al., J Clin Invest, 83:444-55, 1989). At higher concentrations, TNF-alpha can act as a potent pyrogen and induce the production of other pro-inflammatory cytokines (Haworth et al., Eur J Immunol, 21:2575-79, 1991; Brennan et al., Lancet, 2:244-7, 1989). TNF-alpha also stimulates the synthesis of acute-phase proteins. In rheumatoid arthritis, a chronic and progressive inflammatory disease affecting about 1% of the adult U.S. population, TNF-alpha mediates the cytokine cascade that leads to joint damage and destruction (Arend et al., Arthritis Rheum, 38:151-60, 1995).

Interleukin-6 (IL-6) is another pro-inflammatory cytokine that exhibits pleiotropy and redundancy of action. IL-6 participates in the immune response, inflammation and hematopoiesis. It is a potent inducer of the hepatic acute phase response and is a powerful stimulator of the hypothalamic-pituitary-adrenal axis that is under negative control by glucocorticoids. IL-6 promotes the secretion of growth hormone but inhibits release of thyroid stimulating hormone. Elevated levels of IL-6 are seen in several inflammatory diseases, and inhibition of the IL-6 cytokine subfamily has been suggested as a strategy to improve therapy for rheumatoid arthritis (Carroll et al., Inflamm Res, 47:1-7, 1998). In addition, IL-6 has been implicated in the progression of atherosclerosis and the pathogenesis of coronary heart disease (Yudkin et al., Atherosclerosis, 148:209-14, 1999).

The cytokine IL-1 beta is another protein involved in the inflammatory response. It stimulates thymocyte proliferation, fibroblast growth factor activity, and the release of prostaglandin from synovial cells.

Irisin is a protein believed to replicate the weight loss effects of exercise. Irisin may be beneficial in decreasing and/or preventing the incidence of diabetes in pets.

Metformin is a compound derived from biguanides that primarily acts by reducing hepatic gluconeogenesis, but also reduces glucose absorption at the gastro-intestinal tract level and increases sensitivity to insulin by increasing the peripheral utilisation of glucose. This may be due to the fact that metformin improves the binding of insulin to its cellular receptor, which is explained by the increased activity that it induces in the tyrosine kinase postreceptor and the consequent increase in the number and activity of GLUT4 carriers. Metformin is not metabolised; it is directly excreted in the urine. Its half-life is 6.2 hours.

A subject composition, which may be a pet food, treat, snack, supplement, or drink, can comprise a metformin content of at least about 0.025 g/kg, 0.05 g/kg, 0.075 g/kg, 0.1 g/kg, 0.15 g/kg, 0.25 g/kg, 0.5 g/kg, 0.75 g/kg, 1 g/kg, 1.5 g/kg, 2 g/kg, 2.5 g/kg, 3 g/kg of the diet comprising the subject composition. In some instances the composition comprises a metformin content of about 1.5 g/kg of the diet comprising the subject composition. In some instances the composition comprises a metformin content of about 0.75/kg of the diet comprising the subject composition. In some instances the composition comprises a metformin content of about 0.25 g/kg of the diet comprising the subject composition.

A subject composition, which may be a pet food, treat, snack, supplement, or drink, can comprise a metformin content of at least about 12.5, 25, 50, 75, 100, 125, 150, 175, 200, 225, 250 mg of the diet comprising the subject composition. In some instances the composition comprises a metformin content of about 125 mg of the diet comprising the subject composition.

The invention provides for a method of increasing irisin production, comprising administering to the subject any of the compositions described herein, wherein irisin production in the subject increases over a time period. In some embodiments, the increase in irisin production (or in an indicator providing evidence thereof) is an increase of about, or more than about 5%, 10%, 20%, 30%, 40%, 50%, 60%, 70%, 80%, 90%, 100%, 125%, 150%, 175%, 200%, or more. In some embodiments, the increase in irisin production (or in an indicator providing evidence thereof) is an increase of about, or more than about 1-fold, 3-fold, 5-fold, 6-fold, 8-fold, 10-fold, 15-fold, 20-fold, 50-fold, or more. In some embodiments, the increase in irisin production is evidenced by an increase in FNDC5 expression (e.g. as measured from mRNA and/or protein level).

The invention provides for a method of treating diabetes, comprising administering to the subject any of the compositions described herein over a time period, wherein the insulin sensitivity in the subject is increased over the time period. Insulin sensitivity can be increased by about or greater than about 1, 2, 3, 5, 10, 20, 50, 100, or 200%. In some embodiments, a branched chain amino acid (or a metabolite thereof) and/or a sirtuin pathway activator are administered in an amount that reduces the therapeutically effective dose of metformin for a subject. In some embodiments, the therapeutically effective dose of metformin is reduced by about or more than about 50%, 60%, 70%, 80%, 90%, 95%, 97.5%, 99.9%, 99.99%, or more. In some embodiments, administration of compositions of the invention reduces body fat (e.g. visceral fat) by about or more than about 5%, 10%, 15%, 20%, 25%, 50%, or more.

Insulin sensitivity can be measured using a variety of techniques, including HOMA_{IR}. HOMA_{IR}, which is the homeostasis model assessment of insulin resistance can be used as a screening index of changes in insulin sensitivity. HOMA_{IR} can be calculated via standard formula from fasting plasma insulin and glucose as follows: HOMA_{IR}=[Insulin (uU/mL)×glucose (mM)]/22.5.

In some embodiments, insulin signaling can also be measured. Insulin signaling can be measured by measuring total and phosphorylated Akt, GSK-3β, IGF-1R, IR, IRS-1, p70S6K and PRAS40 in tissue lysates via the Luminex Kits “Akt Pathway Total 7-Plex Panel” (Cat# LHO0002) and “Akt Pathway Phospho 7-Plex Panel” (Cat# LHO0001) from Invitrogen Life Science.

Methods of Use

The invention provides for methods of regulating energy metabolism in a pet by administering one or more subject compositions. These compositions include the combination compositions described herein, such as combination compositions comprising leucine, a leucine metabolite, and/or a sirtuin activator. The combination compositions can be formulated as a pet food, pet treat, pet supplement or a pet drink.

The methods compositions can be comprise administering to a pet an effective amount of (a) leucine and/or one or more metabolites thereof, and (b) a sirtuin activator, wherein the combination when administered to a subject in need thereof enhances energy metabolism, including cellular metabolism, and mitochondrial biogenesis. The composition, when administered to a subject in need thereof, can enhance energy metabolism, including cellular metabolism and mitochondrial biogenesis, as measured by a decrease in weight gain of a subject, a decrease in adipose volume of a subject, an increase in fat oxidation of a subject, an increase in insulin sensitivity of a subject, a decrease in oxidative stress markers of a subject, and/or a decrease in inflammatory markers of a subject. In some embodiments, the composition is substantially free of free or individual non-branched chain amino acids or non-leucine amino acids.

The enhanced energy metabolism can be quantified by an increase in weight loss of a subject by at least 5, 10, 30, or 40%, a decrease in weight of about 1, 2, or 3 kg, a decrease in body condition score of at least about 1, 2 or 3, an increase in fat loss of a subject by at least about 1, 5, 10, 20, 30, or 50%, or an increase in insulin sensitivity by at least about 1, 5, 10, or 15% when the composition is administered to the subject. The enhanced energy metabolism can be measured relative to the dosing of the subject with a placebo, or relative to the subject prior to administration of the subject composition.

The compositions can be administered to a subject orally or by any other methods. Methods of oral administration include administering the composition as a liquid, a solid, or a semi-solid that can be taken in the form of a dietary supplement or a food stuff

The compositions can be administered periodically. For example, the compositions can be administered one, two, three, four times a day, or even more frequent. The subject can be administered or fed the subject compositions every 1, 2, 3, 4, 5, 6 or 7 days. In some embodiments, the compositions are administered once, twice, or three times daily. The administration can be concurrent with meal time of a subject. The period of treatment or diet supplementation can be for about 1, 2, 3, 4, 5, 6, 7, 8, or 9 days, 2 weeks, 1-11 months, or 1 year, 2 years, 5 years or even longer. In some embodiments of the invention, the dosages that are administered to a subject can change or remain constant over the period of treatment. For example, the daily dosing amounts can increase or decrease over the period of administration.

The compositions can be administered to a subject such that the subject is administered a selected total daily dose of the composition. The total daily dose can be determined by the sum of doses administered over a 24 hour period. The total daily dose of the composition can include at least about 10, 50, 100, 150, 200, 250, 500, 750, 1000, 1125, 2000, 2250 mg or more of a leucine or metabolite thereof, such as HMB. The total daily dose of the composition can include at least about 1, 3, 7.5, 15, 30, 45, 90 mg or more of a sirtuin activator. The total daily dose of the composition can have a mass ratio of leucine or metabolite thereof to a sirtuin activator that is about, greater than about, or less than about 10, 20, 30, 45, 50, 55, 60, 65, 70, 75, 80, 85, 90, 100, 110, 120, 130, 140, 150, 175, 200, 250, 500, 750, 1000, or more.

The invention also provides for administering to a pet an effective amount of leucine, a leucine metabolite, and/or a sirtuin activator that is effective in inducing a selected circulating level in the pet of leucine, the leucine metabolite and/or a sirtuin activator. The subject compositions can induce a circulating level that is: about or greater than about 0.25, 0.5, 0.75, or 1 mM of leucine or leucine metabolite and/or about or greater than about 10, 25, 50, 100, 150, or 200 nM a sirtuin activator.

Kits

The invention also provides kits. The kits include one or more compositions described herein, in suitable packaging, and may further comprise written material that can include instructions for use, discussion of clinical studies, listing of side effects, and the like. Such kits may also include information, such as scientific literature references, package insert materials, clinical trial results, and/or summaries of these and the like, which indicate or establish the activities and/or advantages of the composition, and/or which describe dosing, administration, side effects, drug interactions, or other information useful to the health care provider. Such information may be based on the results of various studies, for example, studies using experimental animals involving in vivo models and studies based on human clinical trials. A kit may comprise one or more unit doses described herein. In some embodiments, a kit comprises about, less than about, or more than about 1, 2, 3, 4, 5, 6, 7, 8, 9, 10, 11, 12, 13, 14, 15, 16, 17, 18, 19, 20, 30, 31, 60, 90, 120, 150, 180, 210, 365, or more days of supply. Instructions for use can comprise administration instructions, such as instructions to take 1, 2, 3, 4, 5, 6, 7, 8, 9, 10, or more units 1, 2, 3, 4, 5, 6, 7, 8, 9, 10, or more times per day. For example, a kit may comprise a unit supplied as a tablet, with each tablet package separately, multiples of tablets packaged separately according to the number of units per administration (e.g. pairs of tablets), or all tablets packaged together (e.g. in a bottle). As a further example, a kit may comprise a unit supplied as a bottled drink, the kit comprising 1, 2, 3, 4, 5, 6, 7, 8, 9, 10., 11, 12, 13, 14, 24, 28, 36, 48, 72, or more bottles.

The kit may further contain another agent. In some embodiments, the compound of the present invention and the agent are provided as separate compositions in separate containers within the kit. In some embodiments, the compound of the present invention and the agent are provided as a single composition within a container in the kit. Suitable packaging and additional articles for use (e.g., measuring cup for liquid preparations, foil wrapping to minimize exposure to air, and the like) are known in the art and may be included in the kit. Kits described herein can be provided, marketed and/or promoted to health providers, including physicians, nurses, pharmacists, formulary officials, and the like. Kits may also, in some embodiments, be marketed directly to the consumer.

In some embodiments, a kit can comprise a multi-day supply of unit dosages. The unit dosages can be any unit dosage described herein. The kit can comprise instructions directing the administration of the multi-day supply of unit dosages over a period of multiple days. The multi-day supply can be a one-month supply, a 30-day supply, or a multi-week supply. The multi-day supply can be a 90-day, 180-day, 3-month or 6-month supply. The kit can include packaged daily unit dosages, such as packages of 1, 2, 3, 4, or 5 unit dosages. The kit can be packaged with other dietary supplements, vitamins, and meal replacement bars, mixes, and beverages.

In another embodiment, the present invention provides kits suitable for improving stool quality and/or stool frequency for an animal. Said kits may comprise combinations of cations, anions, foods, other compounds, agents or medicaments, and instructions for using said kit components for improving stool quality and/or stool frequency for an animal in need thereof. For example, it is contemplated that kits of the present invention may comprise metabolizable cations chosen from calcium, sodium, potassium, magnesium, and mixtures thereof; metabolizable anions may be chosen from phosphorus, chloride, sulfur, and mixtures thereof. Any and all forms of said metabolizable cations and anions are contemplated, including pharmaceutically acceptable salt forms. The components of the kits may further comprise a gastrointestinal tract-improving agent, an anti-diarrhea agent and/or an anti-constipation agents and instructions for use thereof.

For example, a kit may comprise a nutritionally complete food, e.g., a puppy food in addition to a metabolizable cation, and/or a metabolizable anion with instructions regarding how to increase the DCAB of the food composition with the metabolizable cation and/or instructions as to how to decrease the DCAB of the food composition with metabolizable anions in order to achieve a desired improvement in stool quality in an animal in need thereof. In some embodiments, the kit may further comprises one or more anti-diarrhea agents, anti-constipation agents, and/or gastrointestinal tract-improving agents and instructions for use thereof with the food and metabolizable cations and anions. It is understood that addition of cations will cause the stool to be more firm; the addition of anions will cause the stool to be less firm. Based on the teachings of the present invention, one of skill in the art will understand how to modify the DCAB in the animal depending on the condition of the stool of the animal to be treated and the change in stool firmness desired.

### EXAMPLES

### Example 1

### Leucine+Sirt Activator Supplemented Diet Effect on Percentage Insulin Sensitivity and Fat Oxidation

To assess the efficacy of the subject compounds on weight and fat loss in obese dogs, obese dogs are administered a leucine+a sirt activator supplemented diet. Specifically, the objective is to determine the effect of leucine+a sirt activator supplemented diet on the percentage body fat, body weight, and body condition scores of beagles as compared to a placebo and a prescription diet (RD Diet). The leucine+a sirt activator supplemented diet comprised 1 g leucine+50 mg resveratrol/day.

Using experimental models of obesity in dogs, dogs administered a leucine+sirt activator supplemented diet are expected to exhibit increases in insulin sensitivity and fat oxidation when compared with placebo.

Healthy adult beagles (n=18, 9 males and 9 females) are evaluated for four weeks to establish individual caloric requirements. Obesity is induced in the dogs by feeding them a hypercaloric diet by adding 2 lbs of fat with some meat daily in order to induce obesity defined as a body condition score of 8 or 9 out of 9 and having a body fat content of at least 35-40% using dual energy x-ray absorptiometry (Mawby D I, Bartges J W, d'Avignon A, et al. Comparison of various methods for estimating body fat in dogs. J Am Anim Hosp Assoc 2004; 40:109-114. Ionut V, Liu H, Mooradian V, et al. Novel canine models of obese prediabetes and mild type 2 diabetes. Am J Physiol Endocrinol Metab 2010; 298:E38-48. Herein incorporated by reference).

After achieving a body fat content of 35-40%, dogs are randomly assigned to 1 of 3 groups so that there are 3 males and 3 females per each group: Group 1 is fed the maintenance diet at ideal body weight and received a leucine+a sirt activator supplemented diet, Group 2 is fed the maintenance diet at ideal body weight and receives the placebo, and Group 3 dogs are fed a high fiber, low fat weight reduction at ideal body weight (the RD Diet). Body weight and body condition scoring is performed weekly. Body composition is determined by dual energy x-ray absorptiometry and plasma insulin and glucose are measured every 4 weeks for 12 weeks or until ideal body condition, 15-20% body fat, or initial ideal body weight is achieved.

The RD Diet is comprised of: Prescription Diet R/d
Canned 733 kcal/kg (256.55 kcal/per 350 g can) (Table 1.)

TABLE 1

| Nutrient | Dry Matter % |
|---|---|
| Protein | 25.3 |
| Fat | 8.6 |
| Carbohydrate (NFE) | 39.2 |
| Crude Fiber | 21.2 |

RD Diet Ingredients: Water, Pork By-Products, Soybean Mill Run, Rice, Pork Liver, Powdered Cellulose, Soybean Meal, Chicken Liver Flavor, Soybean Oil, Calcium Carbonate, Dicalcium Phosphate, Iron Oxide, Iodized Salt, Vitamin E Supplement, Choline Chloride, Taurine, Ascorbic Acid (source of vitamin C), L-Carnitine, Zinc Oxide, Ferrous Sulfate, Thiamine Mononitrate, Beta-Carotene, Copper Sulfate, Manganous Oxide, Niacin, Calcium Pantothenate, Vitamin B12 Supplement, Pyridoxine Hydrochloride, Biotin, Riboflavin, Vitamin D3 Supplement, Calcium Iodate, Folic Acid, Sodium Selenite.

Expected Results

Fat Oxidation

It is expected that both the leucine+a sirt activator supplemented diet and the RD diet groups will exhibit significantly greater fat oxidation than placebo, and there will be no significant difference between the leucine+a sirt activator supplemented diet and RD diet groups. It is further expected that the leucine+sirt activator group will exhibit significant loss of body fat when compared to the placebo group.

Insulin Sensitivity

It is expected that there will be a significant treatment effect on 12-week changes in insulin sensitivity, as demonstrated by significant decreases in plasma insulin and calculated homeostatic assessment model of insulin resistance (HOMA_{IR}), where HOMA_{IR} is calculated via the following formula: HOMA_{IR}=[Insulin(μU/mL)×Glucose (mM)/22.5]. The placebo group may exhibit minimal change in insulin and HOMA_{IR}, whereas the leucine+a sirt activator supplemented diet and RD diet groups, will show significant reductions in both insulin and HOMA_{IR} It is hypothesized that there will be no significant difference between the leucine+a sirt activator supplemented diet and RD diet groups.

### Example 2

### Weight Gain, Fat Oxidation, Insulin Sensitivity, and Inflammatory Stress in Animals Treated with Resveratrol and Leucine or HMB

Six week old male c57/BL6 mice were fed a high-fat diet with fat increased to 45% of energy (Research Diets D12451) for 6 weeks to induce obesity. At the end of this obesity induction period, animals were randomly divided into the following seven different diet treatment groups with 10 animals per group (overall 70 animals) and maintained on these diets for 6 weeks:

Group 1 (labeled “control group”): high-fat diet only (same as in obesity induction period (Research Diets D12451)).

This diet was modified for groups 2 to 7 in the following way:

Group 2 (labeled “low dose resveratrol”): high-fat diet mixed with 12.5 mg resveratrol/kg diet.

Group 3 (labeled “high dose resveratrol”): high-fat diet mixed with 225 mg resveratrol/kg diet.

Group 4 (labeled “low dose HMB”): high-fat diet mixed with 2 g of the calcium salt of hydroxymethylbutyrate, a naturally occurring metabolite of leucine (CaHMB).

Group 5 (labeled “low dose resveratrol plus low dose CaHMB”): high fat-diet mixed with 12.5 mg of resveratrol/kg diet and 2 g CaHMB/kg diet.

Group 6 (labeled “low dose resveratrol plus high dose HMB”): high fat-diet mixed with 12.5 mg of resveratrol/kg diet and 10 g CaHMB/kg diet.

Group 7 (labeled “low dose resveratrol plus leucine”): high fat-diet mixed with 12.5 mg of resveratrol/kg diet and leucine increased to 200% of its normal level (from 1.21 to 2.42% by weight) of the control diet

The animals were housed in polypropylene cages at a room temperature of 22±2° C. and regime of 12 h light/dark cycle. The animals had free access to water and their experimental food throughout the experiment. At the of the treatment period (6 weeks) all animals were humanely euthanized, and blood and tissues collected for further experiments.

Oxygen Consumption/Substrate Utilization:

at the end of the obesity induction period (day 0 of treatment group) and at 2 weeks and 6 weeks of treatment, oxygen consumption and substrate utilization was measured via metabolic chambers using the Comprehensive Lab Animal Monitoring Systems (CLAMS, Columbus Instruments, Columbus, Ohio) in subgroups of each treatment group. Each animal was placed in individual cages without bedding that allow automated, non-invasive data collection. Each cage is an indirect open circuit calorimeter that provides measurement of oxygen consumption, carbon dioxide production, and concurrent measurement of food intake. All mice were acclimatized to the chambers for 24 hours prior to the experiment and maintained under the regular 12:12 light:dark cycle with free access to water and food. All experiments were started in the morning and data were collected for 24 hours. Each chamber was passed with 0.61 of air/min and was sampled for 2 min at 32-minute intervals. Exhaust O₂ and CO₂ content from each chamber was compared with ambient O₂ and CO₂ content. Food consumption was measured by electronic scales.

microPET/CT (Glucose and Palmitate Uptake):

at the end of the treatment period (6 weeks of treatment) subgroups of each treatment diet group (5 animals/group, 35 animals total) were used to measure whole body glucose and palmitate uptake via PET/CT Imaging. To visualize these compounds using microPET imaging, the glucose or palmitate was labeled with fluorine-18 (108 mins half life) or carbon-11 (20 mins half life), respectively. Each mouse was fasted for 4 hours, then anesthetized using 1-3% isoflurane delivered by nose cone or in a mouse-sized induction chamber purpose-built for small animal imaging protocols. While under anesthesia the mice were injected iv with <2 mCi of each tracer, then be left for a period of time (minutes to up˜1 hour) to allow the uptake of the tracer. During the scan, mice were kept warm using a thermostatically controlled heated bed and were treated with ophthalmic ointment prior to scanning Following the live scan the mice were returned to their cage and revived. Mice were monitored constantly during this time. Following live data acquisition the mice were sacrificed by isoflurane overdose and organs harvested for further experiments.

RNA Extraction:

The Ambion ToTALLY RNA isolation kit (Ambion, Inc., Austin, Tex., USA) was used to extract total RNA from tissue according to the manufacturer's instruction. The concentration, purity and quality of the isolated RNA will be assessed by measuring the 260/280 ratio (1.8-2.0) and 260/230 ratio (close to 2.0) by using the ND-1000 Spectrophotometer (NanoDrop Technologies Inc., Del. USA). Biomarkers of the sirtuin-pathway, cytokines, and inflammatory markers (including but not limited to C-reactive protein, IL-6, MCP-1, and adiponectin molecules) can be assessed at the RNA level.

Gene Expression:

Expression of 18S, Sirt1, Sirt3, PGC1-α, cytochrome c oxidase subunit VIIc1 (COX 7), mitochondrial NADH dehydrogenase, nuclear respiratory factor 1 (NRF1), uncoupling protein (UCP2 (adipocyte)/UCP3 (myocyte), p53, AMPK, Akt/PKB, and GLUT4 is measured via quantitative real-time PCR using an ABI 7300 Real-Time PCR system (Applied Biosystems, Branchburg, N.J.) with a TaqMan® core reagent kit. All primers and probe sets can be obtained from Applied Biosystems TaqMan® Assays-on-Demand and utilized accordingly to manufacturer's instructions. Pooled RNA from each cell type are serial-diluted in the range of 0.0156-50 ng and were used to establish a standard curve; total RNA for each unknown sample is also diluted in this range. RT-PCR reactions are performed according to the instructions of the ABI Real-Time PCR system and TaqMan Real Time PCR Core Kit. Expression of each gene of interest is then normalized using the corresponding 18S quantitation.

SIRT1 Activity:

SIRT1 activity was measured by using the SIRT1 Fluorimetric Drug Discovery Kit (BML-AK555, ENZO Life Sciences International, Inc. PA, USA). In this assay, SIRT1 activity is assessed by the degree of deacetylation of a standardized substrate containing an acetylated lysine side chain. The substrate utilized is a peptide containing amino acids 379-382 of human p53 (Arg-His-Lys-Lys[Ac]), an established target of SIRT1 activity; SIRT1 activity is directly proportional to the degree of deacetylation of Lys-382. Samples were incubated with peptide substrate (25 μM), and NAD⁺ (500 μM) in a phosphate-buffered saline solution at 37° C. on a horizontal shaker for 45 minutes. The reaction was stopped with the addition of 2 mM nicotinamide and a developing solution that binds to the deacetylated lysine to form a fluorophore. Following 10 minutes incubation at 37° C., fluorescence was read in a plate-reading fluorometer at an excitation wavelength of 360 nm and an emission wavelength of 450 nm. Resveratrol (100 mM) served as a SIRT1 activator and suramin sodium (25 mM) as a SIRT1 inhibitor; wells including each were utilized as positive and negative controls in each set of reactions. A standard curve was constructed using deacetylated substrate (0-10 μM). Data was normalized to cellular protein concentration measured via BCA-assay.

Western Blot Analysis:

Tissue samples (adipose and muscle) is homogenized in ice-cold RIPA lysis buffer containing 150 mM sodium chloride, 1.0% Triton X-100, 0.5% sodium deoxycholate, 0.1% SDS and 50 mM Tris (pH 8.0), aprotinin (1 μg/ml), Leupeptin (10 μg/ml), Pepstatin A (1 μg/ml), 1 mM PMSF, 5 mM EDTA, 1 mM EGTA, 10 mM NaF, 1 mM Na Orthovanadate with an electric homogenizer, then maintained on constant agitation for 2 hours at 4° C. and centrifuged at 4,000 g for 30 min at 4° C. Aliquots of supernatants (containing 15-25 μg of total protein) is treated with 2× Laemmli sample buffer containing 100 mM dithiothreitol and run on 10% (for or 15% SDS-PAGE (for Sirt3). The resolved proteins is transferred to PVDF membrane and blocked in 5% nonfat dry milk in Tris-buffered saline containing 0.1% Tween 10, pH 7.5. After membranes are blocked, the membranes are rinsed in TBST, incubated overnight with appropriate antibody, rinsed in TBST, and incubated for 120 min with horseradish peroxidase-conjugated anti-rabbit IgG. Antibody-bound protein is visualized with enhanced chemiluminescence (ECL, Amersham).

The following antibodies are used: Anti-Sirt3 antibody (Cell Signaling Technology, Beverly, Mass.), Anti-Idh2 (Isocitrate dehydrogenase 2) (Santa Cruz, Calif.), Anti-COX antibody (Santa Cruz).

Low doses of resveratrol and HMB exerted no significant independent effect on body weight, weight gain, visceral adipose tissue mass, fat oxidation, respiratory exchange ratio (RER), or heat production, while the high dose of resveratrol significantly increased both heat production and skeletal muscle fat oxidation and decreased RER, indicating a whole-body shift towards fat oxidation (table 1); however, high dose resveratrol exerted no significant effect on body weight, weight gain, or visceral adipose tissue mass. In contrast with the lack of independent effects of a low dose of resveratrol or HMB, combining a low dose of resveratrol with either HMB or leucine resulted in significant reductions in body weight, weight gain, visceral adipose tissue mass, fat oxidation and heat production, and an associated decrease in RER (table 1).

TABLE 2
Effects of resveratrol, leucine and HMB on body weight, weight gain, adiposity
and fat oxidation in diet-induced obese mice.¹

| | | | | | Low | Low | | |
|---|---|---|---|---|---|---|---|---|
| | | | | | Resv/ | Resv/ | Low | |
| | | Low | High | Low | Low | High | Resv/ | P |
| | Control | Resveratrol² | Resveratrol³ | HMB⁴ | HMB | HMB⁵ | Leucine⁶ | value |
| Weight (g) | 40.5 ± 0.5^{a} | 40.8 ± 2.5^{a} | 38.7 ± 1.2^{a} | 40.3 ± 2.1^{a} | 36.2 ± 3.2^{b} | 34.4 ± 1.1^{b} | 38.3 ± 2.3^{b} | P < 0.05 |
| Weight gain (g) | 22.4 ± 1.1^{a} | 20.9 ± 1.5^{a} | 22.3 ± 2.4^{a} | 22.5 ± 1.2 | 18.2 ± 1.2^{b} | 19.2 ± 1.0^{b} | 19.2 ± 1.6^{b} | p < 0.01 |
| Visceral Adipose | 6556 ± 143 | 6551 ± 575^{a} | 6031 ± 323^{a} | 6184 ± 460^{a} | 5302 ± 324^{b} | 4879 ± 243^{b} | 4259 ± 321^{b} | p < 0.01 |
| Volume (mm³) | | | | | | | | |
| Fat oxidation | 1.34 ± 0.15^{a} | 1.51 ± 0.44^{a} | 2.29 ± 0.11^{b} | 1.90 ± 0.29^{a} | 2.09 ± 0.30^{b} | 1.97 ± 0.28^{b} | 1.76 ± 0.09^{a,b} | P < 0.05 |
| (PET palmitate | | | | | | | | |
| uptake; Muscle | | | | | | | | |
| SUV) | | | | | | | | |
| Respiratory | 0.850 ± 0.008^{a} | 0.847 ± 0.008^{a} | 0.825 ± 0.007^{b} | 0.844 ± 0.012^{a} | 0.815 ± 007^{b} | 0.8818 ± 0.09^{b} | 0.811 ± 0.010^{b} | P < 0.01 |
| Exchange Ratio | | | | | | | | |
| (24 hr RER) | | | | | | | | |
| Heat Production | 0.521 ± 0.015^{a} | 0.517 ± 0.014^{a} | 0.552 ± 0.015^{b} | 0.526 ± 0.011^{a} | 0.544 ± 0.010^{b} | 0.547 ± 0.009^{b} | 0.550 ± 0.012^{b} | P < 0.05 |

¹non-matching letter superscripts in each row denote significant differences at the indicated p value
²Low resveratrol: 12.5 mg resveratrol/kg diet
³High resveratrol: 225 mg resveratrol/kg diet
⁴Low HMB: 2 g hydroxymethylbutyrate (calcium salt)
⁵Leucine: Leucine increased two-fold, from 1.21% in other diets to 2.42%

Table 2 shows the effects of the dietary treatments on indices of insulin sensitivity. None of the treatments exerted any effect on plasma glucose. Neither resveratrol at either dose nor HMB exerted any significant effect on plasma insulin or on muscle glucose uptake. However, the combination of a low dose of resveratrol with either HMB or leucine resulted in significant, marked decreases in plasma insulin. This reduction in insulin with no change in plasma glucose reflects significant improvements in muscle and whole-body insulin sensitivity, as demonstrated by significant and substantial decreases in HOMA_{IR} (homeostatic assessment of insulin resistance) and corresponding increases in skeletal muscle ¹⁸F-deoxyglucose uptake (table 2 and FIG. 9).

TABLE 3
Effects of resveratrol, leucine and HMB on indices of insulin sensitivity in diet-
induced obese mice.¹

| | | | | | Low | Low | | |
|---|---|---|---|---|---|---|---|---|
| | | | | | Resv/ | Resv/ | Low | |
| | | Low | High | Low | Low | High | Resv/ | |
| | Control | Resveratrol² | Resveratrol³ | HMB⁴ | HMB | HMB⁵ | Leucine⁶ | P value |
| Glucose (mM) | 4.97 ± 0.60^{a} | 5.14 ± 0.85^{a} | 5.14 ± 0.75^{a} | 4.28 ± 0.49^{a} | 4.67 ± 0.49^{a} | 4.33 ± 0.41^{a} | 5.05 ± 0.92^{a} | NS |
| Insulin (μU/mL) | 12.5 ± 3.4^{a} | 10.4 ± 1.6^{a} | 10.1 ± 2.7^{a} | 8.3 ± 1.1^{a} | 5.8 ± 0.7^{b} | 3.9 ± 1.2^{b} | 5.5 ± 1.4^{b} | P < 0.005 |
| HOMA_{IR} | 2.61 ± 0.82^{a} | 2.41 ± 0.66^{a} | 0.59 ± 0.26^{b} | 1.93 ± 0.32^{a} | 1.18 ± 0.25^{c} | 0.87 ± 0.31^{b} | 1.14 ± 0.37^{c} | P < 0.01 |
| Muscle Glucose | 3.64 ± 0.88^{a} | 3.63 ± 1.29^{a} | 3.87 ± 0.32^{a} | 2.99 ± 0.42^{a} | 5.90 ± 0.41^{b} | 5.93 ± 1.63^{b} | 5.68 ± 0.75^{b} | P < 0.02 |
| Uptake (¹⁸F- | | | | | | | | |
| deoxyglucose SUV) | | | | | | | | |

¹non-matching letter superscripts in each row denote significant differences at the indicated p value
²Low resveratrol: 12.5 mg resveratrol/kg diet
³High resveratrol: 225 mg resveratrol/kg diet
⁴Low HMB: 2 g hydroxymethylbutyrate (calcium salt)
⁵Leucine: Leucine increased two-fold, from 1.21% in other diets to 2.42%

FIG. 10 shows the effects of dietary treatments on adipose tissue Sirt1 activity. Neither resveratrol nor HMB exerted significant independent effects on Sirt1 activity, although high dose resveratrol exhibited a non-significant trend towards an increase. In contrast, combining a low dose of resveratrol with either HMB or leucine resulted in ˜two-fold increases in tissue Sirt1 activity. Such sirtuin activation would be anticipated to reduce inflammatory response. Consistent with this concept, the high dose of resveratrol significantly reduced circulating IL-6, while the combination of a low dose of resveratrol (which exerted no independent effect) with HMB resulted in a markedly greater lowering of IL-6 (table 3). Similarly, while neither HMB nor a low dose of resveratrol exerted any effect on MCP-1 or c-reactive protein, the combination of a low dose of resveratrol with either HMB or leucine resulted in significant decreases in both inflammatory biomarkers.

Moreover, the anti-inflammatory cytokine adiponectin was increased in response to a low dose of resveratrol in combination with either HMB or leucine, while the individual components at these doses exerted no significant effect (table 3).

TABLE 4
Effects of resveratrol, leucine and HMB on inflammatory biomarkers in diet-
induced obese mice.¹

| | | | | | Low | Low | | |
|---|---|---|---|---|---|---|---|---|
| | | | | | Resv/ | Resv/ | Low | |
| | | Low | High | Low | Low | High | Resv/ | |
| | Control | Resveratrol² | Resveratrol³ | HMB⁴ | HMB | HMB⁵ | Leucine⁶ | P value |
| C-reactive | 95.6 ± 9.6^{a} | 134.8 ± 8.5^{a} | 123.9 ± 35.3^{a} | 98.6 ± 5.1 | 67.4 ± 12.2^{b} | 58.3 ± 12.4^{b} | 55.9 ± 17.7^{b} | P < 0.01 |
| protein (ng/mL) | | | | | | | | |
| IL-6 (pg/mL) | 29.0 ± 6.4^{a} | 23.2 ± 2.9^{a} | 14.1 ± 1.3^{b} | 19.9 ± 3.1^{a} | 6.9 ± 1.2^{c} | 4.5 ± 2.6^{c} | 11.2 ± 4.1^{b} | P < 0.005 |
| MCP-1 (pg/mL) | 115.8 ± 19.7^{a} | 104.4 ± 16.5^{a} | 27.3 ± 6.8^{b} | 116.8 ± 9.3^{a} | 24.2 ± 6.2^{b} | 15.2 ± 3.7^{b} | 34.9 ± 5.9^{b} | P < 0.001 |
| Adiponectin | 11.0 ± 0.9^{a} | 12.4 ± 1.1 | 14.8 ± 1.8^{b} | 11.1 ± 1.6^{a} | 14.1 ± 0.8^{b} | 16.3 ± 3.0^{b} | 14.5 ± 1.0^{b} | P < 0.03 |
| (ng/mL) | | | | | | | | |

¹non-matching letter superscripts in each row denote significant differences at the indicated p value
²Low resveratrol: 12.5 mg resveratrol/kg diet
³High resveratrol: 225 mg resveratrol/kg diet
⁴Low HMB: 2 g hydroxymethylbutyrate (calcium salt)
⁵Leucine: Leucine increased two-fold, from 1.21% in other diets to 2.42%

Collectively, these data demonstrate synergy between low doses of resveratrol and leucine or its metabolite HMB in activating Sirt1 and Sirt1-dependent outcomes. These include increased fat oxidation and attenuation of adiposity and obesity, augmentation of insulin sensitivity and reversal of insulin resistance, and attenuation of systemic inflammatory stress.

### REFERENCES

1. Bartges J W, Kirk C A, Lauten S. Calculating a patient's nutritional requirements. Vet Med 2004; 99:632.
2. Laflamme D. Development and validation of a body condition score system for dogs. Canine Practice 1997; 22:10-15.
3. Mawby D I, Bartges J W, d'Avignon A, et al. Comparison of various methods for estimating body fat in dogs. J Am Anim Hosp Assoc 2004; 40:109-114.
4. Ionut V, Liu H, Mooradian V, et al. Novel canine models of obese prediabetes and mild type 2 diabetes. Am J Physiol Endocrinol Metab 2010; 298:E38-48.

It should be understood from the foregoing that, while particular implementations have been illustrated and described, various modifications can be made thereto and are contemplated herein. It is also not intended that the invention be limited by the specific examples provided within the specification. While the invention has been described with reference to the aforementioned specification, the descriptions and illustrations of the preferable embodiments herein are not meant to be construed in a limiting sense. Furthermore, it shall be understood that all aspects of the invention are not limited to the specific depictions, configurations or relative proportions set forth herein which depend upon a variety of conditions and variables. Various modifications in form and detail of the embodiments of the invention will be apparent to a person skilled in the art. It is therefore contemplated that the invention shall also cover any such modifications, variations and equivalents.

What is claimed is:

1. A pet food composition comprising:
a. about 0.05 to 5 wt % of leucine and/or about 0.005 to 1 wt % of one or more leucine metabolites; and
b. about 0.0005 to 0.05 wt % of a sirtuin activator.

2. A pet food, treat or supplement composition comprising:
a. at least about 50 mg of leucine and/or at least about 5 mg of one or more leucine metabolites; and
b. at least about 1 mg of a sirtuin activator.

3. A pet food composition comprising:
a. about 0.05 to 5 wt % of leucine and/or about 0.005 to 1 wt % of one or more leucine metabolites;
b. about 0.0005 to 0.05 wt % of a sirtuin activator; and
c. an additional component selected from the group consisting of omega-3 fatty acid, eicosapentanoic acid, choline, manganese, methionine, cysteine, L-carnitine, lysine, alpha lipoic acid, dimethylaminoethanol, pyruvic acid, actyl L-carnitine, L-carnitine, conjugated linoleic acid, diacylglyceride, chondroitin, glucosamine, ginger (or extract thereof), chicory pulp, and myrtle.

4. A pet food, treat or supplement composition comprising:
a. at least about 50 mg of leucine and/or at least about 5 mg of one or more leucine metabolites;
b. at least about 1 mg of a sirtuin activator; and
c. an additional component selected from the group consisting of omega-3 fatty acid, eicosapentanoic acid, choline, manganese, methionine, cysteine, L-carnitine, lysine, alpha lipoic acid, dimethylaminoethanol, pyruvic acid, actyl L-carnitine, L-carnitine, conjugated linoleic acid, diacylglyceride, chondroitin, glucosamine, ginger (or extract thereof), chicory pump, and myrtle.

5. The pet food composition of claim 3, wherein said omega-3 fatty acid comprises at least about 0.05%, at least about 3.5%, or between about 0.05 to 3.5% of the weight of the pet food composition.

6. The pet food composition of claim 3, wherein said eicosapentanoic acid comprises at least about 0.4 wt % of the pet food composition.

7. The pet food composition of claim 3, wherein said vitamin E comprises at least about 100 ppm of the pet food composition.

8. The pet food composition of claim 3, wherein said vitamin C comprises at least about 50 ppm of the pet food composition.

9. The pet food composition of claim 3, wherein said L-carnitine comprises at least about 50 ppm of the pet food composition.

10. The pet food composition of claim 3, wherein said alpha-lipoic acid comprises at least about 25 ppm of the pet food composition.

11. The pet food composition of claim 3, wherein said choline comprises at least about 1000 ppm of the pet food composition.

12. The pet food composition of claim 3, wherein said manganese comprises at least about 50 ppm, or from about 50 ppm to about 150 ppm, or from about 100 ppm to about 150 ppm, or from about 100 ppm to about 110 ppm of the pet food composition.

13. The pet food composition of claim 3, wherein said methionine comprises at least about at least about 0.4 to 1.5% methionine by weight of the pet food composition.

14. The pet food composition of claim 3, wherein said lysine comprises at least about 0.4%, between about 0.4 to 2%, between about 0.9 to 2%, or between about 0.9 to 1.2% by weight of the pet food composition.

15. The pet food composition of claim 3, wherein said amount of ginger or extract thereof is about 0.005 to 12% by weight of the pet food composition.

16. The pet food composition of claim 3, wherein said chicory pulp comprises from 0.5 to 20% dry weight of the pet food composition.

17. The pet food composition of claim 3, wherein said chondroitin comprises at least about 0.5 wt % of the pet food composition.

18. The pet food composition of claim 3, wherein said glucosamine comprises at least about 0.3 wt % of the pet food composition.

19. The pet food composition of claim 3, wherein said myrtle comprises at least about 1% or between about 1 to 10% by weight of the pet food composition.

20. The pet food composition of claim 4, wherein said omega-3 fatty acid is present in an amount of at least about 250 mg.

21. The pet food composition of claim 4, wherein said eicosapentanoic acid is present in an amount of at least about 300 mg.

22. The pet food composition of claim 4, wherein said vitamin E is present in an amount of at least about 200 mg

23. The pet food composition of claim 4, wherein said vitamin C is present in an amount of at least about 250 mg.

24. The pet food composition of claim 4, wherein said L-carnitine is present in an amount of at least about 250 mg.

25. The pet food composition of claim 4, wherein said alpha-lipoic acid is present in an amount of at least about 180 mg.

26. The pet food composition of claim 4, wherein said choline is present in an amount of at least about 1000 mg.

27. The pet food composition of claim 4, wherein said manganese is present in an amount of at least about 50 mg, or from about 50 mg to about 150 mg, or from about 100 mg to about 150 mg, or from about 100 mg to about 110 mg.

28. The pet food composition of claim 4, wherein said methionine is present in an amount of at least about 7.5 mg.

29. The pet food composition of claim 4, wherein said lysine is present in an amount of at least about 400 mg.

30. The pet food composition of claim 4, wherein said amount of ginger or extract thereof is present at an amount of at least about 500 mg.

31. The pet food composition of claim 4, wherein said chondroitin is present in an amount of at least about 500 mg.

32. The pet food composition of claim 4, wherein said glucosamine is present in an amount of at least about 300 mg.

33. The pet food composition of claim 4, wherein said myrtle is present in an amount of at least about 750 mg.

34. A pet food composition comprising:
a. about 0.05 to 5 wt. % of leucine and/or about 0.005 to 1 wt % of one or more leucine metabolites on a dry matter basis;
b. about 0.0005 to 0.05 wt. % of a sirtuin activator on a dry matter basis;
c. about 26 to 35 wt. % of crude protein on a dry matter basis;
d. about 7.5 to 8.5 wt. % of crude fat on a dry matter basis;
e. about 20 to 30 wt. % of total dietary fiber on a dry matter basis; and
f. about 10 to 20 wt. % of crude fiber on a dry matter basis.

35. A pet food composition comprising:
N pieces wherein N pieces provide the complete and balanced daily nutritional requirements of an animal, and N equals 1 to less than 15 pieces,
wherein each piece has a caloric content between 50 to 2500 kcal, and
wherein N pieces comprise (a) at least about 100 mg of leucine and/or at least about 10 mg of one or more leucine metabolites and (b) at least about 1 mg of a sirtuin activator.

36. A diet for promoting comprehensive weight management in companion animals comprising:
a first stage pet food composition for promoting weight loss and a second stage pet food composition for maintaining the weight loss,
(a) said first stage pet food composition comprising, on a dry matter basis,
about 35 to 70% by weight of a protein,
about 4 to 10% by weight of a fat,
about 2 to 25% by weight of a fiber,
about 10 to 35% by weight of a carbohydrate,
about 0.05 to 5% by weight of leucine and/or about 0.005 to 1% by weight of one or more leucine metabolites,
about 0.0005 to 0.05% by weight of a sirtuin activator, and
about 0.1 to 2% by weight of a functional ingredient,
wherein said functional ingredient is selected from the group consisting of L-carnitine and conjugated linoleic acid; and
(b) said second stage pet food composition comprising, on a dry matter basis,
about 20 to 35% by weight of a protein,
about 4 to 10% by weight of a fat,
about 2 to 25% by weight of a fiber,
about 25 to 70% by weight of a carbohydrate, and
about 0.1 to 2% by weight of a functional ingredient,
wherein said functional ingredient is selected from the group consisting of L-carnitine and conjugated linoleic acid,
wherein the protein content of the second stage pet food composition is about 10 to 45% less than the protein content of the first stage pet food composition.

37. A pet food composition comprising:
a. a live probiotic microorganism;
b. about 0.05 to 5 wt % of leucine and/or about 0.005 to 1 wt % of one or more leucine metabolites; and
c. about 0.0005 to 0.05 wt % of A sirtuin activator.

38. A pet food composition comprising:
a. a live probiotic microorganism;
b. at least about 50 mg of leucine and/or at least about 5 mg of one or more leucine metabolites by weight of the pet food composition; and
c. at least about 1 mg of a sirtuin activator by weight of the pet food composition.

39. The pet food composition of claim 37 or 38, wherein the probiotic microorganism is selected from the group consisting of Bifidobacterium, Bacteroides, Clostridium, Fusobacterium, Melissococcus, Propionibacterium, Streptococcus, Enterococcus, Lactococcus, Staphylococcus, Peptostrepococcus, Bacillus, Pediococcus, Micrococcus, Leuconostoc, Weissella, Aerococcus, Oenococcus, or Lactobaccillus.

40. The pet food composition of claim 37 or 38, wherein said composition comprises a starch source

41. The pet food composition of claim 37 or 38, wherein said starch source has a degree of gelatinization less than about 7.5 Joules/g of starch.

42. A pet food compositions comprising:
a. about 0.05 to 5 wt % of leucine and/or about 0.005 to 1 wt % of one or more leucine metabolites;
b. about 0.0005 to 0.05 wt % of a sirtuin activator; and
c. a pre-selected balance of metabolizable cations to metabolizable anions, wherein the DCAB is between about 50 to 300.

43. A method for treating a puppy susceptible to or suffering from diarrhea and/or loose stool comprising:
a. feeding the puppy a food composition comprising (i) about 0.05 to 5 wt % of leucine and/or about 0.005 to 1 wt % of one or more leucine metabolites and (ii) about 0.0005 to 0.05 wt % of a sirtuin activator; and
b. adjusting the balance of metabolizable cations to metabolizable anions consumed by the puppy by an amount sufficient to improve stool quality by increasing the balance of metabolizable cations to metabolizable anions consumed by the puppy to produce firmer stool.

44. A pet food composition for reducing odor of stool of a companion animal comprising:
a. stool odor-reducing effective amount of ginger or an extract thereof;
b. about 0.05 to 5 wt % of leucine and/or about 0.005 to 1 wt % of one or more leucine metabolites; and
c. about 0.0005 to 0.05 wt % of a sirtuin activator.

45. The pet food composition of claim 44, wherein said amount of ginger or extract thereof is present at an amount of at least about 500 mg.

46. The pet food composition of claim 44, wherein said amount of ginger or extract thereof is about 0.005 to 12% by weight.

47. A method for reducing odor of stool of a companion animal, the method comprising causing the animal to ingest a pet food composition comprising:
a. a stool odor-reducing effective amount of ginger or an extract thereof;
b. about 0.05 to 5 wt % of leucine and/or about 0.005 to 1 wt % of one or more leucine metabolites; and
c. about 0.0005 to 0.05 wt % of a sirtuin activator.

48. The method of claim 47, wherein said amount of ginger or extract thereof is about 0.005 to 12% by weight.

49. The method of claim 47, wherein said amount of ginger or extract thereof is present at an amount of at least about 500 mg.

50. A pet food composition which comprises:
a. chicory pulp in an amount which: i) maintains good feces quality or improves the feces quality of a pet and/or ii) maintains good gastrointestinal tract health and/or improves the gastrointestinal tract health of a pet;
b. about 0.05 to 5 wt % of leucine and/or about 0.005 to 1 wt % of one or more leucine metabolites; and
c. about 0.0005 to 0.05 wt % of a sirtuin activator.

51. The pet food composition of claim 50, wherein said chicory pulp comprises from 0.5 to 20% dry weight of the pet food product

52. The pet food composition of claim 50, wherein said chicory pulp comprises from 2 to 10% dry weight of the pet food product.

53. The pet food composition of claim 50, wherein said chicory pulp is present at an amount of at least about 350 mg.

54. A pet food composition comprising
a. an inner layer comprising kibble;
b. an outer layer;
c. about 0.05 to 5% of leucine and/or about 0.005 to 1% of one or more leucine metabolites by weight of the pet food composition; and
d. about 0.0005 to 0.05% of a sirtuin activator by weight of the pet food composition.

55. The pet food composition of claim 54, wherein the inner layer comprises
a. about 0.05 to 5% of leucine and/or about 0.005 to 1% of one or more leucine metabolites by weight of the pet food composition; and
b. about 0.0005 to 0.05% of a sirtuin activator by weight of the pet food composition.

56. The pet food composition of claim 54, wherein the outer layer comprises
a. about 0.05 to 5% of leucine and/or about 0.005 to 1% of one or more leucine metabolites by weight of the pet food composition; and
b. about 0.0005 to 0.05% of a sirtuin activator by weight of the pet food composition.

57. A pet food or supplement composition comprising
a. an inner layer comprising kibble;
b. an outer layer; and
c. at least about 50 mg of leucine and/or at least about 5 mg of one or more leucine metabolites by weight of the pet food composition; and
d. at least about 1 mg of a sirtuin activator by weight of the pet food composition.

58. The pet food or supplement composition of claim 57, wherein the inner layer comprises
a. at least about 50 mg of leucine and/or at least about 5 mg of one or more leucine metabolites by weight of the pet food composition; and
b. at least about 1 mg of a sirtuin activator by weight of the pet food composition.

59. The pet food or supplement composition of claim 57, wherein the outer layer comprises
a. at least about 50 mg of leucine and/or at least about 5 mg of one or more leucine metabolites by weight of the pet food composition; and
b. at least about 1 mg of a sirtuin activator by weight of the pet food composition.

60. A pet food composition comprising:
a. about 0.05 to 5 wt % of leucine and/or about 0.005 to 1 wt % of one or more leucine metabolites;
b. about 0.0005 to 0.05 wt % of a sirtuin activator; and
c. a palatability enhancer selected from the group consisting of butyric acid, 3-methylbutyric acid, tetrasodium pyrophosphate, 2-piperidione, 2,3 pentanedione, 2-ethyl-3,5-dimethylpyrazine, furfural, sulfurol, and indole.

61. A pet food or supplement composition comprising
a. at least about 50 mg of leucine and/or at least about 5 mg of one or more leucine metabolites by weight of the pet food composition;
b. at least about 1 mg of a sirtuin activator by weight of the pet food composition; and
c. a palatability enhancer selected from the group consisting of butyric acid, 3-methylbutyric acid, tetrasodium pyrophosphate, 2-piperidione, 2,3 pentanedione, 2-ethyl-3,5-dimethylpyrazine, furfural, sulfurol, and indole.

62. A pet food composition comprising:
an outer layer joined to an inner layer, wherein the outer layer is harder than the inner layer;
about 0.05 to 5 wt % of leucine and/or about 0.005 to 1 wt % of one or more leucine metabolites; and
about 0.0005 to 0.05 wt % of a sirtuin activator.

63. A pet food composition comprising:
a structurant for providing a textured appearance and feel;
about 0.05 to 5 wt % of leucine and/or about 0.005 to 1 wt % of one or more leucine metabolites; and
about 0.0005 to 0.05 wt % of a sirtuin activator.

64. A pet food composition comprising:
a textured layer bonded to a base layer, wherein the textured layer comprises textured components bonded to the base layer;
about 0.05 to 5 wt % of leucine and/or about 0.005 to 1 wt % of one or more leucine metabolites; and
about 0.0005 to 0.05 wt % of a sirtuin activator.

65. A pet food composition comprising:
less than about 19% on a dry weight basis of carbohydrate;
about 0.05 to 5 wt % of leucine and/or about 0.005 to 1 wt % of one or more leucine metabolites; and
about 0.0005 to 0.05 wt % of a sirtuin activator.

66. The pet food composition of claim 65, wherein the pet food composition is dimensionally stable.

67. A method for feeding a pet comprising:
a. providing, over an extended and preselected period of time, different food compositions to said animal in which each composition provides an enriched source of fat, protein or carbohydrate, such that said animal can select and consume different and preferred quantities of each said food compositions in order to achieve an preferred consumption of fat, protein and carbohydrate for said animal;
wherein at least one of said food compositions comprises about 0.05 to 5 wt % of leucine and/or about 0.005 to 1 wt % of one or more leucine metabolites; and about 0.0005 to 0.05 wt % of a sirtuin activator;
b. allowing said animal to consume the different and preferred quantities of fat, protein and carbohydrate from each of said compositions over the extended preselected period of time; and
c. determining, from the consumed amount of fat, protein and carbohydrate from each of said compositions, a customized dietary regime that provides the preferred macronutrient content of a diet for said individual animal.

68. A computer-readable medium comprising code that, upon execution by one or more processors, implements a method of producing a customized dry pet food composition formulated from a dry pet food kibble recipe and selected functional ingredients, the formulation being selected on the basis of an individual pet's attributes and physical conditions, the method comprising:
a. receiving information on the individual pet's attributes and physical conditions;
b. selecting a predetermined volume of dry kibble pieces from a plurality different formulations of pre-made dry kibble pieces;
c. selecting one or more functional ingredients from a plurality functional ingredients;
d. coating the predetermined volume of dry kibble pieces with the one or more functional ingredients; and
e. packaging and labeling the predetermined volume of coated dry kibble pieces;
wherein the selection of the predetermined volume of dry kibble pieces and one or more functional ingredients is based on the individual pet's attributes and physical conditions to provide the customized dry pet food product, and
wherein the coating and/or the kibble pieces comprise leucine and/or a leucine metabolite and a sirtuin activator.

69. The method of claim 68, wherein
a. the leucine, if present, is in an amount that is about 0.05 to 5 wt %;
b. the leucine metabolites, if present, are in an amount that is about 0.005 to 1 wt %; and
c. the a sirtuin activator, if present, is in an amount that is about 0.0005 to 0.05 wt %.

70. A method of producing a customized dry pet food product formulated from a dry pet food kibble recipe and selected functional ingredients, the formulation being selected on the basis of an individual pet's attributes and physical conditions, the method comprising:
a. providing a plurality of different formulations of pre-made dry kibble pieces;
b. selecting a predetermined volume of dry kibble pieces from the plurality of different formulations of pre-made dry kibble pieces;
c. providing a plurality of functional ingredients;
d. coating the volume of dry kibble pieces with one or more of the plurality of functional ingredients; and
e. packaging and labeling the predetermined volume of coated dry kibble pieces;
wherein the selection of the predetermined volume of dry kibble pieces and the one or more functional ingredients is based on the individual pet's attributes and physical conditions to provide the customized dry pet food product, and
wherein the coating and/or the kibble pieces comprise leucine and/or a leucine metabolite and a sirtuin activator.

71. The method of claim 70, wherein
a. the leucine, if present, is in an amount that is about 0.05 to 5 wt %;
b. the leucine metabolites, if present, are in an amount that is about 0.005 to 1 wt %; and
c. the a sirtuin activator, if present, is in an amount that is about 0.0005 to 0.05 wt %.

72. A diet for companion animals comprising:
a first stage pet food composition and a second stage pet food composition for maintaining the weight loss, wherein each of said first stage pet food composition and said second stage pet food composition comprise, on a dry matter basis, about 0.05 to 5 wt % of leucine and/or about 0.005 to 1 wt % of one or more leucine metabolites, and about 0.0005 to 0.05 wt % of a sirtuin activator, and
wherein said second stage pet food comprises at least about 5% higher fat content compared to said first stage pet food.

73. A vegetarian pet food composition comprising:
a. a vegetarian kibble which incorporates a non-meat based flavor-enhancing additive;
b. about 0.05 to 5 wt % of leucine and/or about 0.005 to 1 wt % of one or more leucine metabolites; and
c. about 0.0005 to 0.05 wt % of a sirtuin activator.

74. A vegetarian pet food or supplement composition comprising:
a. a vegetarian kibble which incorporates a non-meat based flavor-enhancing additive;
b. at least about 50 mg of leucine and/or at least about 5 mg of one or more leucine metabolites by weight of the pet food composition; and
c. at least about 1 mg of a sirtuin activator by weight of the pet food composition.

75. A multi-component pet food composition comprising two or more compartmentalized food compositions, wherein the at least two compartmentalized compositions differ in their content in at least two of fat, protein or carbohydrate, and further wherein one of the two or more compartmentalized compositions comprises about 0.05 to 5 wt % of leucine and/or about 0.005 to 1 wt % of one or more leucine metabolites, and about 0.0005 to 0.05 wt % of a sirtuin activator.

76. A pet food composition for increasing the shelf life of a physically discrete dry pet food comprising:
a. a coat covering the physically discrete pet food composition comprising a polymer film, wherein the film or an agent in the film protects the composition from oxidation decomposition and/or protects the composition from bacterial growth, wherein the film comprises a starch/synthetic polymer selected from the group consisting of starch/polyethylene, and starch/low-density polyethylene, and wherein the thickness of said coat is 1-2000 microns;
b. about 0.05 to 5 wt % of leucine and/or about 0.005 to 1 wt % of one or more leucine metabolites; and
c. about 0.0005 to 0.05 wt % of a sirtuin activator.

77. A method for increasing the shelf life of a physically discrete dry pet food composition comprising:
coating the physically discrete pet food composition with a polymer film, wherein the film or an agent in the film protects the composition from oxidation decomposition and/or protects the composition from bacterial growth,
wherein the film comprises a starch/synthetic polymer selected from the group consisting of starch/polyethylene, and starch/low-density polyethylene, wherein the thickness of said coating is 1-2000 microns; and
wherein the pet food composition comprises about 0.05 to 5 wt % of leucine and/or about 0.005 to 1 wt % of one or more leucine metabolites and about 0.0005 to 0.05 wt % of a sirtuin activator.

78. A method for making kibble at one location and finishing the pet food at another location comprising:
a. forming a dry, stable intermediate of the pet food composition at a first location;
b. finishing the dry, stable intermediate pet food composition to form a finished pet food composition at a second location remote from the first location,
wherein said finished pet food composition contains about 0.05 to 5 wt % of leucine and/or about 0.005 to 1 wt % of one or more leucine metabolites and about 0.0005 to 0.05 wt % of a sirtuin activator.

79. A hypoallergenic pet food composition comprising:
a. proteinaceous component that has been hydrolyzed whereby said component is rendered hypoallergenic to a pet, wherein said proteinaceous component is made up of polypeptides and free amino acids having an average molecular weight of less than about 3,000 Daltons;
b. about 0.05 to 5 wt % of leucine and/or about 0.005 to 1 wt % of one or more leucine metabolites; and
c. about 0.0005 to 0.05 wt % of a sirtuin activator.

80. A method of administering a pet food, treat, supplement, or drink to a pet comprising administering a composition of any of the preceding claims to the pet.

81. A method of facilitating weight loss in a pet in need thereof comprising administering a composition of any of the preceding claims to the pet, wherein the pet experiences weight loss.

82. The method of claim 81, wherein the pet loses about 10% of its weight relative to the weight of the pet prior to administration of the composition.

83. A method of facilitating fat loss in a pet in need thereof comprising administering a composition of any of the preceding claims to the pet, wherein the pet experiences fat loss.

84. The method of claim 82, wherein the pet loses about 10% fat relative to the amount of fat prior to administration of the composition.

85. A method of reducing body condition score of a pet in need thereof comprising administering a composition of any of the preceding claims to the pet, wherein the pet experiences a reduction in body condition score.

86. The method of claim 85, wherein the pet's body condition score is reduced by about 2 relative to the pet's body condition score prior to administration of the composition.

87. A method of increasing production of irisin in a pet comprising administering to the pet a pet food, pet treat, pet snack or pet drink composition that comprises:
a. about 0.05 to 5 wt % of leucine and/or about 0.005 to 1 wt % of one or more leucine metabolites; and
b. about 0.0005 to 0.05 wt % of a sirtuin activator, wherein the production of irisin in the pet is increased.

88. A method of increasing production of irisin in a pet comprising administering to the pet a pet food, pet treat, pet snack or pet drink composition that comprises:
a. at least about 50 mg of leucine and/or at least about 5 mg of one or more leucine metabolites;
b. about 1 mg of a sirtuin activator, wherein the production of irisin in the pet is increased.

89. A method of increasing insulin sensitivity in a pet comprising administering to the pet a pet food, pet treat, pet snack or pet drink composition that comprises:
a. about 0.05 to 5 wt % of leucine and/or about 0.005 to 1 wt % of one or more leucine metabolites; and
b. about 0.0005 to 0.05 wt % of a sirtuin activator,
wherein the insulin sensitivity of the pet is increased.

90. A method of increasing insulin sensitivity in a pet comprising administering to the pet a pet food, pet treat, pet snack or pet drink composition that comprises:
a. at least about 50 mg of leucine and/or at least about 5 mg of one or more leucine metabolites;
b. at least about 1 mg of a sirtuin activator,
wherein the insulin sensitivity in the pet is increased.

91. A method of reducing inflammation in a pet comprising administering to the pet a pet food, pet treat, pet snack or pet drink composition that comprises:
a. about 0.05 to 5 wt % of leucine and/or about 0.005 to 1 wt % of one or more leucine metabolites; and
b. about 0.0005 to 0.05 wt % of a sirtuin activator, wherein the inflammation in the pet is reduced.

92. A method reducing inflammation in a pet comprising administering to the pet a pet food, pet treat, pet snack or pet drink composition that comprises:
a. at least about 50 mg of leucine and/or at least about 5 mg of one or more leucine metabolites; and
b. about 1 mg of a sirtuin activator,
wherein the inflammation in the pet is reduced.

93. A method of reducing and/or preventing diabetes in a pet comprising administering a pet food, pet treat, pet snack or pet drink composition that comprises:
a. about 0.05 to 5 wt % of leucine and/or about 0.005 to 1 wt % of one or more leucine metabolites; and
b. about 0.0005 to 0.05 wt % of a sirtuin activator,
wherein the effects of diabetes in the pet are reduced or prevented.

94. A method of reducing and/or preventing diabetes in a pet comprising administering a pet food, pet treat, pet snack or pet drink composition that comprises:
a. at least about 50 mg of leucine and/or at least about 5 mg of one or more leucine metabolites; and
b. at least about 1 mg of a sirtuin activator,
wherein the effects of diabetes in the pet are reduced or prevented.

95. A pet food composition comprising:
a. about 0.05 to 5 wt % of leucine and/or about 0.005 to 1 wt % of one or more leucine metabolites; and
b. about 0.0005 to 0.05 wt % of a sirtuin activator; and
c. about 1.5 g/kg metformin, about 0.75 g/kg metformin, about 0.25 g/kg metformin.

96. A pet food, treat or supplement composition comprising:
a. at least about 50 mg of leucine and/or at least about 5 mg of one or more leucine metabolites;
b. at least about 1 mg of a sirtuin activator; and
c. at least about 125 mg metformin

97. The pet food, treat, or supplement composition or method of any of the preceding claims, wherein the leucine metabolite is selected from the group consisting of keto-isocaproic acid (KIC), alpha-hydroxy-isocaproic acid, and hydroxymethylbutyrate (HMB).

98. The pet food, treat, or supplement composition or method of any of the preceding claims, wherein the sirtuin pathway activator is a hydroxycinnamic acid or a stilbene.

99. The pet food, treat, or supplement composition or method of any of the preceding claims, wherein the sirtuin pathway activator is resveratrol.

100. The pet food, treat, or supplement composition or method of any of the preceding claims, wherein the composition is substantially free of alanine in free form or salt form.

101. The pet food, treat, or supplement composition or method of any of the preceding claims, wherein the composition is substantially free of glutamic acid in free form or salt form.

102. The pet food, treat, or supplement composition or method of any of the preceding claims, wherein the composition is substantially free of glycine in free form or salt form.

103. The pet food, treat, or supplement composition or method of any of the preceding claims, wherein the composition is substantially free of proline in free form or salt form.

104. The pet food, treat, or supplement composition or method of any of the preceding claims, wherein the composition is substantially free of alanine in free form or salt form.

105. The pet food, treat, or supplement composition or method of any of the preceding claims, wherein the composition is substantially free of non-leucine amino acids in free of salt form.

106. The pet food, treat, or supplement composition or method of any of the preceding claims, wherein the composition comprises less than 1% of alanine in free form or salt form.

107. The pet food, treat, or supplement composition or method of any of the preceding claims, wherein the composition comprises less than 1% of glutamic acid in free form or salt form.

108. The pet food, treat, or supplement composition or method of any of the preceding claims, wherein the composition comprises less than 1% of glycine in free form or salt form.

109. The pet food, treat, or supplement composition or method of any of the preceding claims, wherein the composition comprises less than 1% of proline in free form or salt form.

110. The pet food, treat, or supplement composition or method of any of the preceding claims, wherein the composition comprises less than 1% of non-leucine amino acids in free form or salt form.

111. The pet food, treat, or supplement composition or method of any of the preceding claims, wherein the amount of leucine is between about 50-400, 50-300, 50-250, or 50-200 mg.

112. The pet food, treat, or supplement composition or method of any of the preceding claims, wherein the amount of leucine metabolites is between about 5-50, 5-25, or 5-10 mg.

113. The pet food, treat, or supplement composition or method of any of the preceding claims, wherein the amount of sirtuin activator is between about 1-10, 1-5, or 1-2 mg.
